(19) United States
(12) Patent Application Publication
(10) Pub. No.: US 2025/0161413 A1
(43) Pub. Date: May 22, 2025

(54) METHODS FOR DECREASING PATHOLOGIC ALPHA-SYNUCLEIN USING AGENTS THAT MODULATE FNDC5 OR BIOLOGICALLY ACTIVE FRAGMENTS THEREOF

(71) Applicants: Dana-Farber Cancer Institute, Inc., Boston, MA (US); The Johns Hopkins University, Baltimore, MD (US)

(72) Inventors: Bruce M. Spiegelman, Waban, MA (US); Ted M. Dawson, Baltimore, MD (US)

(21) Appl. No.: 18/838,814

(22) PCT Filed: Feb. 16, 2023
(86) PCT No.: PCT/US23/13202

§ 371 Date: Aug. 15, 2024

Related U.S. Application Data
(60) Provisional application No. 63/310,873, filed on Feb. 16, 2022.

(51) Int. Cl. A61K 38/22; A61K 9/00; A61K 48/00; A61P 25/16; C12N 15/86

(52) U.S. Cl. CPC A61K 38/22; A61K 9/0019; A61K 48/005; A61K 48/0075; A61K 48/0083; A61P 25/16; C12N 15/86; C12N 2750/14143

(57) ABSTRACT

The present invention provides methods for preventing or reducing degeneration of dopaminergic neurons and/or preventing or ameliorating at least one motor deficit in a subject in need thereof, such as in a subject with α-synucleinopathy, using agents that modulate Fndc5 or biologically active fragments thereof, such as irisin.

### CROSS-REFERENCE TO RELATED APPLICATIONS

This application claims the benefit of priority to U.S. Provisional Application Ser. No. 63/310,873, filed on Feb. 16, 2022; the entire contents of said application is incorporated herein in its entirety by this reference.

### BACKGROUND OF THE INVENTION

Parkinson's Disease (PD) is a chronic neurodegenerative disorder characterized by progressive worsening of motor symptoms including bradykinesia, resting tremor and rigidity (A. Berardelli, J. C. Rothwell, P. D. Thompson, M. Hallett, Pathophysiology of bradykinesia in Parkinson's disease. Brain 124, 2131-2146 (2001); J. Jankovic, Parkinson's disease: clinical features and diagnosis. J Neurol Neurosurg Psychiatry 79, 368-376 (2008). Non-motor symptoms often predate and accompany the motor symptoms and include autonomic dysfunction and neuropsychiatric sequalae (M. Asahina, E. Vichayanrat, D. A. Low, V. Iodice, C. J. Mathias, Autonomic dysfunction in parkinsonian disorders: assessment and pathophysiology. J Neurol Neurosurg Psychiatry 84, 674-680 (2013)). The most notable loss of neurons occurs in the dopamine (DA) cells of the substantia nigra pars compacta (SNc), though neuronal loss also occurs in the locus coeruleus, nucleus basalis of Meynert, dorsal raphe nucleus and the dorsal motor nucleus of the vagus (N. Giguere, S. Burke Nanni, L. E. Trudeau, On Cell Loss and Selective Vulnerability of Neuronal Populations in Parkinson's Disease. Front Neurol 9, 455 (2018)). In addition to the loss of neurons, there is accumulation of misfolded pathologic α-synuclein that drives the pathogenesis of PD including the neuronal dysfunction and the ultimate loss of neurons (S. Mehra, S. Sahay, S. K. Maji, alpha-Synuclein misfolding and aggregation: Implications in Parkinson's disease pathogenesis. Biochim Biophys Acta Proteins Proteom 1867, 890-908 (2019); L. Stefanis, alpha-Synuclein in Parkinson's disease. Cold Spring Harb Perspect Med 2, a009399 (2012).). Treatments for Parkinson's Disease include the replacement of DA via L-DOPA, DA agonists and other agents to treat the non-motor symptoms. As the disease progresses, deep brain stimulation and other neurosurgical approaches are used to treat the side effects of DA replacement therapy. These treatments only address the symptomology, and there are no treatments that slow the progression or inhibit the underlying drivers of Parkinson's Disease pathogenesis. As such, treatments that can durably arrest Parkinson's Disease symptoms are urgently needed.

Irisinis a hormone formed by the cleavage of FNDC5. Since its discovery, irisin has been functionally associated with thermogenic programs (Bostrom et al. (2012) Nature 481:463-468; Oguri et al. (2020) Cell 182:563-577), bone remodeling (Colaianni et al. (2015) Proc. Natl. Acad. Sci. U.S.A. 112:12157-12162; Kim et al. (2018) Cell 175:1756-1768; Estell et al. (2020) eLife 9:e58172), and cognition (Wrann et al. (2013) Cell Metab. 18:649-659; Wrann (2015) Brain Plast. 1:55-61).

However, modulators of FNDC5 and biologically active fragments thereof, such as irisin, have not been previously used to treat neurodegenerative disease. There remains a need for the development of novel therapeutics to treat Parkinson's Disease and related diseases.

### SUMMARY OF THE INVENTION

The present invention is based, at least in part, on the discovery that increased expression of Fndc5 polypeptide or a biologically active fragment thereof increases irisin and has direct effects on α-synuclein (e.g., pathologic α-synuclein) in neurons, specifically that irisin can prevent the degeneration of dopaminergic (DA) neurons and motor deficits induced by α-synuclein (e.g., pathologic α-synuclein), which is useful in ameliorating a wide variety of α-synucleinopathies, such as Parkinson's disease, Lewy body dementia, multiple system atrophy (MSA), Alzheimer's disease or a neuroaxonal dystrophy. The methods and compositions disclosed herein may also be used to treat cancers characterized by or caused by an increase in the amount of or level of α-synuclein (e.g., pathologic α-synuclein) in cancer cells (e.g., melanoma cells), such as in a tumor or tumor microenvironment.

In one aspect, provided herein are methods of preventing or reducing degeneration of dopaminergic neurons, preventing or ameliorating at least one motor deficit (e.g., tremor at rest, such as a slight tremor in the hands or feet; rigidity (stiffness) of limbs, neck, or shoulders; difficulty balancing (postural instability); slowness of movement or gradual loss of spontaneous movement (bradykinesia); trouble standing after sitting; stiffness in the limbs, or moving more slowly) and/or preventing or ameliorating at least one symptom of cognitive dysfunction or dementia (e.g., confusion, poor motor coordination, loss of short-term or long-term memory, identity confusion, or impaired judgment) in a subject in need thereof, the method comprising administering to the subject an agent that is selected from the group consisting of: i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof, optionally wherein the biologically active Fndc5 fragment is irisin. In some embodiments, the subject is afflicted with an α-synucleinopathy, such as Parkinson's disease, Lewy body dementia, multiple system atrophy (MSA), Alzheimer's disease or a neuroaxonal dystrophy.

In some aspects, provided herein is a method of blocking the accumulation of or reducing the level or amount of α-synuclein (e.g., pathologic α-synuclein) in the cells of a subject in need thereof, the method comprising administering to the subject an agent that is selected from the group consisting of: i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof, optionally wherein the biologically active Fndc5 fragment is irisin.

The cells may be neurons, glia, cancer cells, or any cell in which α-synuclein (e.g., pathologic α-synuclein) can accumulate and cause a pathogenic response. The subject may be afflicted with a cancer characterized by an increase of α-synuclein (e.g., pathologic α-synuclein), or a cancer caused by an increase of α-synuclein (e.g., pathologic α-synuclein). In some embodiments, subject is afflicted with an α-synucleinopathy, such as Parkinson's disease, Lewy body dementia, multiple system atrophy (MSA), Alzheimer's disease or a neuroaxonal dystrophy.

Also provided herein are methods of treating or preventing Parkinson's disease, Lewy Body dementia, multiple system atrophy (MSA), Alzheimer's disease or a neuroaxonal dystrophy in a subject in need thereof, the method comprising administering to the subject an agent that is selected from the group consisting of: i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof, optionally wherein the biologically active Fndc5 fragment is irisin. The agent may be administered systemically, such as through intravenous or subcutaneous administration. The agent may be administered in a pharmaceutically acceptable formulation. The agent may be administered in a therapeutically effective amount to treat Parkinson's disease, Lewy Body dementia, multiple system atrophy (MSA), Alzheimer's disease or a neuroaxonal dystrophy. The agent may be administered at least once a day, at least one a week, or at least once a month. In some embodiments, the agent is administered to the subject for greater than a number of months equal to 3, 4, 5, 6, 7, 8, 9, 10, 11, 12, 13, 14, 15, 16, 17, 18, 19, 20, 21, 22, 23, or 24. In some embodiments, the agent is administered for the duration or remainder of the subject's life.

In some embodiments, the agent may be selected from the group consisting of:
a) a polypeptide comprising an amino acid sequence having at least 70% identity to the amino acid sequence of SEQ ID NO: 2, wherein said fragment lacks the C-terminal domain sequence of said FNDC5 polypeptide, and wherein said polypeptide has one or more of the biological activities of said FNDC5 polypeptide;
b) a polypeptide comprising an amino acid sequence having at least 70% identity to the amino acid sequence of residues 73-140 of the FNDC5 polypeptide of SEQ ID NO:2, wherein said polypeptide does not encode the C-terminal domain sequence of said FNDC5 polypeptide, and wherein said polypeptide has one or more of the biological activities of said FNDC5 polypeptide;
c) a polypeptide fragment of the FNDC5 polypeptide of SEQ ID NO: 2, wherein the fragment consists of a sequence of amino acids in between residues 1 and 150 of SEQ ID NO: 2, and wherein the fragment has one or more of the biological activities of said FNDC5 polypeptide; and
d) a polypeptide fragment of FNDC5 comprising an amino acid sequence having at least 70% identity to the amino acid sequence of a fragment of a FNDC5 polypeptide of SEQ ID NO: 4, 6 or 8, and wherein said fragment has one or more of the biological activities of said FNDC5 polypeptide.

In some embodiments, the polypeptide is fused to one or more heterologous polypeptides at its N-terminus and/or C-terminus. The polypeptide may comprise an amino acid modification, post-translational modification, and/or a heterologous an amino acid sequence, that stabilizes the polypeptide and/or increases its half-life. In some embodiments, the one or more heterologous polypeptides is an Fc domain or fragment thereof. In some embodiments, the agent is a nucleic acid that encodes an Fndc5 polypeptide or a biologically active fragment thereof, optionally wherein the Fndc5 polypeptide or biologically active fragment thereof is a polypeptide disclosed herein. The nucleic acid may be comprised within an expression vector (e.g., a viral expression vector, optionally wherein the viral expression vector is an adeno-associated viral (AAV) vector). Each dose of the viral expression vector may be at least 1×10² GC/kg particles, at least 1×10³ GC/kg particles, at least 1×10⁴ GC/kg particles, at least 1×10⁵ GC/kg particles, at least 1×10⁶ GC/kg particles, at least 1×107 GC/kg particles, at least 1×10⁸ GC/kg particles, at least 1×10⁹ GC/kg particles, at least 1×10¹⁰ GC/kg particles, at least 1×10¹¹ GC/kg particles, at least 1×10¹² GC/kg particles, at least 1×10¹³ GC/kg particles, at least 1×10¹⁴ GC/kg particles, at least 1×10¹⁵ GC/kg particles, at least 1×10¹⁶ GC/kg particles, at least 1×10¹⁷ GC/kg particles, at least 1×10¹⁸ GC/kg particles, at least 1×10¹⁹ GC/kg particles, at least 1×10²⁰ GC/kg particles, at least 1×10²¹ GC/kg particles, at least 1×10²² GC/kg particles, at least 1×10²³ GC/kg particles, at least 1×10²⁴ GC/kg particles, at least 1×10²⁵ GC/kg particles, at least 1×10²⁶ GC/kg particles, at least 1×10²⁷ GC/kg particles, at least 1×10²⁸ GC/kg particles, at least 1×10²⁶ GC/kg particles, at least 1×10²⁷ GC/kg particles, at least 1×10²⁸ GC/kg particles, at least 1×10²⁹ GC/kg particles, or at least 1×10³⁰ GC/kg particles. In some embodiments, the agent crosses the blood brain barrier (BBB). In some embodiments, the agent does not increase the levels of brain-derived neurotrophic factor (BDNF) in neurons in the subject over the course of treatment. The course of treatment may beat least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 months, or longer, or any range in between, inclusive, such as 12-24 months. The course of treatment may be the duration or remainder of the subject's life.

The method may further comprise administering conjointly to the subject an additional agent that increases the expression or activity of Fndc5 or a biologically active fragment thereof, optionally wherein the biologically active fragment of Fndc5 is irisin.

The subject may be a mammal, optionally the mammal is a rodent, a primate, or a human.

Also provided herein is a method of stratifying patients afflicted with a condition disclosed herein, the method comprising measuring the levels of α-synuclein in cells isolated from a subject afflicted with an α-synucleinopathy, and if the subject's cells measure above a specific amount of α-synuclein (e.g., pathogenic α-synuclein), administering to the subject an agent that is selected from the group consisting of: i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof, optionally wherein the biologically active Fndc5 fragment is irisin. In some embodiments, the specific amount of α-synuclein may be at least 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, or 500% increase in the amount of α-synuclein (e.g., pathogenic α-synuclein) when compared to a control sample (e.g., a biological sample from a patient not afflicted with a disorder or disease disclosed herein)

### BRIEF DESCRIPTION OF FIGURES

The patent of application file contains at least one drawing executed in color. Copies of this patent or patent application publication with color drawing(s) will be provided by the Office upon request and payment of the necessary fee.

FIG. 1A-FIG. 1F show that irisin protects neurons against α-synuclein PFF-induced neurotoxicity. FIG. 1A shows representative images of pS129-α-synuclein (green) in primary cortical neurons pre-incubated with indicated concentration of irisin for 1 hour, and further incubated with α-synuclein PFF (1 μg/ml) for 7 days. Nuclei are stained with DAPI (blue). Scale bar, 20 μm. FIG. 1B shows quantification of p-α-synuclein signals normalized with DAPI. Bars represent mean±s.e.m. One-way ANOVA followed by Tukey's post hoc test (n=3). FIG. 1C shows representative immunoblots of pS129-α-syn and α-syn in the Triton X-100-soluble and insoluble fraction from primary cortical neurons preincubated for 1 hour followed by sustained treatment with indicated concentration of irisin followed by incubation with α-syn PFF for 7 days. FIG. 1D shows quantification of levels of pS129-α-syn and α-syn in the Triton X-100-insoluble fraction normalized to β-actin shown in FIG. 1C. Bars represent mean±s.e.m. One-way ANOVA followed by Tukey's post hoc test (n=4). FIG. 1E shows cell death assay quantified from Hoechst and propidium iodide (PI) staining in primary cortical neurons treated for 1 hour followed by sustained treatment with indicated concentration of irisin and further incubated with α-syn PFF (5 μg/mL) for 14 days. Bars represent mean±SEM. Two-way ANOVA followed by Tukey's post hoc test (n=4). FIG. 1F shows cell death assay quantified from Hoechst and propidium iodide (PI) staining in primary cortical neurons preincubated 1 hour followed by sustained treatment with irisin (50 ng/mL) and further incubated with α-syn PFF (5 μg/mL) for 14 days as well as delayed treatment (1 day, 2 days, 4 days, and 7 days) after α-syn PFF treatment. Bars represent mean±s.e.m. One-way ANOVA followed by Tukey's post hoc test (n=4). *P<0.05, **P<0.005, ***P<0.0005.

FIG. 2A-FIG. 2F show that irisin increases the degradation of pathologic α-synuclein. FIG. 2A shows a schematic diagram of transmission of pathologic α-synuclein. Pathologic α-synuclein are released from the donor cell via exosomes and transmitted into recipient cells via endocytosis. FIG. 2B shows primary cortical neurons from WT embryos that were pretreated with 50 ng/ml irisin for 1 hour and further incubated with biotin-conjugated α-synuclein PFF (1 μg/ml) for 2 hours. The endocytosis of α-synuclein-biotin PFF in the endosome-enriched fraction was determined by immunoblotting using anti-streptavidin antibody. Bars represent mean±s.e.m. Student t-test (n=3) (ns, not significant). FIG. 2C shows primary cortical neurons from WT embryos that were pretreated with 50 ng/ml irisin for 1 hour and further incubated with biotin-conjugated α-synuclein PFF (1 g/ml) for 24 hours. The secreted α-synuclein in exosome was detected by western blot analysis (n=3). There was no significant difference by treatment of irisin. Bars represent mean±s.e.m. Student t-test (n=3). ***P<0.0005. FIG. 2D shows propagated α-synuclein PFF is degraded by lysosomes. Primary cortical neurons pretreated with PBS, NH₄Cl or MG132 were further incubated with biotin-conjugated α-synuclein PFF (1 μg/ml) for 12 hours. Twelve hours after changing to fresh medium, the intracellular biotin-conjugated α-synuclein PFF levels were determined by immunoblotting using anti-streptavidin antibody. Bars represent mean±s.e.m. One-way ANOVA followed by Tukey's post hoc test (n=3). ***P<0.0005. FIG. 2E shows that irisin promotes intracellular degradation of propagated α-synuclein PFF. Primary cortical neurons were pretreated with 50 ng/ml irisin for 1 hour and further incubated with biotin-conjugated α-synuclein PFF (1 μg/ml) for 12 hours. The levels of intracellular biotin-conjugated α-synuclein PFF were determined by immunoblotting using anti-streptavidin antibody 3, 6, or 12 hours after changing to fresh medium. Graph represents mean±s.e.m. Two-way ANOVA followed by Tukey's post hoc test (n=3). *P<0.05, ***P<0.0005. FIG. 2F shows degradation of pathologic α-synuclein PFF, but not endogenous α-synuclein by irisin. Primary cortical neurons were pretreated with 50 ng/ml irisin for 1 hour and further incubated with biotin-conjugated α-synuclein PFF (1 μg/ml) for 72 hours. The levels of pathologic α-synuclein and endogenous α-synuclein were determined by immunoblotting using anti-streptavidin and α-synuclein antibodies, respectively. Bars represent mean±s.e.m. Two-way ANOVA followed by Tukey's post hoc test (n=4). ND, not determined; ns, not significant, ***P<0.0005.

FIG. 3A-FIG. 3R show that irisin protects α-synuclein PFF-induced pathology in vivo. FIG. 3A shows a schematic diagram of in vivo experiments. Two-month-old WT mice were stereotaxically injected with PBS or α-synuclein PFF (5 μg/mouse) into the striatum. Two weeks after α-synuclein PFF injection, the mice were injected with AAV8-GFP or AAV8-Irisin-FLAG (1E10 G.C./mouse) via the tail vein. Six months after α-synuclein PFF injection, the mice were subjected to behavioral test (pole test and grip strength test), stereology and biochemical analysis. FIG. 3B shows representative TH and Nissl staining of SNpc DA neurons of PBS or α-synuclein PFF injected mice treated with AAV-GFP or AAV-irisin at 6 months after α-synuclein PFF or PBS injection. Scale bars, 400 μm. FIG. 3C and FIG. 3D shows stereological counts of (FIG. 3C) TH⁺ and (FIG. 3D) TH⁺Nissl⁺ cells. Data are mean±s.e.m. ***P<0.0005, two-way ANOVA followed by Tukey's post hoc test (n=5 mice per group). FIG. 3E shows representative immunoblots of TH, DAT, and β-actin in the ipsilateral striatum of injected mice. Quantification of TH and DAT levels in the striatum normalized to β-actin. Bars represent the mean±s.e.m.***P<0.0005, two-way ANOVA followed by Tukey's post hoc test (n=4). FIG. 3F shows representative photomicrograph of striatal sections stained for TH fiber density. FIG. 3G shows quantification of dopaminergic fiber densities in the striatum using Image J software (NIH). Bars represent mean±s.e.m. Two-way ANOVA followed by Tukey's post hoc test (n=6). FIG. 3H shows representative immunoblots of pS129-α-syn and α-syn in the detergent-soluble and insoluble fraction from the SNpc of injected mice. FIG. 3I shows quantification of pS129-α-syn and α-syn levels in the detergent-insoluble fraction normalized to β-actin. Bars represent mean±SEM. Two-way ANOVA followed by Tukey's post hoc test (n=3). FIG. 3J and FIG. 3K show that 180 days after intrastriatal α-syn PBS or PFF injection, pole test (FIG. 3J) and grip strength test (FIG. 3K) were performed. Data are the mean±s.e.m. *P<0.05, ***P<0.0005, two-way ANOVA followed by Tukey's post hoc test (n=12-13 mice per group). FIG. 3L shows dopamine concentrations in the striatum of PBS or α-syn PFF injected mice treated with AAV-GFP or AAV-Irisin determined by HPLC. Bars represent mean±SEM. Two-way ANOVA followed by Tukey's post hoc test. (n=7 mice per group). FIG. 3M-FIG. 3O shows (FIG. 3M) DOPAC, (FIG. 3N) HVA and (FIG. 3O) 3MT concentrations in the striatum of PBS or α-syn PFF injected mice treated with AAV-GFP or AAV-Irisin at 6 months after α-syn PFF or PBS injection measured by HPLC. Bars represent mean±s.e.m. Two-way ANOVA followed by Tukey's post hoc test. (n=7 mice per group). FIG. 3P and FIG. 3Q show DA turnover as determined by (FIG. 3P) (DOPAC+HVA)/DA and (FIG. 3Q) (DOPAC+3MT)/DA was calculated from the striatum. Bars represent mean±s.e.m. Two-way ANOVA followed by Tukey's post hoc test (n=7 mice per group). FIG. 3R shows that 180 days after intrastriatal α-syn PBS or PFF injection, grip strength test were performed. Data are the mean±s.e.m. Two-way ANOVA followed by Tukey's post hoc test (n=12-13 mice per group). *P<0.05, **P<0.005, ***P<0.0005.

FIG. 4A-FIG. 4G show that irisin reduces the α-syn levels. FIG. 4A shows schematic diagram of proteomic analysis. FIG. 4B and FIG. 4C show volcano plots of protein alterations. The proteins were quantified from primary cortical neurons with or without pre-incubation of irisin (50 ng/mL) and further incubated with α-syn PFF (1 μg/mL) for (FIG. 4B) 1 or (FIG. 4C) 4 days were analyzed for differentially expressed proteins in PFF- and irisin-treated cells. The cutoff used to select differentially expressed proteins was q-value<0.05. FIG. 4D and FIG. 4E show elative protein levels of (FIG. 4D) ApoE and (FIG. 4E) Snca in primary cortical neurons 1 and 4 d after PBS, α-syn PFF, or α-syn PFF with irisin administration analyzed by mass spec. Bars represent mean±SEM. Two-way ANOVA followed by Tukey's post hoc test (n=3). FIG. 4F shows representative immunoblots of pS129-α-syn, α-syn and α-syn-biotin in the detergent-insoluble and soluble fraction from cortical neurons 1 or 4 days after treatment. FIG. 4G shows quantification of α-syn-biotin and α-syn levels in the detergent-soluble fraction normalized to β-actin. Bars represent mean±SEM. Two-way ANOVA followed by Tukey's post hoc test (n=3). *P<0.05, **P<0.005, ***P<0.0005. ND, not determined; ns, not significant.

FIG. 5A-FIG. 5H show that irisin increases the degradation of pathologic α-syn. FIG. 5A and FIG. 5B show primary cortical neurons from WT embryos were pretreated with 50 ng/mL Irisin for 1 hour and further incubated with biotin-conjugated α-syn PFF (1 μg/mL) for 24 hours. The levels of α-syn-biotin and α-syn in the endolysosome-enriched fraction were determined by immunoblotting using anti-streptavidin and an anti-α-syn antibody, respectively. Rab7 is a marker for endosome, Lamp2 is a marker for lysosome, HSP60 is a marker for mitochondria, and α-tubulin is a marker for cytoplasm. Bars represent mean±SEM. Two-way ANOVA followed by Tukey's post hoc test (n=4). FIG. 5C shows that irisin promotes intracellular degradation of propagated α-syn PFF. Primary cortical neurons were pretreated with 50 ng/mL Irisin for 1 hour and further incubated with biotin-conjugated α-syn PFF (1 μg/mL) in the presence of 50 ng/mL irisin for 12 hours followed by media replacement with 50 ng/mL irisin not containing α-syn PFF. Intracellular biotin-conjugated α-syn PFF levels were determined by immunoblotting using anti-streptavidin antibody 3, 6, and 12 hours after changing to fresh medium containing 50 ng/mL irisin. Graph represents mean±SEM. Two-way ANOVA followed by Tukey's post hoc test (n=3). FIG. 5D and FIG. 5E show that propagated α-syn PFF is degraded by the lysosome. Primary cortical neurons were pretreated with 50 ng/mL Irisin for 1 hour and further incubated with biotin-conjugated α-syn PFF (1 μg/mL) for 12 hours, followed by the fresh medium or medium containing NH₄Cl was replaced for 3 hours. The levels of α-syn-biotin and α-syn were determined by immunoblotting using anti-streptavidin and α-syn antibodies, respectively. Graph represents mean±SEM. Two-way ANOVA followed by Tukey's post hoc test (n=3). FIG. 5F shows representative microscopic images of pS129-α-syn (green) in primary cortical neurons treated with α-syn PFF (1 μg/mL) for 4 days. Two days after α-syn PFF treatment, irisin and NH₄Cl were incubated for 2 days. DAPI (blue) is used for nuclei staining. (Scale bar, 20 m.) Quantification of p-α-syn signals was normalized with DAPI. Bars represent mean±SEM. One-way ANOVA followed by Tukey's post hoc test (n=3). FIG. 5G shows representative immunoblots of pS129-α-syn and α-syn in the detergent-soluble and insoluble fraction from primary cortical neurons incubated with α-syn PFF for 4 days followed by posttreated with irisin and NH₄Cl for 2 days. FIG. 5H shows quantification of pS129-α-syn and α-syn levels in the detergent-insoluble fraction normalized to β-actin. Bars represent mean±SEM. One-way ANOVA followed by Tukey's post hoc test (n=4). *P<0.05, **P<0.005, ***P<0.0005. ND, not determined; ns, not significant.

FIG. 6A-FIG. 6C show that irisin induces clearance of α-syn in SK-Mel2 melanoma cells. FIG. 6A shows that irisin induces clearance of α-synuclein aggregates in cultured SK-Mel2 and A375 melanoma cells in a dose-dependent manner. FIG. 6B shows that α-synuclein KO SK-Mel2 melanoma cells lost their irisin-mediated effects. FIG. 6C shows that immune-compromised mice implanted with A375 melanoma cells exhibited less tumor burden upon AAV8-irisin administration compared to AA8-GFP administrated control group, and the irisin-mediated reduction of tumor burden correlates with the reduction of aggregated α-synuclein levels in the tumor tissue.

FIG. 7A-FIG. 7D show blood-brain penetration of intravenously injected irisin. In FIG. 7A and FIG. 7B, two weeks after intrastriatal α-syn PFF injection, mice were injected with AAV8-GFP or AAV8-Irisin-FLAG (1E10 G.C./mouse) via the tail vein; six months after α-syn PFF injection, (FIG. 7A) irisin-FLAG levels in the plasma and (FIG. 7B) irisin mRNA expression in the liver were determined by ELISA and qPCR, respectively. Bars represent mean±s.e.m. Two-way ANOVA followed by Tukey's post hoc test. (n=6 mice per group). In FIG. 7C and FIG. 7D, C57BL/6 mice were intravenously (IV) injected with 1 mg/kg of purified irisin-His for 1 hour; the concentration of irisin in (FIG. 7C) plasma and (FIG. 7D) brain were measured by ELISA. Bars represent mean±s.e.m. Two-way ANOVA followed by Tukey's post hoc test. (n=3 mice per group). *P<0.05, **P<0.005, ***P<0.0005.

FIG. 8A-FIG. 8B show tandem mass spectrometry analysis of α-syn PFF-treated neurons. FIG. 8A and FIG. 8B show volcano plots of protein alterations. The proteins quantified from primary cortical neurons treated with PBS or α-syn PFF (1 μg/ml) for (FIG. 8A) 1 or (FIG. 8B) 4 days were analyzed for differentially expressed proteins in α-syn PFF treated cells.

### DETAILED DESCRIPTION OF THE INVENTION

The present invention is based in part on the discovery that modulators of Fndc5 and modulators of biologically active fragments thereof (e.g., irisin) can act to prevent or reduce degeneration of dopaminergic (DA) neurons preventing or ameliorating at least one motor deficit and/or preventing or ameliorating at least one symptom of cognitive dysfunction or dementia, such as to prevent or treat an α-synucleinopathy, such as Parkinson's disease, Lewy body dementia, Alzheimer's disease multiple system atrophy (MSA), or a neuroaxonal dystrophy. The present disclosure provides methods of using modulators of Fndc5 and modulators of biologically active fragments thereof (e.g., irisin) in such methods.

### I. Definitions

In order that the present invention may be more readily understood, certain terms are first defined. Additional definitions are set forth throughout the detailed description.

The articles “a” and “an” are used herein to refer to one or to more than one (i.e., to at least one) of the grammatical object of the article. By way of example, “an element” means one element or more than one element.

As used herein, the term “administering” a substance, such as a therapeutic entity to an animal or cell” is intended to refer to dispensing, delivering or applying the substance to the intended target. In terms of the therapeutic agent, the term “administering” is intended to refer to contacting or dispensing, delivering or applying the therapeutic agent to an animal by any suitable route for delivery of the therapeutic agent to the desired location in the animal, including delivery by either the parenteral or oral route, intramuscular injection, subcutaneous/intradermal injection, intravenous injection, buccal administration, transdermal delivery and administration by the intranasal or respiratory tract route.

The term “α-synucleinopathy” includes any neurodegenerative disease or disorder characterized by the accumulation of α-synuclein in cells or tissue (e.g., in neurons, nerve fibers, glial cells, cancer cells, and the like). In some embodiments, accumulation of α-synuclein is associated with multisystem neurodegeneration, and underlies a wide spectrum of clinical syndromes, movement disorders/parkinsonism (Parkinson's disease, pantothenate kinase-associated neurodegeneration), dementia (Parkinson's disease dementia, dementia with Lewy body), and autonomic dysfunction (pure autonomic failure, multiple system atrophy). Pathogenetically, they can arise from disturbances in the metabolism of α-synuclein (for example, increased synthesis or oligomer formation due to insufficient degradation). Therefore, as used herein, “α-synuclein” and “pathogenic α-synuclein” are interchangeable, and can refer to an increase in the amount of misfolded, phosphorylated and/or mutated α-synuclein, or to an increase in the overall levels of any form, including wildtype, of α-synuclein in cells or tissues to predispose or cause a pathogenic condition (e.g., a α-synucleinopathy or a cancer disclosed herein) in a subject. Although trace levels of phosphorylated α-synuclein are detectable in healthy brains, much of the α-synuclein accumulated within Lewy bodies in Parkinson's disease brains is phosphorylated on serine 129 (Ser-129). Therefore, increased levels of α-synuclein may refer not only to total levels of wild-type α-synuclein, but any mutated form of α-synuclein or phosphorylated form of α-synuclein, such as α-synuclein phosphorylated on serine 129 (Ser-129). Duplication, triplication and of the α-synuclein locus can cause an α-synucleinopathy (Giobbie-Hurder, A., et al. (2017). An immunogenic personal neoantigen vaccine for patients with melanoma. Nature, 547(7662), 217-221., Singleton, A. B., et al. (2003). α-synuclein locus triplication causes Parkinson's disease. Science (New York, N.Y.), 302(5646), 841). Polymorphisms in the α-synuclein gene can increase or decrease one's risk of developing α-synucleinopathy, based on the expression of α-synuclein (Pedersen, C. C., et al. (2021). A systematic review of associations between common SNCA variants and clinical heterogeneity in Parkinson's disease. NPJ Parkinson's disease, 7(1), 54). A subject with increased levels of α-synuclein includes patients whose measured levels of a pathogenic form of α-synuclein (such as a phosphorylated form) are increased, even when wild type levels are constant or decreasing. Increased levels of α-synuclein phosphorylated on serine 129 (Ser-129). Similarly, decreasing or reducing levels of α-synuclein include decreasing or reducing the amount of misfolded α-synuclein, mutated α-synuclein, wild type α-synuclein, or overall levels of α-synuclein. Similarly, blocking the accumulation of α-synuclein include blocking the increase of or maintaining the current amount of misfolded α-synuclein, mutated α-synuclein, wild type α-synuclein, or overall levels of α-synuclein.

A subject afflicted with an α-synucleinopathy may show degeneration of dopaminergic neurons, at least one motor deficit (e.g., tremor at rest, such as a slight tremor in the hands or feet; rigidity (stiffness) of limbs, neck, or shoulders; difficulty balancing (postural instability); slowness of movement or gradual loss of spontaneous movement (bradykinesia); trouble standing after sitting; stiffness in the limbs, or moving more slowly) and/or at least one symptom of cognitive dysfunction or dementia (e.g., confusion, poor motor coordination, loss of short-term or long-term memory, identity confusion, or impaired judgment).

α-synuclein is a highly conserved protein belonging to a multigene family that includes β-synuclein and γ-synuclein. α-synuclein is strongly expressed in neurons, highly enriched in presynaptic terminals, and transported predominantly in the slow component. Axonal transport abnormalities of α-synuclein have may cause or be associated with synucleinopathies. This is based on the observation that axonal α-synuclein pathology is pronounced in the disease and also on experimental evidence suggesting that α-synuclein may play a role in transport of presynaptic vesicles. As used herein, α-synucleinopathies also include age-related retardation in the normal transport of α-synuclein (K. A. Jellinger, Synucleinopathies, Encyclopedia of Movement Disorders, Academic Press, 2010, Pages 203-207). Non-limiting, representative examples of α-synucleinopathies include Parkinson's disease, Lewy body dementia, multiple system atrophy (MSA), Alzheimer's disease and a neuroaxonal dystrophy.

Parkinson's disease is a progressive neurodegenerative disease characterized by tremor and bradykinesia. A portion of patients with Parkinson's disease have a family history of the condition, and family-linked cases can result from genetic mutations in a group of genes—LRRK2, PARK2, PARK7, PINK1 or the SNCA gene.

Dementia with Lewy bodies (i.e., Lewy Body dementia) is characterized by the accumulation of aggregated α-synuclein protein in Lewy bodies, similar to Parkinson's disease and Parkinson's disease dementia. However, it is can also be accompanied by aggregation of amyloid-beta and tau proteins.

Alzheimer's disease is a progressive neurodegenerative disease most often associated with memory deficits and cognitive decline. The cardinal pathological features of the disease include the presence of amyloid plaques and neurofibrillary tangles. Dominantly inherited familial AD (FAD) can be caused by mutations in amyloid precursor protein (APP), presenilin 1 (PSEN1) or PSEN2 genes. Early onset Alzheimer's disease (EOAD) is defined by those affected before age 65; and though they are slightly more common than FAD cases. More common late onset AD (LOAD) is considered sporadic, although genetic risk factors have been identified, most notably apolipoprotein E gene (APOE). Pathology indicative, although not exhaustive, symptoms of Alzheimer's disease include moderate cortical atrophy that is most marked in multimodal association cortices and limbic lobe structures, extracellular amyloid plaques, Hirano bodies, granulovacuolar degeneration (GVD), cerebral amyloid angiopathy (CAA) and/or intracellular neurofibrillary tangles Greater than 50% of AD patients have alpha-synuclein pathology in addition to tau and amyloid beta. See Twohig, D., & Nielsen, H. M. (2019). α-synuclein in the pathophysiology of Alzheimer's disease. Molecular neurodegeneration, 14(1), 23.

Multiple system atrophy is a progressive brain disorder that affects movement and balance and disrupts the function of the autonomic nervous system. The autonomic nervous system controls body functions that are mostly involuntary, such as regulation of blood pressure. The most frequent autonomic symptoms associated with multiple system atrophy are a sudden drop in blood pressure upon standing (orthostatic hypotension), urinary difficulties, and erectile dysfunction in men. Two major types of multiple system atrophy have been described, which are distinguished by their major signs and symptoms at the time of diagnosis. In one type, known as MSA-P, a group of movement abnormalities called parkinsonism are predominant. These abnormalities include unusually slow movement (bradykinesia), muscle rigidity, tremors, and an inability to hold the body upright and balanced (postural instability). The other type of multiple system atrophy, known as MSA-C, is characterized by cerebellar ataxia, which causes problems with coordination and balance. This form of the condition can also include speech difficulties (dysarthria) and problems controlling eye movement.

Infantile neuroaxonal dystrophy (INAD) is a rare neurodegenerative disease characterized by regression of acquired motor skills, delayed motor coordination and eventual loss of voluntary muscle control. Biallelic mutations in the PLA2G6 gene have been identified as the most frequent cause of INAD.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. The names of the natural amino acids are abbreviated herein in accordance with the recommendations of IUPAC-IUB.

The term “binding” or “interacting” refers to an association, which may be a stable association, between two molecules, e.g., between a polypeptide encompassed by the present invention and a binding partner, due to, for example, electrostatic, hydrophobic, ionic and/or hydrogen-bond interactions under physiological conditions. Exemplary interactions include protein-protein, protein-nucleic acid, protein-small molecule, and small molecule-nucleic acid interactions.

The term “biological sample” when used in reference to a diagnostic assay is intended to include tissues, cells and biological fluids isolated from a subject, as well as tissues, cells and fluids present within a subject pleural fluid, pus, saliva, sebum, semen, serum, sweat, synovial fluid, tears, urine, vaginal lubrication, vitreous humor, vomit).

The terms “cancer” or “tumor” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., myelomas like multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), myeloma, multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma, or breast carcinoma. The epithelial cancers may be characterized in various other ways including, but not limited to, serous, endometrioid, mucinous, clear cell, Brenner, or undifferentiated. In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

The term “isolated polypeptide” refers to a polypeptide, in certain embodiments prepared from recombinant DNA or RNA, or of synthetic origin, or some combination thereof, which (1) is not associated with proteins that it is normally found within nature, (2) is isolated from the cell in which it normally occurs, (3) is isolated free of other proteins from the same cellular source, (4) is expressed by a cell from a different species, or (5) does not occur in nature.

The terms “label” or “labeled” refer to incorporation or attachment, optionally covalently or non-covalently, of a detectable marker into a molecule, such as a polypeptide. Various methods of labeling polypeptides are known in the art and may be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for secondary antibodies, metal binding domains, epitope tags). Examples and use of such labels are described in more detail below. In some embodiments, labels are attached by spacer arms of various lengths to reduce potential steric hindrance.

In some embodiments, the term “neuronal degeneration” or “neuron degeneration” includes any loss of neuronal cells in specified regions of the nervous system. The loss may be rapid, or it may be slow and progressive. Progressive loss of neural tissues includes death of neurons over a period of time. Degeneration may be the results of inability of the neurons to self-regenerate after neurodegenerative cell death or severe damage that occurs to the neural tissue. Loss of neurons in a single subject may be qualified in any number of ways, including comparison of neuronal number or density comparison to a population average based on age or other demographics. As another example, loss of neuronal number or density can be qualified as an initial measurement of neuronal number or density, and a lower number or loss of density at a second measurement in time can indicate neuronal degeneration. Alternatively, degeneration of neurons may be quantified by measurement of metabolites or striatal DA, such as 3,4-dihydroxyphenylacetic acid (DOPAC).

As used herein, “pathogenic α-synuclein” can refer to an increase in the amount of misfolded and/or mutated α-synuclein, or to an increase in the overall levels of any form, including wild type, of α-synuclein in cells or tissues to predispose or cause a pathogenic condition (e.g., a α-synucleinopathy or a cancer disclosed herein) in a subject. Although trace levels of phosphorylated α-synuclein are detectable in healthy brains, much of the α-synuclein accumulated within Lewy bodies in Parkinson's disease brains is phosphorylated on serine 129 (Ser-129). Therefore, increased levels of α-synuclein may refer not only to total levels of wild-type α-synuclein, but any mutated form of α-synuclein, such as α-synuclein phosphorylated on serine 129 (Ser-129).

The “tumor microenvironment” is an art-recognized term and refers to the cellular environment in which the tumor exists, and includes, for example, interstitial fluids surrounding the tumor, surrounding blood vessels, immune cells, other cells, fibroblasts, signaling molecules, and the extracellular matrix.

In some embodiments, the term “subject” refers to a mammalian subject, such as a rodent, primate, or human.

The term “treatment,” as used herein, is defined as the application or administration of a therapeutic agent to a patient, or application or administration of a therapeutic agent to an isolated tissue or cell line from a patient, who has a disease or disorder, a symptom of a disease or disorder or a predisposition toward a disease or disorder, with the purpose of curing, healing, alleviating, relieving, altering, remedying, ameliorating, improving or affecting the disease or disorder, the symptoms of disease or disorder or the predisposition toward a disease or disorder. A therapeutic agent includes, but is not limited to, polypeptides, small molecules, peptides, peptidomimetics, nucleic acid molecules, antibodies, ribozymes, siRNA molecules, and sense and antisense oligonucleotides described herein

As used herein, the terms “Fndc5” and “Frcp2” refer to fibronectin type III domain containing 5 protein and are intended to include fragments, variants (e.g., allelic variants) and derivatives thereof. The nucleotide and amino acid sequences of mouse Fndc5, which correspond to GenBank Accession number NM_027402.3 and NP_081678.1 respectively, are set forth in SEQ ID NOs: 1 and 2. At least three splice variants encoding distinct human Fndc5 isoforms exist (isoform 1, NM_001171941.2, NP_001165412.1; isoform 2, NM_153756.2, NP_715637.1; and isoform 3, NM_001171940.1, NP_001165411). The nucleic acid and polypeptide sequences for each isoform is provided herein as SEQ ID NOs: 3-8, respectively. Nucleic acid and polypeptide sequences of FNDC5 orthologs in organisms other than mice and human are well-known and include, for example, chimpanzee FNDC5 (XM_003949350.1, XP_003949399.1, XM_001155446.3, and XP_001155446.3), monkey FNDC5 (XM_001098747.2 and XP_001098747.2), worm FNDC5 (XM_544428.4 and XP_544428.4), rat FNDC5 (XM_002729542.3 and XP_002729588.2), chicken FNDC5 (XM_417814.2; XP_417814.2), and zebrafish FNDC5 (XM_001335368.1; XP_001335404.1). In addition, numerous anti-Fndc5 antibodies having a variety of characterized specificities and suitabilities for various immunochemical assays are commercially available and well-known in the art, including antibody LS-C486450 from Lifespan Biosciences, antibodies AG-25B-0027 and -0027B from Adipogen, antibody HPA051290 from Atlas Antibodies, antibodies PAN576Hu01, Hu02, MuOl, and Mu02 from Uscn Lifesciences, antibodies OACD03594 and OACD03595 from Aviva Systems Biology, antibody orb39441 from Biorbyt, antibody ab93373 from Abcam, antibody NBP2-14024 from Novus Biologicals, antibodies 509549 and 044959 from United States Biological, antibody ABCA2332953 from Abgent, and the like.

In some embodiments, fragments of Fndc5 having one or more biological activities of the full-length Fndc5 protein are described and employed. Such fragments can comprise or consist of at least one fibronectin domain of an Fndc5 protein without containing the full-length Fndc5 protein sequence. In some embodiments, Fndc5 fragments can comprise or consist of a signal peptide, extracellular, fibronectin, hydrophobic, and/or C-terminal domains of an Fndc5 protein without containing the full-length Fndc5 protein sequence. As further indicated in the Examples, Fndc5 orthologs are highly homologous and retain common structural domains well-known in the art. In other embodiments, the term “irisin” refers to the fragment representing residues 29 or 30 to 140 of SEQ ID NO: 2 or the corresponding residues in an FNDC5 ortholog thereof.

TABLE 1
SEQ ID NO: 1 Mouse Fndc5 cDNA Sequence
atg ccc ccagggccgtgcgcctggccg ccc cgcgccgcgctccgcctgtggctaggctgcg
tctgcttcgcgctg gtg cag gcggacagc ccc tcagcccct gtg aac gtg acc gt
ccggcacctcaaggccaactctgcc gtg gtcagctgg gat gtcctg gag gat gaa gt
g gtcattggctttgccatctct cag cagaagaag gat gtg cggatgctccggttcatt
cag gag gtgaac acc accacccggtcctgcgctctctgggacctg gag gaggac aca
gaa tat atcgtccat gtg cag gccatctccatc cag gga cag agcccagccagt g
ag cct gtg ctcttcaagacc ccacgc gag gctgaaaagatggcctcaaagaacaaa ga
t gag Gtg acc atgaag gagatggggaggaac cag cagctgcgaacg (ggg) gag g
tg ctgatcattgtt gtg gtcctcttcatgtgggcaggtgttatagctctcttctgccgc ca
g tat gat atcAtcaaggacaacgag ccc aataacaacaag gag aaa acc aagagcg
catcagaa acc agc Aca ccg gag catcag ggtgggggtctcctccgcagcaagatatg
a
SEQ ID NO: 2 Mouse Fndc5 Amino Acid Sequence
M P P G P C A W P P R A A L R L W L G C V C F A L V Q A D S P S A
P V N V T V R H L K A N S A V V S W D V L E D E V V I G F A I S Q
Q K K D V R M L R F I Q E V N T T T R S C A L W D L E E D T E Y I
V H V Q A I S I Q G Q S P A S E P V L F K T P R E A E K M A S K N
K D E V T M K E M G R N Q Q L R T G E V L I I V V V L F M W A G V
I A L F C R Q Y D I I K D N E P N N N K E K T K S A S E T S T P E
H Q G G G L L R S K I
SEQ ID NO: 3 Human Fndc5 (isoform 1) cDNA Sequence

| | |
|---|---|
| 1 | atgctgcgcttcatccaggaggtgaacaccaccacccgctcatgtgccctctgggacctg |
| 61 | gaggaggatacggagtacatagtccacgtgcaggccatctccattcagggccagagccca |
| 121 | gccagcgagcctgtgctcttcaagaccccgcgtgaggctgagaagatggcctccaagaac |
| 181 | aaagatgaggtaaccatgaaagagatggggaggaaccaacagctgcggacaggcgaggtg |
| 241 | ctgatcatcgtcgtggtcctgttcatgtgggcaggtgtcattgccctcttctgccgccag |
| 301 | tatgacatcatcaaggacaatgaacccaataacaacaaggaaaaaaccaagagtgcatca |
| 361 | gaaaccagcacaccagagcaccagggggggggcttctccgcagcaaggtgagggcaaga |
| 421 | cctgggcctggggggccaccctgtgcctcatgctctggt aa |

SEQ ID NO: 4 Human Fndc5 (isoform 1) Amino Acid Sequence

| | |
|---|---|
| 1 | mlrfiqevntttrscalwdleedteyivhvqaisiqgqspasepvlfktpreaekmaskn |
| 61 | kdevtmkemgrnqqlrtgevliivvvlfmwagvialfcrqydiikdnepnnnkektksas |
| 121 | etstpehqgggllrskvrarpgpgwatlclmlw |

SEQ ID NO: 5 Human Fndc5 (isoform 2) cDNA Sequence

| | |
|---|---|
| 1 | atgctgcgcttcatccaggaggtgaacaccaccacccgctcatgtgccctctgggacctg |
| 61 | gaggaggatacggagtacatagtccacgtgcaggccatctccattcagggccagagccca |
| 121 | gccagcgagcctgtgctcttcaagaccccgcgtgaggctgagaagatggcctccaagaac |
| 181 | aaagatgaggtaaccatgaaagagatggggaggaaccaacagctgcggacaggcgaggtg |
| 241 | ctgatcatcgtcgtggtcctgttcatgtgggcaggtgtcattgccctcttctgccgccag |
| 301 | tatgacatcatcaaggacaatgaacccaataacaacaaggaaaaaaccaagagtgcatca |
| 361 | gaaaccagcacaccagagcaccagggcggggggcttctccgcagcaagatatga |

SEQ ID NO: 6 Human Fndc5 (isoform 2) Amino Acid Sequence

| | |
|---|---|
| 1 | mlrfiqevntttrscalwdleedteyivhvqaisiqgqspasepvlfktpreaekmaskn |
| 61 | kdevtmkemgrnqqlrtgevliivvvlfmwagvialfcrqydiikdnepnnnkektksas |
| 121 | etstpehqgggllrski |

SEQ ID NO: 7 Human Fndc5 (isoform 3) cDNA Sequence

| | |
|---|---|
| 1 | atgctgcgcttcatccaggaggtgaacaccaccacccgctcatgtgccctctgggacctg |
| 61 | gaggaggatacggagtacatagtccacgtgcaggccatctccattcagggccagagccca |
| 121 | gccagcgagcctgtgctcttcaagaccccgcgtgaggctgagaagatggcctccaagaac |
| 181 | aaagatgaggtaaccatgaaagagatggggaggaaccaacagctgcggacaggcgaggtg |
| 241 | ctgatcatcgtcgtggtcctgttcatgtgggcaggtgtcattgccctcttctgccgccag |
| 301 | tatgacatcattgaagcgtg a |

SEQ ID NO: 8 Human Fndc5 (isoform 3) Amino Acid Sequence

| | |
|---|---|
| 1 | mlrfiqevntttrscalwdleedteyivhvqaisiqgqspasepvlfktpreaekmaskn |
| 61 | kdevtmkemgrnqqlrtgevliivvvlfmwagvialfcrqydiiea |

SEQ ID NO: 9 Chicken Fndc5 cDNA Sequence

| | |
|---|---|
| 1 | atggagaagaacagggacggccgcggcccccctggtgtccatctggggatggagaaggaa |
| 61 | gatgatttagagcccggtgacacgccggggctgcgcgaagccctggtggcgagatgtcac |
| 121 | cgctgccgcgcacccgccgggggtctcaccgggacgggccccgtttgctccttccggcga |
| 181 | tggggagcggtccgggccgagggctcccggtcccgcctgggggaaactgaggcagacggc |
| 241 | ggggccgggcggggcgggggccgagccgcccccgggccgggggagggaccggagcggggc |
| 301 | tgcccagcgctgcagcgggcggagccggggctcggcggggccgcctcccggccgagccga |
| 361 | gccgaaccgagccgcgctgccgagggccgccgagcccgcagccgcccccggccgaaccgg |
| 421 | gcggccccgccggttccgggccccggagctctccgcggtgctgaacggcgccgccgcgcc |
| 481 | cgcgggacgccggccccggagcggctcggccccggcgcggcgcggcgggccgcgggggga |
| 541 | tggagcccttcctgggctgcaccggcgccgcgctcctgctctgctttcagctacgccggt |
| 601 | ctgcggccggtggaggcagacagcccttcggctccggtcaatgtcacagtcaaacacctg |
| 661 | aaggccaactcagctgtagtgacttgggacgttctggaggatgaagttgtcattggattt |
| 721 | gccatttcccagcagaagaaggacgtgcggatgctgcgcttcatccaggaggtgaacacc |
| 781 | accacccgctcctgtgccctctgggacctagaggaggacactgagtacattgtgcatgtc |
| 841 | caggccatcagcatccaaggccagagccctgccagtgagccagtcctcttcaagaccccc |
| 901 | agggaagctgagaaactggcttctaaaaataaagatgaggtgacaatgaaggagatggcg |
| 961 | aagaaaaaccaacagctgcgcgcaggggaaatactcatcattgtggtggtgttgtttatg |
| 1021 | tgggcaggggtgatcgccctgttctgcaggcagtacgacatcatcaaagacaacgagccg |
| 1081 | aacaacagcaaggagaaagccaagagcgcctcagagaacagcacccccgagcaccagggt |
| 1141 | ggggggctgctccgcagcaagttcccaaaaaacaaaccctcagtgaacatcattgaggca |
| 1201 | taa |

SEQ ID NO: 10 Chicken Fndc5 Amino Acid Sequence

| | |
|---|---|
| 1 | meknrdgrgppgvhlgmekeddlepgdtpglrealvarchrcrapaggltgtgpvcsfrr |
| 61 | wgavraegsrsrlgeteadggagrgggraapgpgegpergcpalqraepglggaasrpsr |
| 121 | aepsraaegrrarsrprpnraappvpgpgalrgaerrrrargtpaperlgpgaarraagg |
| 181 | wspswaapaprscsafsyaglrpveadspsapvnvtvkhlkansavvtwdvledevvigf |
| 241 | aisqqkkdvrmlrfiqevntttrscalwdleedteyivhvqaisiqgqspasepvlfktp |
| 301 | reaeklasknkdevtmkemakknqqlrageiliivvvlfmwagvialfcrqydiikdnep |
| 361 | nnskekaksasenstpehqgggllrskfpknkpsvniiea |

SEQ ID NO: 11 Zebrafish Fndc5 cDNA Sequence

| | |
|---|---|
| 1 | atgagttcttacagtttggcagctccagtgaatgtgtccatcagggatctgaagagcagc |
| 61 | tcagccgtggtgacatgggacacgccagacggagagccagtcatcggcttcgccatcaca |
| 121 | caacagaagaaagatgtccgcatgctgcgctttattcaagaagtgaacaccaccacgcgg |
| 181 | agctgtgcattgtgggatctggaagctgatacggattacattgtgcacgttcagtctatc |
| 241 | agcatcagcggggcgagtcctgttagtgaagctgtgcacttcaagaccccgacagaagtt |
| 301 | gaaacacaggcctccaagaacaaagacgaggtgacgatggaggaggtcgggccgaacgct |
| 361 | cagctcagggccggagagttcatcattattgtggtggtcctcatcatgtgggcaggtgtg |
| 421 | atcgcactattctgccgtcagtatgacatcattaaagacaacgaaccaaacaataacaag |
| 481 | gataaagccaagaactcgtctgaatgcagcactccagagcacacgtcaggtggcctgctg |
| 541 | cgcagtaaggtataa |

SEQ ID NO: 12 Zebrafish Fndc5 Amino Acid Sequence

| | |
|---|---|
| 1 | mssyslaapvnvsirdlksssavvtwdtpdgepvigfaitqqkkdvrmlrfiqevntttr |
| 61 | scalwdleadtdyivhvqsisisgaspvseavhfktptevetqasknkdevtmeevgpna |
| 121 | qlragefiiivvvlimwagvialfcrqydiikdnepnnnkdkaknssecstpehtsggll |
| 181 | rskv |

SEQ ID NO: 13 Fragment of Murine Fndc5 Nucleic Acid
Sequence that encodes amino acid residues 29-140 of
murine Fndc5

| | |
|---|---|
| 104 | gacagcccctcagcccc |
| 121 | tgtgaacgtgaccgtccggcacctcaaggccaactctgccgtggtcagctgggatgtcct |
| 181 | ggaggatgaagtggtcattggctttgccatctctcagcagaagaaggatgtgcggatgct |
| 241 | ccggttcattcaggaggtgaacaccaccacccggtcctgcgctctctgggacctggagga |
| 301 | ggacacagaatatatcgtccatgtgcaggccatctccatccagggacagagcccagccag |
| 361 | tgagcctgtgctcttcaagaccccacgcgaggctgaaaagatggcctcaaagaacaaaga |
| 421 | tgaggtgaccatgaaggag |

SEQ ID NO: 14 Murine Fndc5 (residues 29-140)
DSPSAPVNVTVRHLKANSAVVSWDVLEDEVVIGFAISQQKKDVRMLRFIQEVNTTTRSCALWDLE
EDTEYIVHVQAISIQGQSPASEPVLFKTPREAEKMASKNKDEVTMKE
SEQ ID NO: 15 Fragment of Human Fndc5 Nucleic Acid Sequence

| | |
|---|---|
| 161 | gacagtccctcagccccagt |
| 181 | gaacgtcaccgtcaggcacctcaaggccaactctgcagtggtgagctgggatgttctgga |
| 241 | ggatgaggttgtcatcggatttgccatctcccagcagaagaaggatgtgcggatgctgcg |
| 301 | cttcatccaggaggtgaacaccaccacccgctcatgtgccctctgggacctggaggagga |
| 361 | tacggagtacatagtccacgtgcaggccatctccattcagggccagagcccagccagcga |
| 421 | gcctgtgctcttcaagaccccgcgtgaggctgagaagatggcctccaagaacaaagatga |
| 481 | ggtaaccatgaaagag |

It will be appreciated that specific sequence identifiers (SEQ ID NOs) have been referenced throughout the specification for purposes of illustration and should therefore not be construed to be limiting. Any marker encompassed by the present invention, including, but not limited to, the markers described in the specification and markers described herein, are well-known in the art and may be used in the embodiments encompassed by the present invention.

### II. Screening Assays

Methods (also referred to herein as a “screening assay”) are provided for identifying enhancers of the expression or activity of Fndc5 or a biologically active fragment thereof (e.g., irisin), i.e., candidate or test compounds or agents (e.g., polypeptides, peptides, peptidomimetics, small molecules (organic or inorganic) or other drugs), which promote or enhance expression or activity of Fndc5 or a biologically active fragment thereof (e.g., irisin). Compounds identified using assays described herein may be useful for modulating Fndc5 or a biologically active fragment thereof (e.g., irisin), e.g., increasing Fndc5 or irisin expression or activity. Thus, these compounds would be useful for treating or preventing an α-synucleinopathy as administration of Fndc5 or a biologically active fragment thereof (e.g., irisin) to a subject having an α-synucleinopathy can improve symptoms. Additionally, these compounds would be useful for treating or preventing caners caused by or characterized by α-synuclein as administration of Fndc5 or a biologically active fragment thereof (e.g., irisin) to a subject having an a cancers caused by or characterized by α-synuclein can improve symptoms.

These assays are designed to identify agents that replicate the function of Fndc5 or a biologically active fragment thereof (e.g., irisin), bind to or interact with such a protein, or bind to or interact with other intracellular or extracellular proteins that interact with such a protein. Such compounds may include, but are not limited to peptides, antibodies, nucleic acid molecules, siRNA molecules, or small organic or inorganic compounds. Such compounds may also include other cellular proteins.

Agents identified via assays such as those described herein may be useful, for example, for increasing expression or activity of Fndc5 or a biologically active fragment thereof (e.g., irisin), or activity-induced gene expression and/or physiology in neurons and/or cancerous tissues, such as tissues from cancers that are characterized by or caused by an increased level of α-synuclein, or, for example, maintaining integrity or decreasing degradation of neuronal cells. Thus, these compounds would be useful for treating or preventing an α-synucleinopathy. In some embodiments, increased activity or expression of Fndc5 or a biologically active fragment thereof (e.g., irisin) is sufficiently effective to treat or prevent an α-synucleinopathy. For example, a partial agonist or an agonist administered in a dosage or for a length of time to increase expression or activity of Fndc5 or a biologically active fragment thereof (e.g., irisin) would act to lower levels of pathological α-synuclein in tissues.

In one embodiment, the present invention provides assays for screening candidate or test compounds which are substrates of or interact with Fndc5 or a biologically active fragment thereof (e.g., irisin). In another embodiment, the present invention provides assays for screening candidate or test compounds which bind to or modulate the activity of Fndc5 or a biologically active fragment thereof (e.g., irisin). In still another embodiment, the present invention provides assays for screening candidate Fndc5 or a biologically active fragment thereof (e.g., irisin) having desired functional characteristics. The test agents encompassed by the present invention may be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide or peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries may be found in the art, for example in: DeWitt et al. (1993) Proc. Natl. Acad Sci. U.S.A. 90:6909; Erb et al. (1994) Proc. Natl. Acad Sci. USA 91:11422; Zuckermann et al. (1994). J. Med Chem. 37:2678; Cho et al. (1993) Science 261:1303; Carrell et al. (1994) Angew. Chem. Int. Ed Engl. 33:2059; Carell et al. (1994) Angew. Chem. Int. Ed Engl. 33:2061; and in Gallop et al. (1994) J. Med Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad Sci. 87:6378-6382); (Felici (1991) J. Mol. Biol. 222:301-310); (Ladner supra.).

In one embodiment, an assay is a cell-based assay in which a cell, such as a neuronal cell or a cancer cell, is contacted with a test agent, such as an Fndc5 or a biologically active fragment thereof (e.g., irisin), and the ability of the test compound to prevent or reduce degeneration of neurons, preventing or ameliorating at least one motor deficit and/or preventing or ameliorating at least one symptom of cognitive dysfunction or dementia to treat or prevent an α-synucleinopathy, or reduce the level or amount of α-synuclein in the cells of a subject, is determined. Determining the ability of the test agent to perform the functions discussed may be accomplished by monitoring biomarkers described herein, for example, biopsy, biomarker expression, physical assays, and the like.

The ability of the test agent to modulate the binding of Fndc5 or a biologically active fragment thereof (e.g., irisin) to a substrate may also be determined. Determining the ability of the test agent to modulate such binding may be accomplished, for example, by coupling the substrate with a radioisotope or enzymatic label such that binding of the substrate to Fndc5 or a biologically active fragment thereof (e.g., irisin) may be determined by detecting the labeled substrate in a complex. The Fndc5 or a biologically active fragment thereof (e.g., irisin) may also be coupled with a radioisotope or enzymatic label to monitor the ability of a test agent to modulate binding to the substrate in a complex. Determining the ability of the test agent to bind Fndc5 or a biologically active fragment thereof (e.g., irisin) may be accomplished, for example, by coupling the agent with a radioisotope or enzymatic label such that binding of the agent to Fndc5 or a biologically active fragment thereof (e.g., irisin) may be determined by detecting the labeled agent in a complex. For example, such agents may be labeled with ¹²⁵I, ³⁵S, ¹⁴C, or ³H, either directly or indirectly, and the radioisotope detected by direct counting of radio emmission or by scintillation counting. Agents can further be enzymatically labeled with, for example, horseradish peroxidase, alkaline phosphatase, or luciferase, and the enzymatic label detected by determination of conversion of an appropriate substrate to product.

It is also within the scope of the present invention to determine the ability of an agent to interact with Fndc5 or a biologically active fragment thereof (e.g., irisin), with or without the labeling of any of the interactants. For example, a microphysiometer may be used to detect the interaction without labeling any component (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS).

In another embodiment, modulators of expression or activity of Fndc5 or a biologically active fragment thereof (e.g., irisin) may be identified in a method wherein a cell is contacted with a candidate agent, such as an Fndc5 or a biologically active fragment thereof (e.g., irisin), and the expression of mRNA or protein of Fndc5 or a biologically active fragment thereof (e.g., irisin) in the cell is determined. The level of expression of mRNA or protein of Fndc5 or a biologically active fragment thereof (e.g., irisin) in the presence of the candidate agent is compared to that in the absence of the candidate agent. When expression of mRNA or protein Fndc5 or a biologically active fragment thereof (e.g., irisin) is greater (statistically significantly greater) in the presence of the candidate agent than in its absence, the candidate agent is identified as a stimulator of mRNA or protein expression of Fndc5 or a biologically active fragment thereof (e.g., irisin). The level of mRNA or protein expression of Fndc5 or a biologically active fragment thereof (e.g., irisin) in the cells may be determined by methods described herein for detecting mRNA or protein of Fndc5 or a biologically active fragment thereof (e.g., irisin).

In some embodiments, assays described herein may be conducted in cell-free formats using known components of gene expression of Fndc5 or a biologically active fragment thereof (e.g., irisin). It may be desirable to immobilize certain components of the assay, such as the Fndc5 or a biologically active fragment thereof (e.g., irisin) and such embodiments may benefit from the use of well-known adaptations for biomolecule immobilization, such as the use of microtitre plates, beads, test tubes, micro-centrifuge tubes in combination with derivatizable moieties, such as fusion protein domains, biotinylation, antibodies, and the like.

The present invention further pertains to novel agents identified by the above-described screening assays. Accordingly, it is within the scope of this invention to further use an agent identified as described herein in an appropriate animal model.

Any of the compounds, including, but not limited to, compounds, such as those identified in the foregoing assay systems, may be tested for a compound capable of ameliorating a condition disclosed herein, comprising the ability of the compound to modulate nucleic acid expression or polypeptide expression and/or activity of Fndc5 or a biologically active fragment thereof (e.g., irisin), thereby identifying a compound capable of ameliorating the condition. Cell-based and animal model-based assays for the identification of compounds exhibiting such an ability to ameliorate the condition (e.g., treat or prevent a muscular dystrophy) described herein.

In one aspect, cell-based systems, as described herein, may be used to identify agents such as a nucleic acid encoding an Fndc5 or a biologically active fragment thereof (e.g., irisin) that modulate Fndc5 polypeptide expression or Fndc5 polypeptide activity or treat a cancer that are characterized by or caused by an increased level of α-synuclein or an α-synucleinopathy. For example, such cell systems may be exposed to an agent at a sufficient concentration and for a time sufficient to elicit such an amelioration of disease symptoms in the exposed cells. After exposure, the cells may be examined to determine whether one or more of the disease phenotypes has been altered to resemble a more normal or more wild type phenotype.

In addition, animals or animal-based disease systems, such as those described herein, may be used to identify such agents. Such animal models may be used as test substrates for the identification of drugs, pharmaceuticals, therapies, and interventions which may be effective in modulating Fndc5 or a biologically active fragment thereof (e.g., irisin), such as to treat or prevent a cancer that are characterized by or caused by an increased level of α-synuclein or an α-synucleinopathy.

Additionally, gene expression patterns may be utilized to assess the ability of a compound to modulate expression or activity of Fndc5 or a biologically active fragment thereof (e.g., irisin). Thus, these compounds would be useful for treating, preventing, or assessing a cancer that are characterized by or caused by an increased level of α-synuclein or an α-synucleinopathy. For example, the expression pattern of one or more genes may form part of a “gene expression profile” or “transcriptional profile” which may be then be used in such an assessment. “Gene expression profile” or “transcriptional profile,” as used herein, includes the pattern of mRNA expression obtained for a given tissue or cell type under a given set of conditions. Gene expression profiles may be generated, for example, by utilizing a differential display procedure, Northern analysis and/or RT-PCR. Gene expression profiles may be characterized for known states within the cell- and/or animal-based model systems. Subsequently, these known gene expression profiles may be compared to ascertain the effect a test compound has to modify such gene expression profiles, and to cause the profile to more closely resemble that of a more desirable profile.

Additionally, also provided herein are methods of screening subjects by measuring or calculating the amount or level of α-synuclein in the cells of a subject to determine if the subject would benefit from a treatment method described herein. In some embodiments, provided herein are method of administrating of an agent selected from the group consisting of i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof if cells within the subject exhibit increases levels of α-synuclein. α-synuclein may be measured by any method know in the art. For example, a biological sample may be taken from a patient. Samples may be obtained by any means known in the art. Samples may also be taken directly from the nervous system, the tumor, or tumor microenvironment.

Additionally, the assays described herein may include measuring α-synuclein post isolated from cells. These may be conducted in cell-free formats using known components of gene expression of α-synuclein. It may be desirable to immobilize certain components of the assay and such embodiments may benefit from the use of well-known adaptations for biomolecule immobilization, such as the use of microtitre plates, beads, test tubes, micro-centrifuge tubes in combination with derivatizable moieties, such as fusion protein domains, biotinylation, antibodies, and the like. Gene or nucleic acid expression patterns may also be utilized to assess the levels of α-synuclein in a cell.

A detection method encompassed by the present invention may be used to detect mRNA, protein, or genomic DNA of α-synuclein or a pathogenic form of α-synuclein in a biological sample in vitro, as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody may be labeled with a radioactive marker whose presence and location in a subject may be detected by standard imaging techniques.

Antibodies directed against a pathogenic form of α-synuclein or α-synuclein may also be used in disease diagnostics and prognostics. Such antibodies are well-known in the art (see, for example, antibody ab138501 or ab212184 from Abcam, antibody Cat #32-8100 or Cat #MA1-90346 from ThermoFisher. Antibodies may be sourced from The Michael J. Fox Foundation. In addition, such diagnostic methods, may be used to detect abnormalities in the level of such polypeptide expression, or abnormalities in the structure and/or tissue, cellular, or subcellular location of such polypeptides. Structural differences may include, for example, differences in the size, electronegativity, or antigenicity of the mutant polypeptide relative to the normal polypeptide. Protein from the tissue or cell type to be analyzed may easily be detected or isolated using techniques that are well-known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, New York), which is incorporated herein by reference in its entirety.

This may be accomplished, for example, by immunofluorescence techniques employing a fluorescently labeled antibody (see below) coupled with light microscopic, flow cytometric, or fluorimetric detection. The antibodies (or fragments thereof) useful according to the present invention may, additionally, be employed histologically, as in immunofluorescence or immunoelectron microscopy, for in situ detection of Fndc5 or a biologically active fragment thereof (e.g., irisin). In situ detection may be accomplished by removing a histological specimen from a subject, and applying thereto a labeled antibody of the present invention. The antibody (or fragment) is may be applied by overlaying the labeled antibody (or fragment) onto a biological sample. Through the use of such a procedure, it is possible to determine not only the presence of the Fndc5 or a biologically active fragment thereof (e.g., irisin), but also its distribution in the examined tissue. Using the present invention, one of ordinary skill will readily perceive that any of a wide variety of histological methods (such as staining procedures) may be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier may be either soluble to some extent or insoluble for the purposes encompassed by the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Supports include, but are not limited to, polystyrene beads. Those skilled in the art will know many other suitable carriers for binding antibody or antigen, or will be able to ascertain the same by use of routine experimentation.

One means for labeling an antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, MD; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, FL, 1980; Ishikawa, et al., (eds.) Enzyme Immunoassay, KgakuShoin, Tokyo, 1981). The enzyme, which is bound to the antibody, will react with an appropriate substrate, such as a chromogenic substrate, in such a manner as to produce a chemical moiety that may be detected, for example, by spectrophotometric, fluorimetric or by visual means. Enzymes that may be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope may be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody may also be detectably labeled using fluorescence emitting metals such as ¹⁵²Eu, or others of the lanthanide series. These metals may be attached to the antibody using such metal chelating groups as diethylenetriaminepentacetic acid (DTPA) or ethylenediaminetetraacetic acid (EDTA).

The antibody also may be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody encompassed by the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

Additionally, also provided herein are methods of screening subjects by detecting duplication of the α-synuclein locus in the genome of cells within a subject to determine if the subject would benefit from a treatment method described herein. Duplication, triplication and of the α-synuclein locus can cause an α-synucleinopathy. Polymorphisms in the α-synuclein gene can increase or decrease one's rise of developing α-synucleinopathy, based on the expression of α-synuclein.

The present invention further provides methods for detecting single nucleotide polymorphisms in a gene encoding α-synuclein or duplication of the α-synuclein locus. Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism may be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide presents in the polymorphic site of the target molecule was complementary to that of the nucleotide derivative used in the reaction. This method has the advantage that it does not require the determination of large amounts of extraneous sequence data.

In another embodiment encompassed by the present invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is, in some embodiments, a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

Another method to detect the level of α-synuclein in the subject's cells includes an in vitro amplification technology, designated “real-time quaking-induced conversion (RT-QUIC),” for detection of a form of protein. This technique differs from other amplification techniques by “quaking” the sample. The “quaking” in the name of the technique refers to the fact that samples in the RT-QuIC assay are literally subjected to shaking. This action breaks apart aggregates of pathogenic protein that are then further incubated, and amplified to detectable levels. In some embodiments, patients are stratified and chosen for a method disclosed herein following RT-QUIC analysis of α-synuclein in the subject's cells. Further details regarding this technique include Bargar, C., et al. (2021). Streamlined α-synuclein RT-QuIC assay for various biospecimens in Parkinson's disease and dementia with Lewy bodies. Acta neuropathologica communications, 9(1), 62; Poggiolini, L., et al. (2021). Diagnostic value of cerebrospinal fluid α-synuclein seed quantification in synucleinopathies. Brain: a journal of neurology, awab431. Advance online publication; Zerr I. (202.1). RT-QuIC for detection of prodromal α-synucleinopathies. The Lancet. Neurology, 20(3), 165-166).

For determining the identity of the allelic variant of a polymorphic region located in the coding region of a gene encoding α-synuclein or a duplication of the α-synuclein locus, yet other methods than those described above may be used. For example, identification of an allelic variant that encodes a mutated protein may be performed by using an antibody specifically recognizing the mutant protein in, e.g., immunohistochemistry or immunoprecipitation.

### III. Methods of Treatment

The present invention provides for both prophylactic and therapeutic methods of preventing and/or treating a condition that would benefit from preventing or reducing degeneration of neurons, preventing or ameliorating at least one motor deficit and/or preventing or ameliorating at least one symptom of cognitive dysfunction or dementia, such as a subject afflicted with an α-synucleinopathy; or lowering the levels of α-synuclein, such as in a subject afflicted with an α-synucleinopathy or a cancer characterized by or caused by increased levels of α-synuclein, in a subject (e.g., a human) who is at risk of (or susceptible to) the condition, by administering to said subject an agent is selected from the group consisting of i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof, such that the condition is prevented or treated. In some embodiments, which includes both prophylactic and therapeutic methods, the agent is administered by in a pharmaceutically acceptable formulation.

Accordingly, one aspect of the present invention provides a method of reducing or lowering the levels of α-synuclein in cells (e.g., neuronal cells or cancer cells), the method comprising contacting the cells with an agent is selected from the group consisting of i) an Fndc5 polypeptide or a biologically active fragment thereof, ii) a nucleic acid that encodes an Fndc5 polypeptide or a biologically active fragment thereof, and iii) an enhancer of expression and/or activity of a Fndc5 polypeptide or biologically active fragment thereof, or nucleic acid encoding the Fndc5 polypeptide or biologically active fragment thereof, thereby lowering the levels of α-synuclein in the cells. In one embodiment, the biologically active Fndc5 fragment is irisin. This method may be performed in vivo, ex vivo, or in vitro. The cells or tissues may be in need of treatment if they are affected by a condition disclosed herein, such as an α-synucleinopathy or a cancer characterized by or caused by increased levels of α-synuclein.

In some embodiments, the biologically active Fndc5 fragment is irisin. In some embodiments, the agent is a polypeptide comprising an amino acid sequence having at least 70% identity to the amino acid sequence of SEQ ID NO: 2, wherein said fragment lacks the C-terminal domain sequence of said FNDC5 polypeptide, and wherein said polypeptide has one or more of the biological activities of said FNDC5 polypeptide. The agent may also be a polypeptide comprising an amino acid sequence having at least 70% identity to the amino acid sequence of residues 73-140 of the FNDC5 polypeptide of SEQ ID NO:2, wherein said polypeptide does not encode the C-terminal domain sequence of said FNDC5 polypeptide, and wherein said polypeptide has one or more of the biological activities of said FNDC5 polypeptide. Another form of the agent is a polypeptide fragment of the FNDC5 polypeptide of SEQ ID NO: 2, wherein the fragment consists of a sequence of amino acids in between residues 1 and 150 of SEQ ID NO: 2, and wherein the fragment has one or more of the biological activities of said FNDC5 polypeptide. In some embodiments, the agent is a polypeptide fragment of FNDC5 comprising an amino acid sequence having at least 70% identity to the amino acid sequence of a fragment of a FNDC5 polypeptide of SEQ ID NO: 4, 6 or 8, and wherein said fragment has one or more of the biological activities of said FNDC5 polypeptide.

The compositions disclosed herein may be administered over any period of time effective to achieve the desired therapeutic response for a particular patient, composition, and mode of administration, without being toxic to the patient. The period of time may be at least 1 day, at least 10 days, at least 20 days, at least 30, days, at least 60 days, at least three months, at least six months, at least a year, at least three years, at least five years, or at least ten years. The dose may be administered when needed, sporadically, or at regular intervals. For example, the dose may be administered monthly, weekly, biweekly, triweekly, once a day, or twice a day. In certain embodiments, a dose of the composition is administered at regular intervals over a period of time. In some embodiments, a dose of the composition is administered at least once a week. In some embodiments, a dose of the composition is administered at least twice a week. In certain embodiments, a dose of the composition is administered at least three times a week. In some embodiments, a dose of the composition is administered at least once a day. In some embodiments, a dose of the composition is administered at least twice a day. In some embodiments, doses of the composition are administered for at least 1 week, for at least 2 weeks, for at least 3 weeks, for at least 4 weeks, for at least 1 month, for at least 2 months, for at least 3 months, for at least 4 months, for at least 5 months, for at least 6 months, for at least 1 year, for at least two years, at least three years, or at least five years.

In some embodiments, the method further comprises contacting the cell and/or tissue, and/or administering to the subject with an additional agent that increases the expression or activity of Fndc5 or a biologically active fragment thereof. The biologically active fragment of Fndc5 may be irisin.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect encompassed by the present invention provides methods for tailoring a subject's prophylactic or therapeutic treatment with agents described according to that individual's drug response genotype. Pharmacogenomics allows a clinician or physician to target prophylactic or therapeutic treatments to patients who will most benefit from the treatment and to avoid treatment of patients who will experience toxic drug-related side effects.

### A. Prophylactic Methods

In one aspect, the present invention provides a method for preventing a condition in a subject that would benefit from lowering or reducing the levels of α-synuclein. As a non-limiting, representative example, a method of treating or preventing a α-synucleinopathy (e.g., Parkinsons' disease, Lewy body dementia, Alzheimer's disease, multiple system atrophy (MSA), or a neuroaxonal dystrophy) is provided involving administering to the subject with an Fndc5 polypeptide or a biologically active fragment thereof (e.g., irisin) or a nucleic acid that encodes an Fndc5 polypeptide or biologically active fragment thereof, and/or an enhancer of expression and/or activity of a Fndc5 polypeptide or biologically active fragment thereof or of nucleic acid encoding the Fndc5 polypeptide or biologically active fragment thereof. Subjects at risk for a an α-synucleinopathy or a cancer characterized by or caused by increased levels of α-synuclein may be identified by, for example, any or a combination of the diagnostic or prognostic assays described herein. The subjects may be at risk for a an α-synucleinopathy or a cancer characterized by or caused by increased levels of α-synuclein. Administration of a prophylactic agent can occur prior to the manifestation of symptoms characteristic of an α-synucleinopathy or a cancer characterized by or caused by increased levels of α-synuclein, such that the condition or symptom thereof, is prevented or, alternatively, delayed in its progression.

### B. Therapeutic Methods

In one aspect, the present invention provides a method for treating an α-synucleinopathy or a cancer characterized by or caused by increased levels of α-synuclein. As a non-limiting, representative example, a method of administering with an Fndc5 polypeptide or a biologically active fragment thereof (e.g., irisin) or a polynucleotide encoding said Fndc5 polypeptide or biologically active fragment thereof, and/or an enhancer of such a polypeptide/nucleic acid expression or activity is provided.

Accordingly, another aspect encompassed by the present invention pertains to methods of modulating expression or activity of Fndc5 or a biologically active fragment thereof (e.g., irisin) for therapeutic purposes and for use in treatment of a cancer characterized by or caused by increased levels of α-synuclein or an α-synucleinopathy, such as Parkinsons' disease, Lewy body dementia, multiple system atrophy (MSA), Alzheimer's disease, or a neuroaxonal dystrophy. In an exemplary embodiment, modulatory methods encompassed by the present invention involves reducing the level or amount of α-synuclein in the cells of a subject in need thereof, the method comprising administering to the subject with an Fndc5 polypeptide or a biologically active fragment thereof (e.g., irisin) or a polynucleotide encoding said Fndc5 polypeptide or biologically active fragment thereof, or an enhancer of such a polypeptide's or nucleic acid's expression or activity. In one embodiment, the agent simulates one or more activities of an Fndc5 polypeptide or a biologically active fragment thereof (e.g., irisin), or an enhancer of such a polypeptide's expression or activity. Examples of such stimulatory agents include small molecule agonists and mimetics, e.g., a peptidomimetic. These modulatory methods may be performed in vitro or ex vivo (e.g., by culturing the cell with the agent) or, alternatively, in vivo (e.g., by administering the agent to a subject). In one embodiment, the method involves administering an agent (e.g., an agent identified by a screening assay described herein), or combination of agents that modulate iris in expression or activity or are otherwise useful for treating or preventing an α-synucleinopathy or a cancer characterized by or caused by increased levels of α-synuclein.

Increasing expression or activity of Fndc5 or a biologically active fragment thereof (e.g., irisin) leads to treatment or prevention of the condition that would benefit from preventing or reducing degeneration of dopaminergic (DA) neurons, preventing or ameliorating at least one motor deficit and/or preventing or ameliorating at least one symptom of cognitive dysfunction or dementia, such as Parkinson's disease, Lewy body dementia, multiple system atrophy (MSA), Alzheimer's disease or a neuroaxonal dystrophy, therefore providing a method for treating, preventing, and/or assessing the condition of interest. A variety of techniques may be used to increase the expression, synthesis, or activity of Fndc5 or a biologically active fragment thereof (e.g., irisin) using an Fndc5 polypeptide or a biologically active fragment thereof (e.g., irisin) or a polynucleotide encoding said Fndc5 polypeptide or biologically active fragment thereof, or an enhancer of such a polypeptide's expression or activity.

For example, an Fndc5 polypeptide or a biologically active fragment thereof (e.g., irisin) or a polynucleotide encoding said Fndc5 polypeptide or biologically active fragment thereof, and/or an enhancer of such a polypeptide/polynucleotide expression or activity protein may be administered to a subject. Any of the techniques discussed below may be used for such administration. One of skill in the art will readily know how to determine the concentration of effective, non-toxic doses of the protein, utilizing techniques such as those described below.

Additionally, nucleic acid sequences, such as RNA sequences encoding such proteins may be directly administered to a subject, at a concentration sufficient to produce a level of an Fndc5 polypeptide or a biologically active fragment thereof (e.g., irisin), or an enhancer of such a polypeptide's expression or activity, such that expression or activity of Fndc5 or a biologically active fragment thereof (e.g., irisin) in cells is increased. Any of the techniques discussed below, which achieve intracellular administration of compounds, such as, for example, liposome administration, may be used for the administration of such nucleic acid molecules. RNA molecules may be produced, for example, by recombinant techniques such as those described herein. Other pharmaceutical compositions, medications, or therapeutics may be used in combination with the agents described herein. Further, subjects may be treated by gene replacement therapy. For example, one or more copies of a polynucleotide encoding an Fndc5 polypeptide or biologically active fragment thereof (e.g., irisin), or an enhancer of such a polypeptide's expression or activity, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of desired gene sequences into human cells. Furthermore, expression or activity of transcriptional activators which act upon Fndc5 may be increased to thereby increase expression or activity of Fndc5 or biologically active fragment thereof (e.g., irisin). Small molecules that enhance the expression or activity of Fndc5 or biologically active fragment thereof (e.g., irisin), either directly or indirectly, may also be used.

Cells, such as, autologous cells, containing Fndc5 expressing gene sequences may then be introduced or reintroduced into the subject. Such cell replacement techniques may be well-suited for use in treating or preventing a disease, for example, when the gene product is a secreted, extracellular gene product.

### IV. Pharmaceutical Compositions

In some embodiments, methods encompassed by the present invention involve the use of an agent that increases expression or activity of Fndc5 or biologically active fragment thereof (e.g., irisin), such as an enhancer of such a polypeptide's expression or activity, either alone or in combination with other agents useful for treating or preventing an α-synucleinopathy or a cancer characterized by or caused by increased levels of α-synuclein.

Such agents that increase expression or activity Fndc5 or biologically active fragment thereof (e.g., irisin) may be formulated as pharmaceutical compositions. They may be administered in a therapeutically effective amount to a subject using pharmaceutical compositions suitable for such administration. Such compositions typically comprise the agent (e.g., nucleic acid molecule or protein) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary active compounds may also be incorporated into the compositions.

The term “effective amount” of an agent that induces expression and/or activity of Fndc5 or biologically active fragment thereof (e.g., irisin) is that amount necessary or sufficient to increase expression and/or activity of Fndc5 or biologically active fragment thereof (e.g., irisin) in the appropriate context, such as cells in vitro or ex vivo, a subject, a population of subjects, and the like. An effective amount can vary depending on such factors as the type of therapeutic agent(s) employed, the size of the subject, or the severity of the disorder.

The term “therapeutically effective amount” as used herein means that amount of an agent that increases the expression or activity of Fndc5 or biologically active fragment thereof (e.g., irisin), or composition comprising an agent that increases such expression or activity, which is effective for producing some desired therapeutic effect, e.g., expression or activity of Fndc5 or biologically active fragment thereof (e.g., irisin) in subjects, at a reasonable benefit/risk ratio.

A pharmaceutical composition used in therapeutic methods encompassed by the present invention may be formulated to be compatible with its intended route of administration. Administration may be systemic or local. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH may be adjusted with acids or bases, such as hydrochloric acid or sodium hydroxide. The parenteral preparation may be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, polyalcohols such as mannitol, sorbitol, and sodium chloride are included in the composition. Prolonged absorption of the injectable compositions may be brought about by including in the composition an agent which delays absorption, for example, aluminum monostearate and gelatin.

Sterile injectable solutions may be prepared by incorporating Fndc5 or biologically active fragment thereof (e.g., irisin), or an enhancer of such a polypeptide's expression or activity, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, some methods of preparation are vacuum drying and freeze-drying which yields a powder of the active ingredient plus any additional desired ingredient from a previously sterile-filtered solution thereof.

Oral compositions generally include an inert diluent or an edible carrier. They may be enclosed in gelatin capsules or compressed into tablets. For the purpose of oral therapeutic administration, the active compound may be incorporated with excipients and used in the form of tablets, troches, or capsules. Oral compositions may also be prepared using a fluid carrier for use as a mouthwash, wherein the compound in the fluid carrier is applied orally and swished and expectorated or swallowed. Pharmaceutically compatible binding agents, and/or adjuvant materials may be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration may also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art.

The agents that modulate Fndc5 or irisin expression or activity may also be prepared in the form of suppositories (e.g., with conventional suppository bases such as cocoa butter and other glycerides) or retention enemas for rectal delivery.

In one embodiment, the agents that modulate Fndc5 or irisin expression or activity are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. Methods for preparation of such formulations will be apparent to those skilled in the art. The materials may also be obtained commercially from Alza Corporation and Nova Pharmaceuticals, Inc. Liposomal suspensions (including liposomes targeted to infected cells with monoclonal antibodies to viral antigens) may also be used as pharmaceutically acceptable carriers. These may be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. In some embodiments, the specification for the dosage unit forms encompassed by the present invention are dictated by and directly dependent on the unique characteristics of the agent that modulates Fndc5 activity and the particular therapeutic effect to be achieved, and the limitations inherent in the art of compounding such an agent for the treatment of subjects.

Toxicity and therapeutic efficacy of such agents may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and may be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies may be used in formulating a range of dosage for use in humans. In certain embodiments, the dosage of such Fndc5 or irisin modulating agents lies within a range of circulating concentrations that include the ED50 with little or no toxicity. The dosage may vary within this range depending upon the dosage form employed and the route of administration utilized. For any agent used in therapeutic methods encompassed by the present invention, the therapeutically effective dose may be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, from about 0.01 to 25 mg/kg body weight, from about 0.1 to 20 mg/kg body weight, or from about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or a series of treatments.

In a one example, a subject is treated with a polypeptide in the range of between about 0.1 to 20 mg/kg body weight, one time per week for between about 1 to 10 weeks, between 2 to 8 weeks, between about 3 to 7 weeks, or for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents that modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect that the practitioner desires the small molecule to have upon a nucleic acid or polypeptide encompassed by the present invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of Fndc5 or biologically active fragment thereof (e.g., irisin), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated, e.g., the intended use of the agonist or antagonize.

Further, agents described herein may be conjugated to additional therapeutic moieties of interest, such as a growth factor, intracellular targeting domain, and the like, that are well-known in the art. Conjugates encompassed by the present invention may be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Nucleic acid molecules encompassed by the methods of the present invention may be inserted into vectors and used as gene therapy vectors. Gene therapy vectors may be delivered to a subject by, for example, intravenous injection, local administration (see U.S. Pat. No. 5,328,470) or by stereotactic injection (see, e.g., Chen et al. (1994) Proc. Natl. Acad. Sci. U.S.A. 91:3054-3057). A pharmaceutical preparation of the gene therapy vector may include the gene therapy vector in an acceptable diluent, or may comprise a slow release matrix in which the gene delivery vehicle is imbedded. Alternatively, where the complete gene delivery vector may be produced intact from recombinant cells, e.g., retroviral vectors, the pharmaceutical preparation may include one or more cells that produce the gene delivery system.

As defined herein, a therapeutically effective amount of vector particle (i.e., an effective dosage) ranges from at least 1×10² GC/kg particles, at least 1×10³ GC/kg particles, at least 1×104 GC/kg particles, at least 1×10⁵ GC/kg particles, at least 1×10⁶ GC/kg particles, at least 1×10⁷ GC/kg particles, at least 1×10⁸ GC/kg particles, at least 1×10⁹ GC/kg particles, at least 1×10¹⁰ GC/kg particles, at least 1×10¹¹ GC/kg particles, at least 1×10¹² GC/kg particles, at least 1×10¹³ GC/kg particles, at least 1×10¹⁴ GC/kg particles, at least 1×10¹⁵ GC/kg particles, at least 1×10¹⁶ GC/kg particles, at least 1×10¹⁷ GC/kg particles, at least 1×10¹⁸ GC/kg particles, at least 1×10¹⁹ GC/kg particles, at least 1×10²⁰ GC/kg particles, at least 1×10²¹ GC/kg particles, at least 1×10²² GC/kg particles, at least 1×10²³ GC/kg particles, at least 1×10²⁴ GC/kg particles, at least 1×10²⁵ GC/kg particles, at least 1×10²⁶ GC/kg particles, at least 1×10²⁷ GC/kg particles, at least 1×10²⁸ GC/kg particles, at least 1×10²⁶ GC/kg particles, at least 1×10²⁷ GC/kg particles, at least 1×10²⁸ GC/kg particles, at least 1×10²⁹ GC/kg particles, or at least 1×10³⁰ GC/kg particles. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a nucleic acid vector can include a single treatment or a series of treatments.

Any means for the introduction of a polynucleotide into mammals, human or non-human, or cells thereof may be adapted to the practice of the present for the delivery of various constructs encompassed by the present invention into the intended recipient. In one embodiment encompassed by the present invention, the DNA constructs are delivered to cells by transfection, i.e., by delivery of “naked” DNA or in a complex with a colloidal dispersion system. A colloidal system includes macromolecule complexes, nanocapsules, microspheres, beads, and lipid-based systems including oil-in-water emulsions, micelles, mixed micelles, and liposomes. In certain embodiments, the colloidal system of this invention is a lipid-complexed or liposome-formulated DNA. In the former approach, prior to formulation of DNA, e.g., with lipid, a plasmid containing a transgene bearing the desired DNA constructs may first be experimentally optimized for expression (e.g., inclusion of an intron in the 5′ untranslated region and elimination of unnecessary sequences (Felgner et al. (1995) Ann. NY Acad. Sci., 126-139). Formulation of DNA, e.g. with various lipid or liposome materials, may then be effected using known methods and materials and delivered to the recipient mammal. See, e.g., Canonico et al. (1994) Am. J. Respir. Cell. Mol. Biol., 10:24-29; Tsan et al. (1994) Am. J. Physiol., 268; Alton et al. (1993) Nat. Genet., 5:135-142; and U.S. Pat. No. 5,679,647 by Carson et al.

The targeting of liposomes may be classified based on anatomical and mechanistic factors. Anatomical classification is based on the level of selectivity, for example, organ-specific, cell-specific, and organelle-specific. Mechanistic targeting may be distinguished based upon whether it is passive or active. Passive targeting utilizes the natural tendency of liposomes to distribute to cells of the reticulo-endothelial system (RES) in organs, which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization.

The surface of the targeted delivery system may be modified in a variety of ways. In the case of a liposomal targeted delivery system, lipid groups may be incorporated into the lipid bilayer of the liposome in order to maintain the targeting ligand in stable association with the liposomal bilayer. Various linking groups may be used for joining the lipid chains to the targeting ligand. Naked DNA or DNA associated with a delivery vehicle, e.g., liposomes, may be administered to several sites in a subject (see below).

Nucleic acids may be delivered in any desired vector. These include viral or non-viral vectors, including adenovirus vectors, adeno-associated virus vectors, retrovirus vectors, lentivirus vectors, and plasmid vectors. Exemplary types of viruses include HSV (herpes simplex virus), AAV (adeno-associated virus), HIV (human immunodeficiency virus), BIV (bovine immunodeficiency virus), and MLV (murine leukemia virus). Nucleic acids may be administered in any desired format that provides sufficiently efficient delivery levels, including in virus particles, in liposomes, in nanoparticles, and complexed to polymers.

Nucleic acids encoding a protein or nucleic acids of interest may be in a plasmid or viral vector, or other vector as is known in the art. Such vectors are well-known and any may be selected for a particular application. In one embodiment encompassed by the present invention, the gene delivery vehicle comprises a promoter and a demethylase coding sequence. In some embodiments, promoters are tissue-specific promoters and promoters, which are activated by cellular proliferation, such as the thymidine kinase and thymidylate synthase promoters. Other promoters include promoters, which are activated by infection with a virus, such as the α- and β-interferon promoters, and promoters, which are activated by a hormone, such as estrogen. Other promoters that may be used include the Moloney virus LTR, the CMV promoter, and the mouse albumin promoter. A promoter may be constitutive or inducible.

In another embodiment, naked polynucleotide molecules are used as gene delivery vehicles, as described in WO 90/11092 and U.S. Pat. No. 5,580,859. Such gene delivery vehicles may be either growth factor DNA or RNA and, in certain embodiments, are linked to killed adenovirus. Curiel et al., Hum. Gene. Ther. 3:147-154, 1992. Other vehicles that can optionally be used include DNA-ligand (Wu et al., J. Biol. Chem. 264:16985-16987, 1989), lipid-DNA combinations (Felgner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

A gene delivery vehicle can optionally comprise viral sequences such as a viral origin of replication or packaging signal. These viral sequences may be selected from viruses such as astrovirus, coronavirus, orthomyxovirus, papovavirus, paramyxovirus, parvovirus, picornavirus, poxvirus, retrovirus, togavirus or adenovirus. In an embodiment, the growth factor gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles may be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Other viral vector systems that may be used to deliver a polynucleotide encompassed by the present invention have been derived from herpes virus, e.g., Herpes Simplex Virus (U.S. Pat. No. 5,631,236 by Woo et al., issued May 20, 1997 and WO 00/08191 by Neurovex), vaccinia virus (Ridgeway (1988) Ridgeway, “Mammalian expression vectors,” In: Rodriguez R L, Denhardt D T, ed. Vectors: A survey of molecular cloning vectors and their uses. Stoneham: Butterworth, Baichwal and Sugden (1986) “Vectors for gene transfer derived from animal DNA viruses: Transient and stable expression of transferred genes,” In: Kucherlapati R, ed. Gene transfer. New York: Plenum Press; Coupar et al. (1988) Gene, 68:1-10), and several RNA viruses. In Viruses include, but are not limited to, an alphavirus, a poxivirus, an arena virus, a vaccinia virus, a polio virus, and the like. They offer several attractive features for various mammalian cells (Friedmann (1989) Science, 244:1275-1281; Ridgeway, 1988, supra; Baichwal and Sugden, 1986, supra; Coupar et al., 1988; Horwich et al., (1990) J. Virol., 64:642-650).

In other embodiments, target DNA in the genome may be manipulated using well-known methods in the art. For example, the target DNA in the genome may be manipulated by deletion, insertion, and/or mutation are retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis. In some embodiments, genome editing may be used to modulate the copy number or genetic sequence of a protein of interest, such as constitutive or induced knockout or mutation of a protein of interest, such as a Fndc5 or a biologically active fragment thereof (e.g., irisin) encompassed by the present invention. For example, the CRISPR-Cas system may be used for precise editing of genomic nucleic acids (e.g., for creating non-functional or null mutations). In such embodiments, the CRISPR guide RNA and/or the Cas enzyme may be expressed. For example, a vector containing only the guide RNA may be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

In other embodiments, recombinant Fndc5 or a biologically active fragment thereof (e.g., irisin), may be administered to subjects. In some embodiments, fusion proteins may be constructed that have enhanced biological properties (e.g., Fc fusion proteins discussed above) and administered. In addition, Fndc5 polypeptide or a biologically active fragment thereof (e.g., irisin), may be modified according to well-known pharmacological methods in the art (e.g., pegylation, glycosylation, oligomerization, etc.) in order to further enhance desirable biological activities, such as increased bioavailability and decreased proteolytic degradation.

### V. Predictive Medicine

The present invention also pertains to the field of predictive medicine in which diagnostic assays, prognostic assays, and monitoring of clinical trials are used for prognostic (predictive) purposes to thereby treat an individual prophylactically. Accordingly, one aspect of the present invention relates to diagnostic assays for determining the levels of protein and/or nucleic acid expression or activity of, in the context of a biological sample (e.g., blood, serum, fluid, cells, or tissue, e.g., cancer cells or neuronal cells or tissue) to thereby determine whether an individual is afflicted with a condition that would benefit from reducing or lowing the level or amount of α-synuclein, or has a risk of developing the condition. The present invention also provides for prognostic (or predictive) assays for determining whether an individual is at risk of developing the condition.

One particular embodiment includes a method for assessing whether a subject is afflicted with an α-synucleinopathy or a cancer characterized by or caused by an increase in α-synuclein or is at risk of developing an α-synucleinopathy or a cancer characterized by or caused by an increase in α-synuclein comprising detecting the expression or activity of Fndc5 or a biologically active fragment thereof (e.g., irisin) in cell (e.g., a cancer cell or a neuronal cell), such as from a sample from a subject, wherein a decrease in the expression or activity thereof indicates the presence of a α-synucleinopathy or a cancer characterized by or caused by an increase in α-synuclein or the risk of developing a α-synucleinopathy or a cancer characterized by or caused by an increase in α-synuclein in the subject. Subject samples tested may comprise, for example, cancer cells or neuronal cells.

Another aspect encompassed by the present invention pertains to monitoring the influence of agents that increase expression or activity of Fndc5 or a biologically active fragment thereof (e.g., irisin) in clinical trials.

### A. Prognostic and Diagnostic Assays

To determine whether a subject is afflicted with a condition disclosed herein, or has a risk of developing such a condition, a biological sample may be obtained from a subject and the biological sample may be contacted with a compound or an agent capable of detecting an Fndc5 polypeptide or a biologically active fragment thereof (e.g., irisin) or a polynucleotide (e.g., mRNA or genomic DNA) encoding said Fndc5 polypeptide or biologically active fragment thereof, in the biological sample. An agent for detecting the mRNA or genomic DNA may comprise a labeled nucleic acid probe capable of hybridizing to the mRNA or genomic DNA. The nucleic acid probe may be, for example, a sequence that is complementary to an Fndc5 or irisin nucleic acid set forth in Table 1, or a portion thereof, such as an oligonucleotide of at least 15, 20, 25, 30, 25, 40, 45, 50, 100, 250 or 500 nucleotides in length and sufficient to specifically hybridize under stringent conditions to the desired mRNA or genomic DNA. Other suitable probes for use in diagnostic assays encompassed by the present invention are described herein.

The term “biological sample” is intended to include tissues, cells, and biological fluids isolated from a subject, as well as tissues, cells, and fluids present within a subject. That is, a detection method encompassed by the present invention may be used to detect mRNA, protein, or genomic DNA of Fndc5 or a biologically active fragment thereof (e.g., irisin) in a biological sample in vitro, as well as in vivo. For example, in vitro techniques for detection of mRNA include Northern hybridizations and in situ hybridizations. In vitro techniques for detection of protein include enzyme linked immunosorbent assays (ELISAs), Western blots, immunoprecipitations and immunofluorescence. In vitro techniques for detection of genomic DNA include Southern hybridizations. Furthermore, in vivo techniques for detection of protein include introducing into a subject a labeled antibody against the desired protein to be detected. For example, the antibody may be labeled with a radioactive marker whose presence and location in a subject may be detected by standard imaging techniques.

In another embodiment, the methods further involve obtaining a control biological sample from a control subject, contacting the control sample with a compound or agent capable of detecting protein, mRNA, or genomic DNA, such that the presence of the desired protein, mRNA or genomic DNA is detected in the biological sample, and comparing the presence of the protein, mRNA or genomic DNA in the control sample with the presence of the protein, mRNA or genomic DNA in the test sample.

Analysis of one or more polymorphic regions of nucleic acids of Fndc5 or a biologically active fragment thereof (e.g., irisin) in a subject may be useful for predicting whether a subject has or is likely to develop a condition that would benefit from a decrease in the level or amount of α-synuclein in cells of the subject. In some embodiments, methods encompassed by the present invention may be characterized as comprising detecting, in a sample of cells from the subject, the presence or absence of a specific allelic variant of one or more polymorphic regions of the gene, such as a premature truncation that does not encode a biologically active protein or a mutation in the stop codon. The allelic differences may be: (i) a difference in the identity of at least one nucleotide or (ii) a difference in the number of nucleotides, which difference may be a single nucleotide or several nucleotides. The present invention also provides methods for detecting differences in a gene encoding Fndc5 or a biologically active fragment thereof (e.g., irisin), such as chromosomal rearrangements, e.g., chromosomal dislocation. The present invention may also be used in prenatal diagnostics.

A detection method may be allele-specific hybridization using probes overlapping the polymorphic site and having about 5, 10, 20, 25, or 30 nucleotides around the polymorphic region. In one embodiment encompassed by the present invention, several probes capable of hybridizing specifically to allelic variants are attached to a solid phase support, e.g., a “chip.” Oligonucleotides may be bound to a solid support by a variety of processes, including lithography. For example, a chip may hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes may be identified in a simple hybridization experiment. For example, the identity of the allelic variant of the nucleotide polymorphism in the 5′ upstream regulatory element may be determined in a single hybridization experiment.

In other detection methods, it is necessary to first amplify at least a portion of nucleic acid prior to identifying the allelic variant. Amplification may be performed, e.g., by PCR and/or LCR (see Wu and Wallace, (1989) Genomics 4:560), according to methods known in the art. In one embodiment, genomic DNA of a cell is exposed to two PCR primers and amplification for a number of cycles sufficient to produce the required amount of amplified DNA. In some embodiments, the primers are located between 150 and 350 base pairs apart.

Alternative amplification methods include: self-sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio Technology 6:1197), and self-sustained sequence replication (Guatelli et al., (1989) Proc. Nat. Acad. Sci. 87:1874), and nucleic acid based sequence amplification (NABSA), or any other nucleic acid amplification method, followed by the detection of the amplified molecules using techniques well-known to those of skill in the art. These detection schemes are especially useful for the detection of nucleic acid molecules if such molecules are present in very low numbers.

In one embodiment, any of a variety of sequencing reactions known in the art may be used to directly sequence at least a portion of an Fndc5- or irisin-encoding gene, or portion thereof, and detect allelic variants, e.g., mutations, by comparing the sequence of the sample sequence with the corresponding reference (control) sequence. Exemplary sequencing reactions include those based on techniques developed by Maxam and Gilbert (Proc. Natl Acad Sci USA (1977) 74:560) or Sanger (Sanger et al. (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Köster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, may be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of an Fndc5- or irisin-encoding gene in DNA from a subject may be shown by restriction enzyme analysis. For example, a specific nucleotide polymorphism can result in a nucleotide sequence comprising a restriction site that is absent from the nucleotide sequence of another allelic variant.

In a further embodiment, protection from cleavage agents (such as a nuclease, hydroxylamine or osmium tetroxide and with piperidine) may be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of an Fndc5 allelic variant with a sample nucleic acid, e.g., RNA or DNA, obtained from a tissue sample. The double-stranded duplexes are treated with an agent that cleaves single-stranded regions of the duplex such as duplexes formed based on base pair mismatches between the control and sample strands. For instance, RNA/DNA duplexes may be treated with RNase and DNA/DNA hybrids treated with Si nuclease to enzymatically digest the mismatched regions. In other embodiments, either DNA/DNA or RNA/DNA duplexes may be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al. (1988) Proc. Natl Acad Sci USA 85:4397; Saleeba et al. (1992) Methods Enzymol. 217:286-295. In one embodiment, the control or sample nucleic acid is labeled for detection.

In another embodiment, an allelic variant may be identified by denaturing high-performance liquid chromatography (DHPLC) (Oefner and Underhill (1995) Am. J. Human Gen. 57:Suppl. A266). DIPLC uses reverse-phase ion-pairing chromatography to detect the heteroduplexes that are generated during amplification of PCR fragments from individuals who are heterozygous at a particular nucleotide locus within that fragment (Oefner and Underhill (1995) Am. J. Human Gen. 57:Suppl. A266). In general, PCR products are produced using PCR primers flanking the DNA of interest. DHPLC analysis is carried out and the resulting chromatograms are analyzed to identify base pair alterations or deletions based on specific chromatographic profiles (see O'Donovan et al. (1998) Genomics 52:44-49).

In other embodiments, alterations in electrophoretic mobility is used to identify the type of desired allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766; see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions that permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of Fndc5- or irisin-encoding genes. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology that depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition, it may be desirable to introduce a novel restriction site in the region of the mutation to create cleavage-based detection (Gasparini et al. (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., (1988) Science 241:1077-1080. The OLA protocol uses two oligonucleotides that are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., (1990) Proc. Natl. Acad. Sci. (U.S.A.) 87:8923-8927. In this method, PCR is used to achieve the exponential amplification of target DNA, which is then detected using OLA.

Several techniques based on this OLA method have been developed and may be used to detect specific allelic variants of a polymorphic region of an Fndc5 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction may be detected by using hapten specific antibodies that are labeled with different enzyme reporters, alkaline phosphatase or horseradish peroxidase. This system permits the detection of the two alleles using a high throughput format that leads to the production of two different colors.

The present invention further provides methods for detecting single nucleotide polymorphisms in agene encoding Fndc5 or a biologically active fragment thereof (e.g., irisin). Because single nucleotide polymorphisms constitute sites of variation flanked by regions of invariant sequence, their analysis requires no more than the determination of the identity of the single nucleotide present at the site of variation and it is unnecessary to determine a complete gene sequence for each subject. Several methods have been developed to facilitate the analysis of such single nucleotide polymorphisms.

In one embodiment, the single base polymorphism may be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide presents in the polymorphic site of the target molecule was complementary to that of the nucleotide derivative used in the reaction. This method has the advantage that it does not require the determination of large amounts of extraneous sequence data.

In another embodiment encompassed by the present invention, a solution-based method is used for determining the identity of the nucleotide of a polymorphic site (Cohen, D. et al. (French Patent 2,650,840; PCT Application No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Application No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is, in some embodiments, a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For determining the identity of the allelic variant of a polymorphic region located in the coding region of a gene encoding Fndc5 or a biologically active fragment thereof (e.g., irisin), yet other methods than those described above may be used. For example, identification of an allelic variant that encodes a mutated protein may be performed by using an antibody specifically recognizing the mutant protein in, e.g., immunohistochemistry or immunoprecipitation. Antibodies to wild-type Fndc5 or a biologically active fragment thereof (e.g., irisin), or mutated forms of such proteins may be prepared according to methods known in the art.

Antibodies directed against reference or mutant Fndc5 or a biologically active fragment thereof (e.g., irisin) may also be used in disease diagnostics and prognostics. Such antibodies are well-known in the art (see, for example, antibody LS-C166197 from Lifespan Biosciences, antibody AG-25B-0027 from Adipogen, antibody HPA051290 from Atlas Antibodies, antibody PAN576Hu02 from UscnLifescienes, antibody OACD03594 from Aviva Systems Biology, antibody NBP2-14024 from Novus Biologicals, and the like). In addition, such diagnostic methods, may be used to detect abnormalities in the level of such polypeptide expression, or abnormalities in the structure and/or tissue, cellular, or subcellular location of such polypeptides. Structural differences may include, for example, differences in the size, electronegativity, or antigenicity of the mutant polypeptide relative to the normal polypeptide. Protein from the tissue or cell type to be analyzed may easily be detected or isolated using techniques that are well-known to one of skill in the art, including but not limited to Western blot analysis. For a detailed explanation of methods for carrying out Western blot analysis, see Sambrook et al, 1989, supra, at Chapter 18. The protein detection and isolation methods employed herein may also be such as those described in Harlow and Lane, for example (Harlow, E. and Lane, D., 1988, “Antibodies: A Laboratory Manual”, Cold Spring Harbor Laboratory Press, Cold Spring Harbor, New York), which is incorporated herein by reference in its entirety.

This may be accomplished, for example, by immunofluorescence techniques employing a fluorescently labeled antibody (see below) coupled with light microscopic, flow cytometric, or fluorimetric detection. The antibodies (or fragments thereof) useful according to the present invention may, additionally, be employed histologically, as in immunofluorescence or immunoelectron microscopy, for in situ detection of Fndc5 or a biologically active fragment thereof (e.g., irisin). In situ detection may be accomplished by removing a histological specimen from a subject, and applying thereto a labeled antibody of the present invention. The antibody (or fragment) is may be applied by overlaying the labeled antibody (or fragment) onto a biological sample. Through the use of such a procedure, it is possible to determine not only the presence of the Fndc5 or a biologically active fragment thereof (e.g., irisin), but also its distribution in the examined tissue. Using the present invention, one of ordinary skill will readily perceive that any of a wide variety of histological methods (such as staining procedures) may be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier may be either soluble to some extent or insoluble for the purposes encompassed by the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Supports include, but are not limited to, polystyrene beads. Those skilled in the art will know many other suitable carriers for binding antibody or antigen, or will be able to ascertain the same by use of routine experimentation.

One means for labeling an antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, MD; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, FL, 1980; Ishikawa, et al., (eds.) Enzyme Immunoassay, KgakuShoin, Tokyo, 1981). The enzyme, which is bound to the antibody, will react with an appropriate substrate, such as a chromogenic substrate, in such a manner as to produce a chemical moiety that may be detected, for example, by spectrophotometric, fluorimetric or by visual means. Enzymes that may be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection may be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope may be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine. The antibody may also be detectably labeled using fluorescence emitting metals such as ¹⁵²Eu, or others of the lanthanide series. These metals may be attached to the antibody using such metal chelating groups as diethylenetriaminepentacetic acid (DTPA) or ethylenediaminetetraacetic acid (EDTA).

The antibody also may be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody encompassed by the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

If a polymorphic region is located in an exon, either in a coding or non-coding portion of the gene, the identity of the allelic variant may be determined by determining the molecular structure of the mRNA, pre-mRNA, or cDNA. The molecular structure may be determined using any of the above described methods for determining the molecular structure of the genomic DNA.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific allelic variant of interest. Sample nucleic acid to be analyzed by any of the above-described diagnostic and prognostic methods may be obtained from any cell type or tissue of a subject. For example, a subject's bodily fluid (e.g. blood) may be obtained by known techniques (e.g., venipuncture). Alternatively, nucleic acid tests may be performed on dry samples (e.g., hair or skin). Fetal nucleic acid samples may be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of subject tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods that focus primarily on the detection of one nucleic acid sequence, profiles may also be assessed in such detection schemes. Fingerprint profiles may be generated, for example, by utilizing a differential display procedure, Northern analysis and/or RT-PCR.

### B. Monitoring of Effects During Clinical Trials

The present invention further provides methods for determining the effectiveness of an Fndc5 or a biologically active fragment thereof (e.g., irisin), or enhancer of expression or activity thereof, in treating or preventing a condition that would benefit from reducing or lowering the levels or amount of α-synuclein, and the like, or assessing risk of developing such a condition (e.g., a condition disclosed herein). For example, the effectiveness of such an enhancer of expression or activity of Fndc5 or a biologically active fragment thereof (e.g., irisin) may be monitored in clinical trials of subjects. In such clinical trials, the expression or activity of Fndc5 or a biologically active fragment thereof (e.g., irisin) that have been implicated in, for example, an expression pathway of Fndc5 or a biologically active fragment thereof (e.g., irisin) may be used as a “read out” or marker of the phenotype of a particular cell.

For example, and not by way of limitation, genes, including Fndc5 or a biologically active fragment thereof (e.g., irisin), that are modulated in cells by treatment with an agent that increases expression or activity Fndc5 or a biologically active fragment thereof (e.g., irisin) may be identified. Thus, to study the effect of agents that increases expression or activity of Fndc5 or a biologically active fragment thereof (e.g., irisin) in subjects suffering from or at risk of developing the condition, or agents to be used as a prophylactic, for example, a clinical trial, cells may be isolated and RNA prepared and analyzed for the levels of expression or activity of Fndc5 or a biologically active fragment thereof (e.g., irisin), and other genes implicated in the pathway of Fndc5 or a biologically active fragment thereof (e.g., irisin). The levels of gene expression (e.g., a gene expression pattern) may be quantified by Northern blot analysis or RT-PCR, as described herein, or alternatively by measuring the amount of protein produced, by one of the methods described herein, or by measuring the levels of activity of Fndc5 or a biologically active fragment thereof (e.g., irisin). In this way, the gene expression pattern may serve as a marker, indicative of the physiological response of the cells to the agent that increases expression or activity of Fndc5 or a biologically active fragment thereof (e.g., irisin). This response state may be determined before, and at various points during treatment of the individual with the agent that increases expression or activity of Fndc5 or a biologically active fragment thereof (e.g., irisin).

In one embodiment, the present invention provides a method for monitoring the effectiveness of treatment of a subject with an agent that increases expression or activity of Fndc5 or a biologically active fragment thereof (e.g., irisin) (e.g., an agonist, antagonist, peptidomimetic, protein, peptide, nucleic acid, siRNA, or small molecule identified by the screening assays described herein) including the steps of (i) obtaining a pre-administration sample from a subject prior to administration of the agent, such as a sample comprising cancer cells or neuronal cells; (ii) detecting the level of expression of an Fndc5 or irisin protein, mRNA, or genomic DNA in the pre-administration sample; (iii) obtaining one or more post-administration samples from the subject; (iv) detecting the level of expression or activity of Fndc5 or a biologically active fragment thereof (e.g., irisin), such as by analyzing protein, mRNA, or genomic DNA, in the post-administration samples; (v) comparing the level of expression or activity of the Fndc5 or a biologically active fragment thereof (e.g., irisin) in the pre-administration sample with the Fndc5 or a biologically active fragment thereof (e.g., irisin) in the post administration sample or samples; and (vi) altering the administration of the agent to the subject accordingly. For example, increased administration of the agent may be desirable to increase the expression or activity of Fndc5 or a biologically active fragment thereof (e.g., irisin) to higher levels than detected, i.e., to increase the effectiveness of the agent. According to such an embodiment, expression or activity of Fndc5 or a biologically active fragment thereof (e.g., irisin) may be used as an indicator of the effectiveness of an agent, even in the absence of an observable phenotypic response.

### VI. Isolated Nucleic Acids, Polypeptides, Antibodies, Vectors, and Host Cells Useful for Methods Described Herein

Nucleic acids, polypeptides, vectors, and host cells related to Fndc5 or a biologically active fragment thereof (e.g., irisin), are useful for carrying out the methods described herein.

Isolated nucleic acid molecules that encode Fndc5 or a biologically active fragment thereof (e.g., irisin), are well-known in the art. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule may be single-stranded or double-stranded. An “isolated” nucleic acid molecule is one that is separated from other nucleic acid molecules that are present in the natural source of the nucleic acid. In some embodiments, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated Fndc5 nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived (i.e., a brown adipocyte). Moreover, an “isolated” nucleic acid molecule, such as a cDNA molecule, may be substantially free of other cellular material, or culture medium when produced by recombinant techniques, or chemical precursors or other chemicals when chemically synthesized.

A nucleic acid molecule encompassed by the present invention, e.g., a nucleic acid molecule having the nucleotide sequence of SEQ ID NO: 1, 3, 5, 7, 9, 11, 13 and 15 or a nucleotide sequence that is at least about 50%, at least about 60%, at least about 70%, at least about 80%, at least about 90%, or at least about 95% or more (e.g., about 98%) homologous to the nucleotide sequence shown in SEQ ID NOs: 1, 3, 5, 7, 9, 11, 13 and 15 or a portion thereof (i.e., 100, 200, 300, 400, 450, 500, or more nucleotides), may be isolated using standard molecular biology techniques and the sequence information provided herein. For example, a human Fndc5 cDNA may be isolated from a human cell line using all or portion of SEQ ID NOs: 1, 3, 5, 7, 9, 11, 13 or 15, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989). Moreover, a nucleic acid molecule encompassing all or a portion of SEQ ID NOs: 1, 3, 5, 7, 9, 11, 13 or 15 or a nucleotide sequence that is at least about 50%, at least about 60%, at least about 70%, at least about 80%, at least about 90%, or at least about 95% or more homologous to the nucleotide sequence shown in SEQ ID NOs: 1, 3, 5, 7, 9, 11, 13 or 15, or fragment thereof, may be isolated by the polymerase chain reaction using oligonucleotide primers designed based upon the sequence of SEQ ID NOs: 1, 3, 5, 7, 9, 11, 13 or 15, or fragment thereof, or the homologous nucleotide sequence. For example, mRNA may be isolated from cells disclosed herein (i.e., by the guanidinium-thiocyanate extraction procedure of Chirgwin et al. (1979) Biochemistry 18: 5294-5299) and cDNA may be prepared using reverse transcriptase (i.e., Moloney MLV reverse transcriptase, available from Gibco/BRL, Bethesda, MD; or AMV reverse transcriptase, available from Seikagaku America, Inc., St. Petersburg, FL). Synthetic oligonucleotide primers for PCR amplification may be designed based upon the nucleotide sequence shown in SEQ ID NOs: 1, 3, 5, 7, 9, 11, 13 or 15, or fragment thereof, or to the homologous nucleotide sequence. A nucleic acid encompassed by the present invention may be amplified using cDNA or, alternatively, genomic DNA, as a template and appropriate oligonucleotide primers according to standard PCR amplification techniques. The nucleic acid so amplified may be cloned into an appropriate vector and characterized by DNA sequence analysis. Furthermore, oligonucleotides corresponding to an Fndc5 nucleotide sequence may be prepared by standard synthetic techniques, i.e., using an automated DNA synthesizer.

Probes based on the Fndc5 nucleotide sequences may be used to detect transcripts or genomic sequences encoding the same or homologous proteins. In certain embodiments, the probe further comprises a label group attached thereto, i.e., the label group may be a radioisotope, a fluorescent compound, an enzyme, or an enzyme co-factor. Such probes may be used as a part of a diagnostic test kit for identifying cells or tissue that express an Fndc5 protein, such as by measuring a level of an Fndc5-encoding nucleic acid in a sample of cells from a subject, i.e., detecting Fndc5 mRNA levels.

Nucleic acid molecules encoding other Fndc5 members and thus have a nucleotide sequence that differs from the Fndc5 sequences of SEQ ID NOs: 1, 3, 5, 7, 9, 11, 13 or 15, or fragment thereof, are contemplated. Moreover, nucleic acid molecules encoding Fndc5 proteins from different species, and thus have a nucleotide sequence that differs from the Fndc5 sequences of SEQ ID NOs: 1, 3 5, 7, 9, 11, 13 or 15 are also intended to be within the scope of the present invention. For example, rat or monkey Fndc5 cDNA may be identified based on the nucleotide sequence of a human and/or mouse Fndc5.

In one embodiment, nucleic acid molecule(s) encompassed by the present invention encode a protein or portion thereof that includes an amino acid sequence sufficiently homologous to an amino acid sequence of SEQ ID NO: 2, 4, 6, 8, 10, 12 or 14, or fragment thereof, such that the protein or portion thereof increases one or more of the following biological activities: 1) expression or activity of Fndc5 or a biologically active fragment thereof (e.g., irisin); 2) activity-induced immediate-early gene expression of Fndc5 or a biologically active fragment thereof (e.g., irisin); 3) preventing or reducing degeneration of dopaminergic (DA) neurons, preventing or ameliorating at least one motor deficit and/or preventing or ameliorating at least one symptom of cognitive dysfunction or dementia in a subject in need thereof, or 4) reduce the level or amount of α-synuclein in the cells.

As used herein, the language “sufficiently homologous” refers to proteins or portions thereof that have amino acid sequences that include a minimum number of identical or equivalent (e.g., an amino acid residue that has a similar side chain as an amino acid residue in SEQ ID NO: 2, 4, 6, 8, 10, 12 or 14, or fragment thereof) amino acid residues to an amino acid sequence of SEQ ID NO: 2, 4, 6, 8, 10, 12 or 14, or fragment thereof, such that the protein or portion thereof increases one or more of the following biological activities: 1) expression or activity of Fndc5 or a biologically active fragment thereof (e.g., irisin); 2) activity-induced immediate-early gene expression of Fndc5 or a biologically active fragment thereof (e.g., irisin); 3) preventing or reducing degeneration of dopaminergic (DA) neurons, preventing or ameliorating at least one motor deficit and/or preventing or ameliorating at least one symptom of cognitive dysfunction or dementia in a subject in need thereof, or 4) reduce the level or amount of α-synuclein in the cells.

In another embodiment, the protein is at least about 50%, at least about 60%, at least about 70%, 75%, 80%, 85%, 90%, 91%, 92%, 93%, 94%, 95%, 96%, 97%, 98%, 99% or more homologous to the entire amino acid sequence of SEQ ID NO: 2, 4, 6, 8, 10, 12 or 14, or fragment thereof, or a fragment thereof.

Portions of proteins encoded by Fndc5 or irisin nucleic acid molecules are biologically active portions of Fndc5. As used herein, the term “biologically active portion” is intended to include a portion, e.g., a domain/motif, of Fndc5 that has one or more of the biological activities of the full-length Fndc5 protein, such as listed above.

Standard binding assays, e.g., immunoprecipitations and yeast two-hybrid assays, as described herein, or functional assays, e.g., RNAi or overexpression experiments, may be performed to determine the ability of Fndc5 or a biologically active fragment thereof (e.g., irisin) to maintain a biological activity of the full-length Fndc5 protein.

The present invention further encompasses nucleic acid molecules that differ from the nucleotide sequence shown in SEQ ID NO: 1, 3, 5, 7, 9, 11, 13 or 15, or fragment thereof due to degeneracy of the genetic code and thus encode the same Fndc5 or a biologically active fragment thereof (e.g., irisin) as that encoded by the nucleotide sequence shown in SEQ ID NO: 1, 3, 5, 7, 9, 11, 13 or 15, for fragment thereof. In another embodiment, an isolated nucleic acid molecule encompassed by the present invention has a nucleotide sequence encoding a protein having an amino acid sequence shown in SEQ ID NO: 2, 4, 6, 8, 10, 12 or 14, or fragment thereof, or fragment thereof, or a protein having an amino acid sequence that is at least about 70%, 75%, 80%, 85%, 90%, 91%, 92%, 93%, 94%, 95%, 96%, 97%, 98%, 99% or more homologous to the amino acid sequence of SEQ ID NO: 2, 4, 6, 8, 10, 12 or 14, or fragment thereof, or a fragment thereof, or differs by at least 1, 2, 3, 5 or 10 amino acids but not more than 30, 20, 15 amino acids from SEQ ID NO: 2, 4, 6, 8, 10, 12 or 14. In another embodiment, a nucleic acid encoding an Fndc5 or irisin polypeptide consists of nucleic acid sequence encoding a portion of a full-length Fndc5 or irisin fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

It will be appreciated by those skilled in the art that DNA sequence polymorphisms that lead to changes in the amino acid sequences of Fndc5 or a biologically active fragment thereof (e.g., irisin) may exist within a population (e.g., a mammalian population, e.g., a human population). Such genetic polymorphism in the Fndc5 gene may exist among individuals within a population due to natural allelic variation. As used herein, the terms “gene” and “recombinant gene” refer to nucleic acid molecules comprising an open reading frame encoding an Fndc5 or irisin protein, such as a mammalian, e.g., human, Fndc5 or a biologically active fragment thereof (e.g., irisin). Such natural allelic variations can typically result in 1-5% variance in the nucleotide sequence of the Fndc5 gene. Any and all such nucleotide variations and resulting amino acid polymorphisms in Fndc5 or a biologically active fragment thereof (e.g., irisin) that are the result of natural allelic variation and that do not alter the functional activity of Fndc5 or a biologically active fragment thereof (e.g., irisin) are intended to be within the scope encompassed by the present invention. Moreover, nucleic acid molecules encoding Fndc5 or a biologically active fragment thereof (e.g., irisin) from other species, and thus that have a nucleotide sequence that differs from the human or mouse sequences of SEQ ID NO: 1, 3, 5, or 7, are intended to be within the scope encompassed by the present invention. Nucleic acid molecules corresponding to natural allelic variants and homologues of the human or mouse cDNAs of Fndc5 or a biologically active fragment thereof (e.g., irisin) encompassed by the present invention may be isolated based on their homology to the human or mouse nucleic acid sequences of Fndc5 or a biologically active fragment thereof (e.g., irisin) disclosed herein using the human or mouse cDNA, or a portion thereof, as a hybridization probe according to standard hybridization techniques under stringent hybridization conditions (as described herein).

In addition to naturally-occurring allelic variants of the sequence of Fndc5 or a biologically active fragment thereof (e.g., irisin) that may exist in the population, the skilled artisan will further appreciate that changes may be introduced by mutation into the nucleotide sequence of SEQ ID NO: 1, 3, 5, 7, 9, 11, 13 or 15, or fragment thereof, thereby leading to changes in the amino acid sequence of the encoded Fndc5 or a biologically active fragment thereof (e.g., irisin), without altering the functional ability of the Fndc5 or irisin protein. For example, nucleotide substitutions leading to amino acid substitutions at “non-essential” amino acid residues may be made in the sequence of SEQ ID NO: 1, 3, 5, 7, 9, 11, 13 or 15, or fragment thereof. A “non-essential” amino acid residue is a residue that may be altered from the wild-type sequence of Fndc5 or a biologically active fragment thereof (e.g., irisin) (e.g., the sequence of SEQ ID NO: 2, 4, 6, 8, 10, 12 or 14, or fragment thereof) without altering the activity of Fndc5 or a biologically active fragment thereof (e.g., irisin), whereas an “essential” amino acid residue is required for activity of Fndc5 or a biologically active fragment thereof (e.g., irisin). Other amino acid residues, however, (e.g., those that are not conserved or only semi-conserved between mouse and human) may not be essential for activity and thus are likely to be amenable to alteration without altering activity of Fndc5 or a biologically active fragment thereof (e.g., irisin). Furthermore, amino acid residues that are essential for functions of Fndc5 or a biologically active fragment thereof (e.g., irisin) related to the methods described herein, but not essential for Fndc5 functions related to thermogenesis, gluconeogenesis, cellular metabolism, and the like, are likely to be amenable to alteration.

Accordingly, another aspect encompassed by the present invention pertains to nucleic acid molecules encoding Fndc5 or a biologically active fragment thereof (e.g., irisin) that contain changes in amino acid residues that are not essential for activity of Fndc5 or a biologically active fragment thereof (e.g., irisin). Such proteins differ in amino acid sequence from SEQ ID NO: 2, 4, 6, 8, 10, 12 or 14, or fragment thereof, yet retain at least one of the activities of Fndc5 or a biologically active fragment thereof (e.g., irisin) described herein. In one embodiment, the isolated nucleic acid molecule comprises a nucleotide sequence encoding a protein, wherein the protein lacks one or more domains of Fndc5 or a biologically active fragment thereof (e.g., irisin) (e.g., a fibronectin, extracellular, signal peptide, hydrophobic, and/or C-terminal domain).

“Sequence identity or homology”, as used herein, refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared×100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from, from deletions or insertions in one of the sequences are counted as mismatches.

The comparison of sequences and determination of percent homology between two sequences may be accomplished using a mathematical algorithm. In one embodiment, the alignment may be performed using the Clustal Method. Multiple alignment parameters include GAP Penalty=10, Gap Length Penalty=10. For DNA alignments, the pairwise alignment parameters may beHtuple=2, Gap penalty=5, Window=4, and Diagonal saved=4. For protein alignments, the pairwise alignment parameters may beKtuple=1, Gap penalty=3, Window=5, and Diagonals Saved=5.

In one embodiment, the percent identity between two amino acid sequences is determined using the Needleman and Wunsch (J. Mol. Biol. (48):444-453 (1970)) algorithm that has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) that has been incorporated into the ALIGN program (version 2.0) (available online), using a PAM120 weight residue table, a gap length penalty of 12 and a gap penalty of 4.

An isolated nucleic acid molecule encoding Fndc5 or a biologically active fragment thereof (e.g., irisin) homologous to the protein of SEQ ID NO: 2, 4, 6, 8, 10, 12 or 14, or fragment thereof, may be created by introducing one or more nucleotide substitutions, additions or deletions into the nucleotide sequence of SEQ ID NO: 1, 3, 5, 7, 9, 11, 13 or 15, or fragment thereof, or a homologous nucleotide sequence such that one or more amino acid substitutions, additions or deletions are introduced into the encoded protein. Mutations may be introduced into SEQ ID NO: 1, 3, 5, 7, 9, 11, 13 or 15, or fragment thereof, or the homologous nucleotide sequence by standard techniques, such as site-directed mutagenesis and PCR-mediated mutagenesis. In one embodiment, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), bet 217-420 ranched side chains (e.g., threonine, valine, isoleucine) and aromatic side chains (e.g., tyrosine, phenylalanine, tryptophan, histidine). Thus, a predicted nonessential amino acid residue in Fndc5 or a biologically active fragment thereof (e.g., irisin) may be replaced with another amino acid residue from the same side chain family. Alternatively, in another embodiment, mutations may be introduced randomly along all or part of a coding sequence of Fndc5 or a biologically active fragment thereof (e.g., irisin), such as by saturation mutagenesis, and the resultant mutants may be screened for an activity of Fndc5 or a biologically active fragment thereof (e.g., irisin) described herein to identify mutants that retain activity of Fndc5 or a biologically active fragment thereof (e.g., irisin). Following mutagenesis of SEQ ID NO: 1, 3, 5, 7, 9, 11, 13 or 15, or fragment thereof, the encoded protein may be expressed recombinantly (as described herein) and the activity of the protein may be determined using, for example, assays described herein.

Levels of Fndc5 or a biologically active fragment thereof (e.g., irisin) may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In some embodiments, levels of Fndc5 or a biologically active fragment thereof (e.g., irisin) are ascertained by measuring gene transcript (e.g., mRNA), by a measure of the quantity of translated protein, or by a measure of gene product activity. Expression levels may be monitored in a variety of ways, including by detecting mRNA levels, protein levels, or protein activity, any of which may be measured using standard techniques. Detection can involve quantification of the level of gene expression (e.g., genomic DNA, cDNA, mRNA, protein, or enzyme activity), or, alternatively, may be a qualitative assessment of the level of gene expression, in particular in comparison with a control level. The type of level being detected will be clear from the context.

In a particular embodiment, mRNA expression level of Fndc5 or a biologically active fragment thereof (e.g., irisin) may be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA may be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well-known to those of skill in the art, such as, for example, the single-step RNA isolation process of Chomczynski (1989, U.S. Pat. No. 4,843,155).

The isolated mRNA may be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe may be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding Fndc5 or a biologically active fragment thereof (e.g., irisin). Other suitable probes for use in diagnostic assays encompassed by the present invention are described herein. Hybridization of an mRNA with the probe indicates that Fndc5 is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrix™ gene chip array. A skilled artisan can readily adapt known mRNA detection methods for use in detecting the level of the Fndc5 mRNA expression levels.

An alternative method for determining mRNA expression level of Fndc5 or a biologically active fragment thereof (e.g., irisin) in a sample involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to the Fndc5 mRNA.

As an alternative to making determinations based on the absolute expression level of Fndc5 or a biologically active fragment thereof (e.g., irisin), determinations may be based on the normalized expression level of Fndc5 or a biologically active fragment thereof (e.g., irisin). Expression levels are normalized by correcting the absolute expression level of Fndc5 or a biologically active fragment thereof (e.g., irisin) by comparing its expression to the expression of a gene that is not Fndc5 or a biologically active fragment thereof (e.g., irisin), e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of Fndc5 or a biologically active fragment thereof (e.g., irisin) may also be detected and/or quantified by detecting or quantifying the expressed polypeptide. The Fndc5 or a biologically active fragment thereof (e.g., irisin) may be detected and quantified by any of a number of means well-known to those of skill in the art. These may include analytic biochemical methods such as electrophoresis, capillary electrophoresis, high performance liquid chromatography (HPLC), thin layer chromatography (TLC), hyper-diffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like. A skilled artisan can readily adapt known protein/antibody detection methods for use in determining whether cells express Fndc5 or a biologically active fragment thereof (e.g., irisin).

Also provided are soluble, purified and/or isolated forms of Fndc5 or a biologically active fragment thereof (e.g., irisin). Hereinafter, irisin and fragments thereof will be considered to be encompassed within the term “fragments of Fndc5.”

In one aspect, a polypeptide of Fndc5 or a biologically active fragment thereof (e.g., irisin) may comprise a full-length Fndc5 amino acid sequence or a full-length Fndc5 amino acid sequence with 1 to about 20 conservative amino acid substitutions. The amino acid sequence of any Fndc5 polypeptide described herein may also be at least 50, 55, 60, 65, 70, 75, 80, 85, 90, 91, 92, 93, 94, 95, 96, 97, 98, 99, or 99.5% identical to an Fndc5 polypeptide sequence of interest, described herein, well-known in the art, or a fragment thereof. In addition, any Fndc5 polypeptide, or fragment thereof, described herein increases one or more of the following biological activities: 1) expression or activity of Fndc5 or a biologically active fragment thereof (e.g., irisin); 2) activity-induced immediate-early gene expression of Fndc5 or a biologically active fragment thereof (e.g., irisin); 3) preventing or reducing degeneration of dopaminergic (DA) neurons, preventing or ameliorating at least one motor deficit and/or preventing or ameliorating at least one symptom of cognitive dysfunction in a subject in need thereof, or 4) reduce the level or amount of α-synuclein in the cells. In another aspect, the present invention contemplates a composition comprising an isolated polypeptide of Fndc5 or a biologically active fragment thereof (e.g., irisin) and less than about 25%, or alternatively 15%, or alternatively 5%, contaminating biological macromolecules or polypeptides.

The present invention further provides compositions related to producing, detecting, or characterizing an Fndc5 or a biologically active fragment thereof (e.g., irisin), such as nucleic acids, vectors, host cells, and the like. Such compositions may serve as compounds that modulate an expression and/or activity of Fndc5 or a biologically active fragment thereof (e.g., irisin), such as antisense nucleic acids.

In some embodiments, a polypeptide of Fndc5 or a biologically active fragment thereof (e.g., irisin) comprises an amino acid modification, post-translational modification, and/or a heterologous an amino acid sequence, that stabilizes the polypeptide and/or increases its half-life. In certain embodiments, a polypeptide of Fndc5 or a biologically active fragment thereof (e.g., irisin) encompassed by the present invention may be a fusion protein containing a domain that increases its solubility and bioavailability and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, polypeptide of Fndc5 or a biologically active fragment thereof (e.g., irisin) encompassed by the present invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope encompassed by the present invention to include linker sequences between a polypeptide encompassed by the present invention and the fusion domain in order to facilitate construction of the fusion protein or to optimize protein expression or structural constraints of the fusion protein. In one embodiment, the linker is a linker described herein, e.g., a linker of at least 8, 9, 10, 15, 20 amino acids. The linker may be, e.g., an unstructured recombinant polymer (URP), e.g., a URP that is 9, 10, 11, 12, 13, 14, 15, 20 amino acids in length, i.e., the linker has limited or lacks secondary structure, e.g., Chou-Fasman algorithm. In another embodiment, the polypeptide may be constructed so as to contain protease cleavage sites between the fusion polypeptide and polypeptide encompassed by the present invention in order to remove the tag after protein expression or thereafter. Examples of suitable endoproteases, include, for example, Factor Xa and TEV proteases.

In some embodiments, a polypeptide of Fndc5 or a biologically active fragment thereof (e.g., irisin) may be fused to an antibody (e.g., IgG 1, IgG2, IgG3, IgG4) fragment (e.g., Fc polypeptides). Techniques for preparing these fusion proteins are known, and are described, for example, in WO 99/31241 and in Cosman et. al. (2001) Immunity 14:123-133. Fusion to an Fc polypeptide offers the additional advantage of facilitating purification by affinity chromatography over Protein A or Protein G columns.

In still another embodiment, a polypeptide of Fndc5 or a biologically active fragment thereof (e.g., irisin) may be labeled with a fluorescent label to facilitate their detection, purification, or structural characterization. In an exemplary embodiment, a polypeptide of Fndc5 or a biologically active fragment thereof (e.g., irisin) encompassed by the present invention may be fused to a heterologous polypeptide sequence that produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

Another aspect encompassed by the present invention pertains to the use of Fndc5 or a biologically active fragment thereof (e.g., irisin), as well as peptide fragments suitable for use as immunogens, to raise anti-Fndc5 antibodies. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of protein of Fndc5 or a biologically active fragment thereof (e.g., irisin) in which the protein is separated from cellular components of the cells in which it is naturally or recombinantly produced. In one embodiment, the language “substantially free of cellular material” includes preparations of protein of Fndc5 or a biologically active fragment thereof (e.g., irisin) having less than about 30% (by dry weight) of non-Fndc5 protein (also referred to herein as a “contaminating protein”), less than about 20% of non-Fndc5 protein, less than about 10% of non-Fndc5 protein, or less than about 5% non-Fndc5 protein. When the Fndc5 protein or biologically active portion thereof (e.g., irisin) is recombinantly produced, it is also substantially free of culture medium, i.e., culture medium represents less than about 20%, less than about 10%, or less than about 5% of the volume of the protein preparation. The language “substantially free of chemical precursors or other chemicals” includes preparations of protein of Fndc5 or a biologically active fragment thereof (e.g., irisin) in which the protein is separated from chemical precursors or other chemicals that are involved in the synthesis of the protein. In one embodiment, the language “substantially free of chemical precursors or other chemicals” includes preparations of protein of Fndc5 or a biologically active fragment thereof (e.g., irisin) having less than about 30% (by dry weight) of chemical precursors of non-Fndc5 chemicals, less than about 20% chemical precursors of non-Fndc5 chemicals, less than about 10% chemical precursors of non-Fndc5 chemicals, or less than about 5% chemical precursors of non-Fndc5 chemicals. In some embodiments, isolated proteins or biologically active portions thereof lack contaminating proteins from the same animal from which the Fndc5 protein is derived. Typically, such proteins are produced by recombinant expression of, for example, a human Fndc5 or a biologically active fragment thereof (e.g., irisin) in a non-human cell.

In some embodiments, the protein or portion thereof comprises an amino acid sequence that is sufficiently homologous to an amino acid sequence of SEQ ID NO: 2, 4, 6, 8, 10, 12 or 14, or fragment thereof, such that the protein or portion thereof maintains one or more of the following biological activities or, in complex, increases one or more of the following biological activities: 1) expression or activity of Fndc5 or a biologically active fragment thereof (e.g., irisin); 2) activity-induced immediate-early gene expression of Fndc5 or a biologically active fragment thereof (e.g., irisin); 3) preventing or reducing degeneration of dopaminergic (DA) neurons, preventing or ameliorating at least one motor deficit and/or preventing or ameliorating at least one symptom of cognitive dysfunction or dementia in a subject in need thereof, or 4) reduce the level or amount of α-synuclein in the cells. The portion of the protein is, in some embodiments, a biologically active portion as described herein. In another embodiment, the Fndc5 or a biologically active fragment thereof (e.g., irisin) has an amino acid sequence shown in SEQ ID NO: 2, 4, 6, 8, 10, 12 or 14, or fragment thereof, respectively, or an amino acid sequence that is at least about 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NO: 2, 4, 6, 8, 10, 12 or 14, or fragment thereof. In yet another embodiment, the Fndc5 or a biologically active fragment thereof (e.g., irisin) has an amino acid sequence that is encoded by a nucleotide sequence that hybridizes, e.g., hybridizes under stringent conditions, to the nucleotide sequence of SEQ ID NO:1, 3, 5, 7, 9, 11, 13 or 15, or fragment thereof, or a nucleotide sequence that is at least about 50%, at least about 60%, at least about 70%, at least about 80%, at least about 90%, or at least about 95% or more homologous to the nucleotide sequence shown in SEQ ID NO: 1, 3, 5, 7, 9, 11, 13 or 15, or fragment thereof. In some embodiments, the protein of Fndc5 or a biologically active fragment thereof (e.g., irisin) encompassed by the present invention also possess at least one of the Fndc5 biological activities, or activities associated with the complex, described herein. For example, a protein of Fndc5 or a biologically active fragment thereof (e.g., irisin) encompassed by the present invention includes an amino acid sequence encoded by a nucleotide sequence that hybridizes, e.g., hybridizes under stringent conditions, to the nucleotide sequence of SEQ ID NO:1, 3, 5, 7, 9, 11, 13 or 15, or fragment thereof and that increases one or more of the following biological activities: 1) expression or activity of Fndc5 or a biologically active fragment thereof (e.g., irisin); 2) activity-induced immediate-early gene expression of Fndc5 or a biologically active fragment thereof (e.g., irisin); 3) preventing or reducing degeneration of dopaminergic (DA) neurons, preventing or ameliorating at least one motor deficit, and/or preventing or ameliorating at least one symptom of cognitive dysfunction or dementia in a subject in need thereof, 4) reduce the level or amount of α-synuclein in the cells, or 5) expression or activity of at least one biomarker associated with an α-synucleinopathy or a cancer disclosed herein.

Biologically active portions of the Fndc5 protein include peptides comprising amino acid sequences derived from the amino acid sequence of the Fndc5 protein, e.g., the amino acid sequence shown in SEQ ID NO: 2, 4, 6, 8, 10, 12 or 14, or fragment thereof, or the amino acid sequence of a protein homologous to the Fndc5 protein, which include fewer amino acids than the full-length Fndc5 protein or the full-length protein that is homologous to the Fndc5 protein, and exhibit at least one activity of the Fndc5 protein, or complex thereof. Typically, biologically active portions (peptides, e.g., peptides that are, for example, 50, 55, 60, 65, 70, 75, 80, 85, 90, 95, 100 or more amino acids in length) comprise a domain or motif, e.g., signal peptide, extracellular domain, fibronectin domain, hydrophobic, and/or C-terminal domain). In an embodiment, the biologically active portion of the protein that includes one or more the domains/motifs described herein can decrease one of the following: 1) tremor at rest, such as a slight tremor in the hands or feet; 2) rigidity (stiffness) of limbs, neck, or shoulders; 3) difficulty balancing (postural instability); 4) slowness of movement or gradual loss of spontaneous movement (bradykinesia); 6) trouble standing after sitting; 7) stiffness in the limbs, and 8) moving more slowly. In an embodiment, the biologically active portion of the protein that includes one or more the domains/motifs described herein can decrease one of the following: confusion, poor motor coordination, loss of short-term or long-term memory, identity confusion, or impaired judgment. Moreover, other biologically active portions, in which other regions of the protein are deleted, may be prepared by recombinant techniques and evaluated for one or more of the activities described herein. In some embodiments, the biologically active portions of the Fndc5 protein include one or more selected domains/motifs or portions thereof having biological activity. In an exemplary embodiment, an Fndc5 fragment comprises and/or consists of about amino acids 29-140, 29-150, 30-140, 30-150, 73-140, 73-150, 1-140, 1-150, or any range in between residues 1 and 150 of SEQ ID NO:2. In another embodiment, an Fndc5 fragment consists of a portion of a full-length Fndc5 fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

Proteins of Fndc5 or a biologically active fragment thereof (e.g., irisin) may be produced by recombinant DNA techniques. For example, a nucleic acid molecule encoding the protein is cloned into an expression vector (as described above), the expression vector is introduced into a host cell (as described above) and the protein of Fndc5 or a biologically active fragment thereof (e.g., irisin) is expressed in the host cell. The protein of Fndc5 or a biologically active fragment thereof (e.g., irisin) can then be isolated from the cells by an appropriate purification scheme using standard protein purification techniques. Alternative to recombinant expression, a protein Fndc5 or a biologically active fragment thereof (e.g., irisin) may be synthesized chemically using standard peptide synthesis techniques. Moreover, native protein of Fndc5 or a biologically active fragment thereof (e.g., irisin) may be isolated from body fluids like plasma or cells, for example using an anti-Fndc5 antibody (described further below).

Also provided are chimeric or fusion proteins of Fndc5 or a biologically active fragment thereof (e.g., irisin), as described above. As used herein, a “chimeric protein” or “fusion protein” comprises a protein of Fndc5 or a biologically active fragment thereof (e.g., irisin) operatively linked to a non-Fndc5 polypeptide, for example, an Fc domain, an IgG1 Fc domain, an IgG2 Fc domain, an IgG3 Fc domain, and IgG4 Fc domain, a dimerization domain, an oligomerization domain, an agent that promotes plasma solubility, albumin, a signal peptide, a peptide tag, a 6-His tag, a thioredoxin tag, a hemaglutinin tag, a GST tag, or an OmpA signal sequence tag. A “Fndc5 polypeptide” refers to a polypeptide having an amino acid sequence corresponding to Fndc5, whereas a “non-Fndc5 polypeptide” refers to a polypeptide having an amino acid sequence corresponding to a protein that is not substantially homologous to the Fndc5 protein, respectively, e.g., a protein that is different from the Fndc5 protein and that is derived from the same or a different organism. Within the fusion protein, the term “operatively linked” is intended to indicate that the Fndc5 polypeptide and the non-Fndc5 polypeptide are fused in-frame to each other. The non-Fndc5 polypeptide may be fused to the N-terminus or C-terminus of the Fndc5 polypeptide, respectively. For example, in one embodiment the fusion protein is a Fndc5-GST and/or Fndc5-Fc fusion protein in which the Fndc5 sequences, respectively, are fused to the N-terminus of the GST or Fc sequences. Such fusion proteins can facilitate the purification, expression, and/or bioavailability of recombinant Fndc5. In another embodiment, the fusion protein is an Fndc5 protein containing a heterologous signal sequence at its C-terminus. In certain host cells (e.g., mammalian host cells), expression and/or secretion of Fndc5 may be increased through use of a heterologous signal sequence.

In some embodiments, a chimeric or fusion protein encompassed by the present invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers that give rise to complementary overhangs between two consecutive gene fragments that can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A nucleic acid encoding Fndc5 or a biologically active fragment thereof (e.g., irisin) may be cloned into such an expression vector such that the fusion moiety is linked in-frame to the Fndc5 protein.

Also provided are homologues of Fndc5 or a biologically active fragment thereof (e.g., irisin) that function as either an agonist (mimetic) or an antagonist of Fndc5 or the biologically active fragment thereof (e.g., irisin). In an embodiment, the agonists and antagonists stimulate or inhibit, respectively, a subset of the biological activities of the naturally occurring form of Fndc5 or a biologically active fragment thereof (e.g., irisin). Thus, specific biological effects may be elicited by treatment with a homologue of limited function. In one embodiment, treatment of a subject with a homologue having a subset of the biological activities of the naturally occurring form of the protein has fewer side effects in a subject relative to treatment with the naturally occurring form of Fndc5 or a biologically active fragment thereof (e.g., irisin).

Homologues of Fndc5 or a biologically active fragment thereof (e.g., irisin) may be generated by mutagenesis, e.g., discrete point mutation or truncation of the protein. As used herein, the term “homologue” refers to a variant form of Fndc5 or a biologically active fragment thereof (e.g., irisin) that acts as an agonist or antagonist of the activity of Fndc5 or a biologically active fragment thereof (e.g., irisin). An agonist can retain substantially the same, or a subset, of the biological activities of the protein. An antagonist of the protein can inhibit one or more of the activities of the naturally occurring form of the protein, by, for example, competitively binding to a downstream or upstream member of the Fndc5 cascade, which includes Fndc5 or a biologically active fragment thereof (e.g., irisin). Thus, mammalian Fndc5 or a biologically active fragment thereof (e.g., irisin), and homologues thereof, encompassed by the present invention may be, for example, either positive or negative regulators of neuronal cell function.

In an alternative embodiment, homologues of Fndc5 or a biologically active fragment thereof (e.g., irisin) may be identified by screening combinatorial libraries of mutants, e.g., truncation mutants, of Fndc5 or a biologically active fragment thereof (e.g., irisin) for agonist or antagonist activity. In one embodiment, a variegated library of variants of Fndc5 or a biologically active fragment thereof (e.g., irisin) is generated by combinatorial mutagenesis at the nucleic acid level and is encoded by a variegated gene library. A variegated library of variants of Fndc5 or a biologically active fragment thereof (e.g., irisin) may be produced by, for example, enzymatically ligating a mixture of synthetic oligonucleotides into gene sequences such that a degenerate set of potential sequences of Fndc5 or a biologically active fragment thereof (e.g., irisin) is expressible as individual polypeptides, or alternatively, as a set of larger fusion proteins (e.g., for phage display) containing the set of sequences therein. There are a variety of methods that may be used to produce libraries of potential homologues of Fndc5 or a biologically active fragment thereof (e.g., irisin) from a degenerate oligonucleotide sequence. Chemical synthesis of a degenerate gene sequence may be performed in an automatic DNA synthesizer, and the synthetic gene then ligated into an appropriate expression vector. Use of a degenerate set of genes allows for the provision, in one mixture, of all of the sequences encoding the desired set of potential sequences of Fndc5 or a biologically active fragment thereof (e.g., irisin). Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of Fndc5 may be used to generate a variegated population of Fndc5 fragments for screening and subsequent selection of homologues of Fndc5 or a biologically active fragment thereof (e.g., irisin). In one embodiment, a library of coding sequence fragments may be generated by treating a double stranded PCR fragment of a coding sequence of Fndc5 or a biologically active fragment thereof (e.g., irisin) with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with Si nuclease, and ligating the resulting fragment library into an expression vector. By this method, an expression library may be derived that encodes N-terminal, C-terminal and internal fragments of various sizes of the Fndc5 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of homologues of Fndc5 or a biologically active fragment thereof (e.g., irisin). The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, may be used in combination with the screening assays to identify Fndc5 homologues (Arkin and Youvan (1992) Proc. Natl. Acad. Sci. U.S.A. 89:7811-7815; Delagrave et al. (1993) Protein Engineering 6:327-331).

In addition, useful host cells and vectors are described supra for expressing desired nucleic acids and proteins for use according to the methods described herein.

### EXEMPLIFICATION

This invention is further illustrated by the following examples, which should not be construed as limiting.

### Example 1: Materials and Methods for Example 2

### Animal

C57BL/6 WT mice used in experiments were obtained from the Jackson Laboratories (Bar Harbor, ME). All housing, breeding, and procedures were performed according to the NIH Guide for the Care and Use of Experimental Animals and approved by Johns Hopkins University Animal Care and Use Committee.

### Preparation of α-Synuclein PFF

Recombinant mouse α-synuclein proteins were purified as previously described (T. I. Kam et al., Poly(ADP-ribose) drives pathologic α-synuclein neurodegeneration in Parkinson's disease. Science 362 (2018)) and bacterial endotoxins were removed by Toxineraser endotoxin removal kit (GeneScript). For generation of α-synuclein PFF, α-synuclein proteins were constantly agitated with a thermomixer (1,000 rpm at 37° C.) (Eppendorf, Hamburg, Germany) for 7 days and sonicated for 30 seconds (0.5 sec pulse on/off) at 10% amplitude (Branson Digital Sonifier, Danbury, CT) before use. Synthesis and purification of PAR polymer were performed as described (E. B. Affar et al., Immunological determination and size characterization of poly(ADP-ribose) synthesized in vitro and in vivo. Biochim Biophys Acta 1428, 137-146 (1999)). For generation of α-syn-biotin PFF, biotin was conjugated to recombinant α-synuclein with 2-3 molar ratio of biotin to α-synuclein using EZ-link Sulfo-NHS-LC-Biotin (Thermo Scientific, Grand Island, NY, USA) and α-synuclein-biotin PFF was prepared as described above.

### Preparation of Irisin and AAV-Irisin

Recombinant protein was prepared in mammalian cells as previously described (H. Kim et al., Irisin mediates effects on bone and fat via αV integrin receptors. Cell 175, 1756-1768.e17 (2018)). The Irisin-flag construct was prepared as previously described (M. R. Islam et al., Exercise hormone irisin is a critical regulator of cognitive function. Nat. Metab. 3, 1058-1070 (2021)). The pENN.AAV.CB7.CI.pm20d1flag.WPRE.rBG vector (Addgene plasmid #132682) replaced pm20d1flag using the PstI/HindIII restriction enzymes was used for cloning of the N-terminal part of mouse FNDC5 (signal peptide, amino acid residues 1-28) and irisin ORF plus flag-tag. The correct insertion of the signal peptide of mouse FNDC5 and irisin ORF was confirmed by Sanger sequencing. Packaging into the AAV (serotype 8) was performed at the Penn Vector Core. AAV8-GFP (pENN.AAV.CB7.CI.eGFP.WPRE.rBG) was used as control, generated by the Penn Vector Core, and obtained from Addgene (Addgene #105542).

### Stereotaxic Injection of α-Syn PFF and Intravenous Injection of AAV-Irisin

Two-month-old WT mice were deeply anesthetized with a mixture of ketamine (100 mg/kg) and xylazine (20 mg/kg) and fixed in a stereotaxic instrument. PBS or α-synuclein PFF (5 μg) was unilaterally injected into striatum (2 μl per hemisphere at 0.4 l/min) [anteroposterior (AP)=+2.0 mm, mediolateral (ML)=+2.0 mm, dorsoventral (DV)=+2.8 mm from bregma]. After the injection, the needle was maintained for an additional 5 min for a complete absorption of the solution. After surgery, animals were monitored and post-surgical care was provided. Two weeks after α-synuclein PFF injection, mice were injected with AAV8-GFP or AAV8-irisin-FLAG (100 μl of 1×10¹⁰ GC per mouse) into the tail vein. Behavioral tests were performed 6 months after injection and mice were euthanized for biochemical and histological analysis. For biochemical studies, tissues were immediately dissected and frozen at −80° C. For histological studies, mice were perfused with PBS and 4% PFA and brains were removed, followed by fixation in 4% PFA overnight and transfer to 30% sucrose for cryoprotection.

### Tissue Lysate Preparation and Western Blot Analysis

Dissected brain tissues were homogenized and prepared in lysis buffer [50 mM Tris-HCl (pH 7.4), 150 mM NaCl, 1 mM EDTA, 1% Triton x-100, 0.5% SDS, 0.5% sodium-deoxycholate, phosphatase inhibitor mixture I and II (Sigma-Aldrich, St. Louis, MO), and complete protease inhibitor mixture (Roche, Indianapolis, IN)], using a Diax 900 homogenizer (Sigma-Aldrich). The homogenates were rotated at 4° C. for 30 min for complete lysis, centrifuged at 15,000×g for 20 min and the supernatants were used for further analysis. Protein levels were quantified using the BCA assay (Pierce, Rockford, IL), samples were separated using SDS-polyacrylamide gels and transferred onto nitrocellulose membranes. The membranes were blocked with 5% non-fat milk in TBS-T (Tris-buffered saline with 0.1% Tween-20) for 1 h, subjected to immunoblotting using indicated primary antibodies and incubated with appropriate HRP-conjugated secondary antibodies (Cell signaling, Danvers, MA).

TABLE 2
The list of antibodies used in this study.

| Antibodies | Source | Identifier | Dilution |
|---|---|---|---|
| α-syn | BD Bioscience | 610787 | 1:1,000 (WB) |
| p-α-syn (ser129) | Biolegend | 825701 | 1:500 (IF) |
| p-α-syn (ser129) | Cell Signaling | 23706 | 1:1,000 (WB) |
| TH | Novus Biologicals | NB300-109 | 1:2,000 (WB) |
| | | | 1:1,000 (IHC) |
| DAT | Sigma | D6944 | 1:1,000 (WB) |
| Streptavidin-HRP | Thermo Fisher | N504 | 1:2,000 (WB) |
| Rab7 | Cell Signaling | 2094S | 1:1,000 (WB) |
| Lamp2 | Abcam | ab13524 | 1:1,000 (WB) |
| HSP60 | Cell Signaling | 12165 | 1:1,000 (WB) |
| β-actin-HRP | Sigma | A3854 | 1:20,000 (WB) |

### Measurement of Irisin Accumulation in the Brain

C57BL/6 mice were intravenously injected with purified irisin-His (1 mg/kg) for 1 hour. After collection of plasma, mice were perfused with PBS and brains were immediately removed. Levels of irisin-His in plasma and brain lysates were determined by the His tag ELISA detection kit (GenScript) according to manufacturer specifications.

### Endosome and Exosome Enrichment

Endosomes were enriched to detect internalized α-synuclein-biotin PFF as previously described (X. Mao et al., Pathological α-synuclein transmission initiated by binding lymphocyte-activation gene 3. Science 353 (2016)). Primary cultured neurons were incubated with α-synuclein-biotin PFF for 2 hours, followed by adding trypsin to remove the membrane-bound α-synuclein-biotin PFF. Neurons were lysed using a syringe 20 times in lysis buffer [250 mM sucrose, 50 mM Tris-HCl (pH 7.4), 5 mM MgCl₂, 1 mM EDTA, 1 mM EGTA] with a complete protease inhibitor mixture (Roche). Endosome-enriched fractions were obtained by sequential centrifugation at 1000×g for 10 min, 16,000×g for 20 min, and 100,000×g for 60 min at 4° C. Exosomes were enriched to detect secreted α-synuclein as previously described (E. Emmanouilidou et al., Cell-produced α-synuclein is secreted in a calcium-dependent manner by exosomes and impacts neuronal survival. J Neurosci 30, 6838-6851 (2010)). The supernatants from primary cortical neurons treated with α-synuclein-biotin PFF for 24 h were collected and spun at 300×g for 10 min to remove cells. The supernatants were then sequentially centrifuged at 2000×g for 10 min, 10,000×g for 30 min, and 100,000×g for 90 min at 4° C. The last pellet containing exosomes was washed once with PBS and centrifuged again at 100,000×g for 90 min. The remaining pellet was resuspended with lysis buffer.

### Primary Neuronal Culture and Treatment

Primary cortical neurons from WT embryos were prepared as described previously (T. I. Kam et al., FcgammaRIIb-SHIP2 axis links Abeta to tau pathology by disrupting phosphoinositide metabolism in Alzheimer's disease model. Elife 5 (2016)). Briefly, the primary cortical neurons were cultured at embryonic day 16 in neurobasal media supplemented with B-27, 0.5 mM L-glutamine, penicillin and streptomycin (Invitrogen, Carlsbad, CA). At 7 days in vitro (DIV), indicated concentration of irisin was pre-incubated for 1 hour and α-synuclein PFF were further incubated for indicated times followed by cell death assay or biochemical experiments. The neuron culture media was replaced with fresh medium alone or including irisin every 3-4 days.

Specifically, cultured neurons were treated with 5-Fluorodeoxyuridine (5-FDU) (MP Biomedicals) at a final concentration of 10 μM only one time. Therefore, half the medium was exchanged with fresh neurobasal medium containing 20 μM 5-FDU once 24 hours after seeding. Cells were then maintained in neurobasal media containing B-27, 0.5 mM L-glutamine, penicillin, and streptomycin (Invitrogen). Half the neurobasal medium was changed every 3-4 d, and therefore the 5-FDU was diluted upon subsequent medium changes. For Irisin treatments, the protein was added to the culture medium to the indicated final concentrations for 1 hour on day 7 in vitro (7 DIV). After 1 hour preincubation with irisin, half of the cell culture medium was replaced with fresh medium containing α-syn PFFs plus irisin. For the subsequent irisin treatments, half the cell culture medium was replaced with fresh medium alone or medium containing irisin every 3-4 days. Sequential extraction of Triton X-100-soluble and insoluble α-syn was performed as described previously (J. L. Guo et al., Distinct α-synuclein strains differentially promote tau inclusions in neurons. Cell 154, 103-117 (2013)). Neuronal lysates were prepared in Triton lysis buffer (50 mM Tris, [pH 7.6]150 mM NaCl, 1% Triton X-100, phosphatase inhibitor mixture I and II [Sigma-Aldrich], and complete protease inhibitor mixture [Roche]). The Triton-soluble fraction was collected from the supernatants after sonication followed by centrifugation at 100,000×g for 30 min at 4° C. The remaining pellets were washed in Triton lysis buffer and resuspended into sodium dodecyl sulfate (SDS) lysis buffer (50 mM Tris, [pH 7.6]150 mM NaCl, 2% SDS, phosphatase inhibitor mixture I and II [Sigma-Aldrich], and complete protease inhibitor mixture [Roche]), sonicated, and centrifuged at 100,000×g for 30 min at room temperature. The supernatants were used as the Triton-insoluble fraction.

### Cell Death Assessment

Primary cultured cortical neurons were treated with 5 μg/ml of α-synuclein PFF in the presence or absence of irisin for 14 days. Cell death was determined by staining with 7 μM Hoechst 33342 and 2 μM propidium iodide (PI) (Invitrogen). Images were taken and counted by Zeiss microscope equipped with automated computer assisted software (Axiovision 4.6, Carl Zeiss, Dublin, CA).

### Immunohistochemistry and Immunofluorescence

Mice were perfused with PBS and 4% PFA and brains were removed and transfer to 30% sucrose for cryoprotection. Immunohistochemistry (IHC) was performed on 40 μm thick serial brain sections. For histological studies, free-floating sections were blocked with 10% goat serum in PBS with 0.2% Triton X-100 and incubated with TH antibodies followed by incubation with biotin-conjugated anti-rabbit antibody. ABC reagent (Vector laboratories, Burlingame, CA) was added after washing and the sections were developed using SigmaFast DAB peroxidase substrate (Sigma-Aldrich). Sections were counterstained with Nissl (0.09% thionin). For the quantification, both TH- and Nissl-positive DA neurons from the SNpc region were counted by an investigator who was blind to treatment condition with randomly allocated groups through optical fractionators, the unbiased method for cell counting, using a computer-assisted image analysis system consisting of an Axiophot photomicroscope (Carl Zeiss) equipped with a computer controlled motorized stage (Ludl Electronics, Hawthorne, NY), a Hitachi HV C20 camera, and Stereo Investigator software (MicroBright-Field, Williston, VT). The total number of TH-stained neurons and Nissl counts were analyzed as previously described (S. S. Karuppagounder et al., The c-Abl inhibitor, nilotinib, protects dopaminergic neurons in a preclinical animal model of Parkinson's disease. Sci Rep 4, 4874 (2014)). For immunofluorescent studies in primary cultures, Ser129 p-α-synuclein antibodies were incubated followed by Alexa-fluor 488-conjugated secondary antibodies (Invitrogen). The fluorescent images were acquired by confocal scanning microscopy (LSM710, Carl Zeiss). All the images were processed by the Zen software (Carl Zeiss). The selected area in the signal intensity range of the threshold was measured using ImageJ software.

### Dopamine and Derivatives Measurement Using HPLC

The striatum dissected from the brain were sonicated in ice-cold perchloric acid (0.01 mM) containing 0.01% EDTA. The homogenates were centrifuged at 15,000×g for 30 min at 4° C. and the debris in supernatants were removed using a 0.2 m filter. Dopamine levels were analyzed using the HPLC column (3 mm×150 mm, C-18 reverse phase column, Atlantis T3 3 μm, Thermo Scientific) with a dual channel coulochem III electrochemical detector (Model 5300, ESA Inc.). The 60 ng of 3,4-dihydroxybenzylamine (DHBA) was used as an internal standard. The values were normalized to protein concentrations measured from a BCA protein assay kit (Pierce) and the data were expressed in ng/mg protein.

### Mass Spectrometry Sample Preparation

Primary cortical neurons were pre-incubated with irisin (50 ng/ml) for 1 h and α-synuclein PFF were further incubated for 1 or 4 days. The lysates were used for quantitative protein Mass Spectrometry analysis, with isobaric tagging using the TMT method. Soluble lysates were extracted from cells using a buffer comprised of 1% Triton X-100 in Tris buffer (50 mM Tris, 150 mM NaCl, pH 7.4) and protease inhibitors. Protein concentration was measured and 15 g of protein from each sample was prepared for MS analysis. Samples were diluted with an equal volume of the buffer (400 mM EPPS pH 8.5, 0.5% SDS, 10 mM Tris(2-carboxyethyl)phosphine hydrochloride) and incubated for 10 min at room temperature. Iodoacetimide (final concentration of 10 mM) was added and further incubated for 25 min in the dark, followed by DTT (final concentration of 10 mM) was added. A buffer exchange was carried out using a modified SP3 protocol as previously reported (C. S. Hughes et al., Ultrasensitive proteome analysis using paramagnetic bead technology. Mol. Syst. Biol. 10, 757 (2014); C. S. Hughes et al., Single-pot, solid-phase-enhanced sample preparation for proteomics experiments. Nat. Protoc. 14, 68-85 (2019)). Briefly, ˜250 g of each SpeedBead magnetic carboxylate modified particles (Cytiva; 45152105050250, 65152105050250) mixed at a 1:1 ratio were added to each sample. Samples were combined with ethanol to make a final ethanol concentration of at least 50% and incubated for 15 min with gentle shaking. After three washes washing with 80% ethanol, proteins were eluted from SP3 beads using 200 mM EPPS (pH 8.5) containing trypsin (ThermoFisher Scientific) and Lys-C (Wako) and digested overnight at 37° C. with vigorous shaking. Samples were combined with acetonitrile (final concentration of 33%) and then labeled with TMTpro-18plex reagents (˜65 μg) (ThermoFisher Scientific). After confirmation of >97% labeling, excess TMTpro reagents were quenched by addition of hydroxylamine (final concentration of 0.3%). Acetonitrile was removed from the pooled samples by vacuum centrifugation for 1 hour and acidified using formic acid. The peptides were de-salted using a Sep-Pak Vac 50 mg tC18 cartridge (Waters) and eluted in 70% acetonitrile, 1% formic acid. Dried peptides were resuspended in 10 mM ammonium bicarbonate (pH 8.0) and 5% acetonitrile. Twenty-four fractions were collected after fractionation by basic pH reverse phase HPLC were dried, resuspended in 5% acetonitrile and 1% formic acid, and de-salted by stage-tip. The peptides were eluted in 70% acetonitrile and 1% formic acid, dried, and finally resuspended in 5% acetonitrile and 5% formic acid. A total 11 of 24 fractions were analyzed by LC-MS/MS.

### Mass Spectrometry Data Acquisition

Data were acquired on an Orbitrap Eclipse mass spectrometer paired with a Proxeon EASY nLC 1000 LC pump. Prepared peptides were solubilized in 5% ACN/5% formic acid, loaded onto a C18 column (30 cm, 2.6 μm Accucore, 100 μm ID), and eluted over a 120-min gradient. High-field asymmetric-waveform ion mobility spectroscopy was used during data collection with compensation voltages (CVs) of −40 V, −60 V, and −80 V. MS1 precursor scans were acquired in the orbitrap with the following parameters: 120 K resolution, 4e5 AGC target, and a maximum of 50-ms injection time. The ion trap was used to collect MS2 scans (1 s per CV) using collisional induced dissociation fragmentation. MS2 scans were collected with the following settings: NCE 35%, 2e4 AGC target, maximum injection time 50 ms, isolation window 0.5 Da. Orbiter, a real-time search algorithm, was used to trigger MS3 quantification scans. These scans were acquired in the orbitrap with the following settings: 50,000 resolution, AGC of 2×10⁵-5×10⁵, injection time of 150 ms, HCD collision energy of 45%. Protein-level closeout was set to five peptides per protein per fraction for six fractions and two peptides per protein per fraction for five fractions (D. K. Schweppe et al., Full-featured, real-time database searching platform enables fast and accurate multiplexed quantitative proteomics. J. Proteome Res. 19, 2026-2034 (2020)).

### Mass Spectrometry Data Analysis

Raw files were converted to mzXMIL. Monocle was used to reassign monoisotopic peaks. Database searching used all mouse entries from Uniprot (July 2014) combined with all protein sequences in the reversed order. The sequences of frequent contaminant proteins were also included. Comet was used to perform the searches using a 50-ppm precursor ion tolerance and 1.0005 Da product ion tolerance. Static modifications were set as follows: TMTpro on lysine residues and peptide N termini (+304.2071 Da) and carbamidomethylation of cysteine residues (+57.0215 Da). Methionine oxidation (+15.9949 Da) was set as a variable modification.

Peptide-spectrum matches were filtered to a 1% false discovery rate (FDR) (J. E. Elias, S. P. Gygi, Target-decoy search strategy for increased confidence in large-scale protein identifications by mass spectrometry. Nat. Methods 4, 207-214 (2007)) using linear discriminant analysis (LDA) on each run as described previously (E. L. Huttlin et al., A tissue-specific atlas of mouse protein phosphorylation and expression. Cell 143, 1174-1189 (2010)). LDA used the following parameters: comet log expect, different sequence delta comet log expect (percent difference between the first hit and the next hit with a different peptide sequence), missed cleavages, length of peptide, charge state of peptide, mass accuracy of the precursor, and percentage of ions matched. In contrast to peptide-level FDR, which filtered each run separately, protein-level FDR was estimated at the level of the full dataset. For each protein, the posterior probabilities (as determined by LDA) for an individual peptide were multiplied to yield a protein-level probability estimate. Proteins were filtered to the target 1% FDR level, utilizing the Picked FDR method (M. M. Savitski, M. Wilhelm, H. Hahne, B. Kuster, M. Bantscheff, A scalable approach for protein false discovery rate estimation in large proteomic data sets. Mol. Cell. Proteomics 14, 2394-2404 (2015)).

In order to quantify reporter ions, a 0.003 Da window around the theoretical m z of each reporter ion was scanned, using the most intense m z. Reporter ion intensities were corrected for the isotopic impurities using specifications provided by the manufacturer. Only peptides with a summed signal-to-noise (across all channels) greater than 160 were included. TMTpro signal-to-noise values for individual peptides were summed to quantify proteins.

### Statistical Analysis or Proteomic Data

Statistical analysis was performed using Perseus (S. Tyanova et al., The Perseus computational platform for comprehensive analysis of (prote)omics data. Nat. Methods 13, 731-740 (2016)). Total quantified proteins were filtered to remove unreviewed TrREMBL sequences and proteins quantified using a single peptide. A permutation-based FDR was used to identify significant changes. The following settings within Perseus were used: FDR, 0.05; S0, 0.1; and number of randomizations, 250.

### Behavioral Tests

The behavioral deficits in α-synuclein PFF injected mice treated with AAV-irisin were assessed by the pole test and the grip strength test 1 week prior to sacrifice. All the experiments were performed by investigators who are blind to treatment condition and randomly allocated to groups.

Pole test. A metal rod (75 cm long with a 9 mm diameter) wrapped with bandage gauze was used as the pole. Before the actual test, the mice were trained for two consecutive days and each training session consisted of three test trials. Mice were placed on 7.5 cm from the top of the pole and the time to turn and total time to reach the base of the pole were recorded. The end of test was defined as placing all 4 paws on the base. The maximum cutoff time to stop the test and recording was 60 seconds. After each trial, the maze was cleaned with 70% ethanol.

Grip strength test. Neuromuscular function was measured by determining the maximal peak force developed by the mice using an apparatus (Bioseb, USA). Mice were placed onto a metal grid to grasp with either fore or both limbs that are recorded as ‘fore limb’ and ‘fore and hindlimb’, respectively. The tail was gently pulled and the force applied to the grid before the mice lose grip was recorded as the peak tension displayed in grams (g).

### Statistical Analysis

All data are represented as mean±s.e.m. with at least three independent experiments. Statistical analysis was performed using GraphPad Prism 7. Differences between two means and among multiple means were assessed by unpaired two-tailed student t test and ANOVA followed by Tukey's post hoc test, respectively. Assessments with P<0.05 were considered significant.

### Data Availability

The mass spectrometry data were deposited to the ProteomeXchange Consortium (PXD032670) (T-I. Kam et al., Amelioration of pathologic α-synuclein-induced Parkinson's disease by irisin. ProteomeXchange Consortium. Deposited 20 Mar. 2022).

### Example 2: Amelioration of Pathologic α-Synuclein-Induced Parkinson's Disease by Irisin

Irisin is a small polypeptide that is secreted by muscle and other tissues into the blood of mice and humans (P. Bostrom et al., A PGC1-alpha-dependent myokine that drives brown-fat-like development of white fat and thermogenesis. Nature 481, 463-468 (2012); M. P. Jedrychowski et al., Detection and Quantitation of Circulating Human Irisin by Tandem Mass Spectrometry. Cell Metab 22, 734-740 (2015)). The amino acid sequence is conserved 100% between mice and humans, suggesting a critical, conserved function. Importantly, the expression of irisin and its precursor protein FNDC5, are increased in muscle with many forms of exercise in rodents and humans. Irisin levels increase in the blood of humans with exercise training by Tandem Mass Spectrometry (M. P. Jedrychowski et al., Detection and Quantitation of Circulating Human Irisin by Tandem Mass Spectrometry. Cell Metab 22, 734-740 (2015)). In adipose cells, osteocytes and osteoclasts, integrin αV/β5 the major functioning receptor for irisin (H. Kim et al., Irisin Mediates Effects on Bone and Fat via alphaV Integrin Receptors. Cell 175, 1756-1768 e1717 (2018)).

Physical activity can possibly prevent and ameliorate the symptoms of multiple forms of neurodegeneration, including Alzheimer's Disease (AD) and PD (K. S. Bhalsing, M. M. Abbas, L. C. S. Tan, Role of Physical Activity in Parkinson's Disease. Ann Indian Acad Neurol 21, 242-249 (2018); B. M. Brown, J. J. Peiffer, R. N. Martins, Multiple effects of physical activity on molecular and cognitive signs of brain aging: can exercise slow neurodegeneration and delay Alzheimer's disease? Mol Psychiatry 18, 864-874 (2013); S. H. Choi et al., Combined adult neurogenesis and BDNF mimic exercise effects on cognition in an Alzheimer's mouse model. Science 361 (2018); I. Marques-Aleixo et al., Preventive and Therapeutic Potential of Physical Exercise in Neurodegenerative Diseases. Antioxid Redox Signal 34, 674-693 (2021)).

The effects of irisin on various models of neurodegenerationis shown herein. It is shown that elevated expression of FNDC5 in the liver via the use of adenoviral vectors, and elevations of irisin in the blood, stimulated an “exercise-like” program of gene expression in the hippocampus (C. D. Wrann et al., Exercise induces hippocampal BDNF through a PGC-1alpha/FNDC5 pathway. Cell Metab 18, 649-659 (2013)). Moreover, the expression of FNDC5 with these same viral vectors rescued memory deficits in a mouse model of AD (M. V. Lourenco et al., Exercise-linked FNDC5/irisin rescues synaptic plasticity and memory defects in Alzheimer's models. Nat Med 25, 165-175 (2019)). Most recently, irisin was shown to be the active moiety regulating cognitive function in four separate mouse models. Importantly, elevation of the blood levels of the mature, cleaved irisin was sufficient to improve cognitive function and reduce neuroinflammation in two distinct models of AD (M. R. Islam et al., Exercise hormone irisin is a critical regulator of cognitive function. Nat Metab 3, 1058-1070 (2021)). Furthermore, irisin itself crossed the blood-brain barrier (BBB), at least when the protein was produced from the liver with these AAV vectors.

Herein, the effects of irisin on the pathophysiology of Parkinson's disease are examined, using the α-synuclein preformed fibril (α-synuclein PFF) seeding model in culture and in vivo. Pathologic α-synuclein is thought to spread “prion-like” in the brains of patients with Parkinson's disease and certain other neurological disorders, where they cause neuronal death and dysfunction. Herein, it shown that irisin has powerful effects in preventing both the accumulation of pathologic α-syn and neuronal cell death in primary cell culture. Furthermore, elevation of blood irisin levels in mice normalizes the histological manifestations in the SNc and the Parkinson's disease-like symptomology involving movement and grip strength induced by intrastriatal injection of α-syn PFF. These data suggest the potential therapeutic value of irisin in Parkinson's disease and other neurodegenerative states that involve α-synuclein.

### Results

### Irisin Prevents the Formation of Pathologic α-Synuclein and Protects Neurons Against α-Syn PFF-Induced Neurotoxicity.

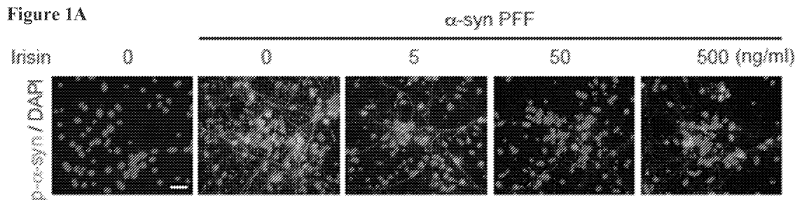

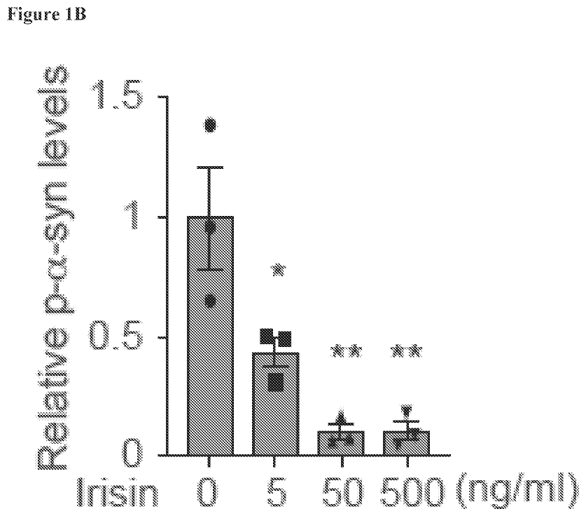

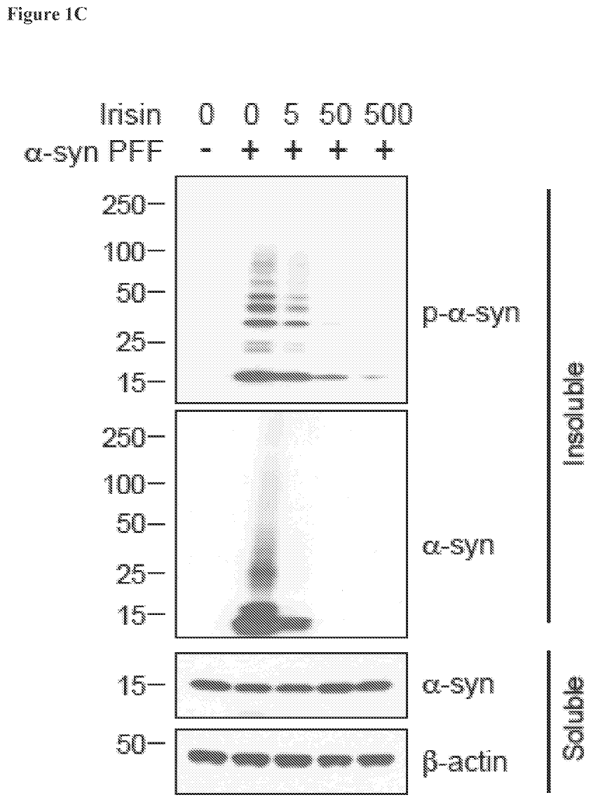

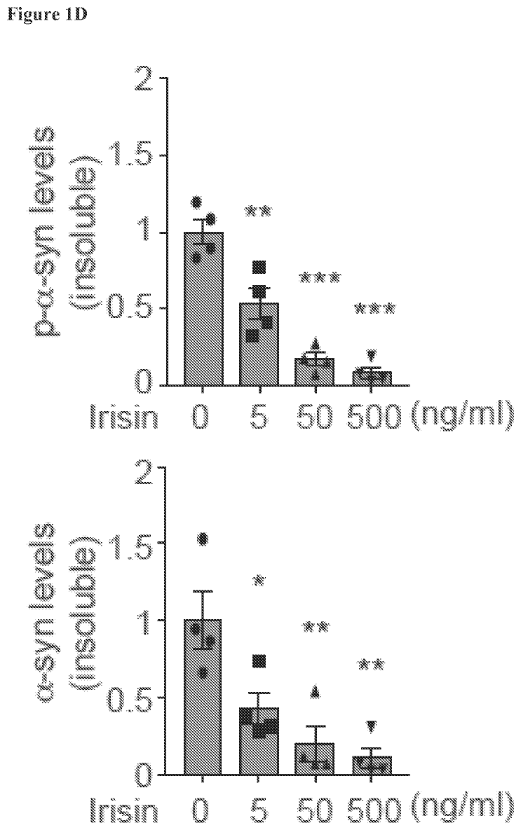

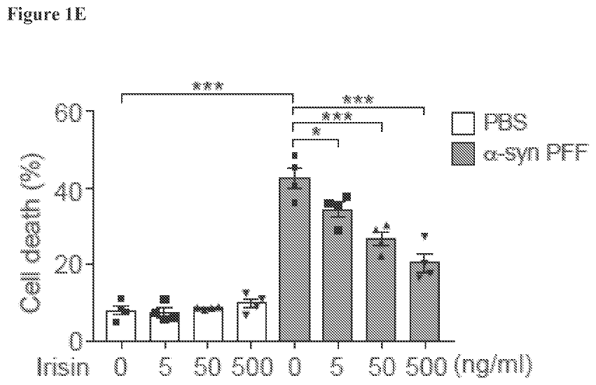

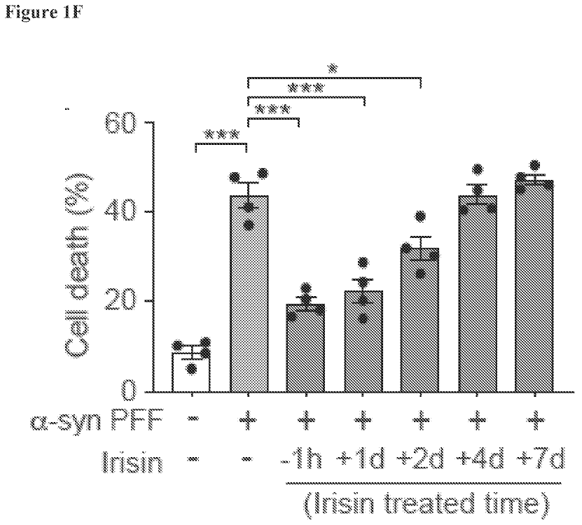

α-synuclein PFF administration to cortical neurons induces endogenous α-synuclein to misfold and become pathologic (T. I. Kam et al., Poly(ADP-ribose) drives pathologic α-synuclein neurodegeneration in Parkinson's disease. Science 362 (2018); K. C. Luk et al., Pathological α-synuclein transmission initiates Parkinson-like neurodegeneration in nontransgenic mice. Science 338, 949-953 (2012)) This transformation can be monitored by the phosphorylation of α-syn serine 129 (p-α-syn) (H. Fujiwara et al., α-Synuclein is phosphorylated in synucleinopathy lesions. Nat Cell Biol 4, 160-164 (2002). One hour pretreatment of cortical neurons with 5 ng/ml of irisin significantly reduced the levels p-a-synuclein and 50 and 500 ng/ml of irisin prevented the formation of p-α-syn as determined via immunocytochemistry (FIGS. 1A and 1B) and immunoblot analysis (FIGS. 1C and 1D) 7 days after the α-synuclein PFF administration. Irisin also prevented the accumulation of Triton X-100 insoluble p-α-synuclein and α-synuclein (FIGS. 1C and 1D). One hour pretreatment of cortical neurons with 5, 50 and 500 ng/ml of irisin prevented the death of cortical neurons induced by α-synuclein PFF as assessed 14 days after administration of α-synuclein PFF (FIG. 1E). In addition to 1 hour irisin pretreatment, irisin was able prevent the death of cortical neurons with 1 or 2 days after administration of α-syn PFF (FIG. 1F). Four and 7 day post-treatment with irisin had no effect on α-synuclein PFF-induced cell death (FIG. 1F). Collectively, these data indicated that irisin prevents the formation of pathologic α-synuclein and protects neurons against α-synuclein PFF-induced neurotoxicity.

### Irisin Inhibits the Internalization and Propagation of α-Syn.

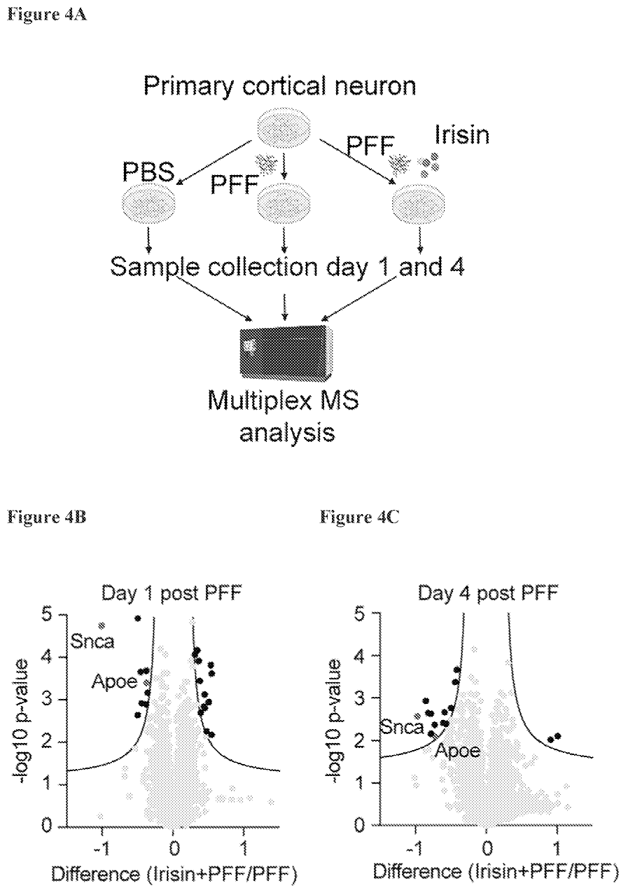

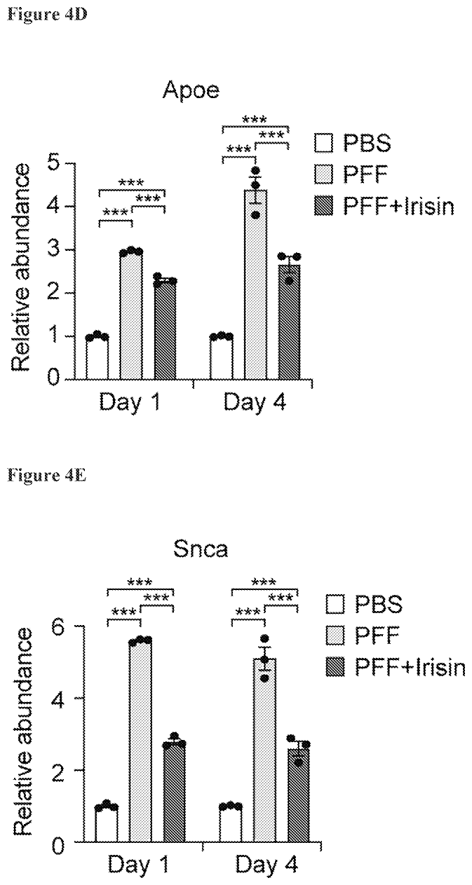

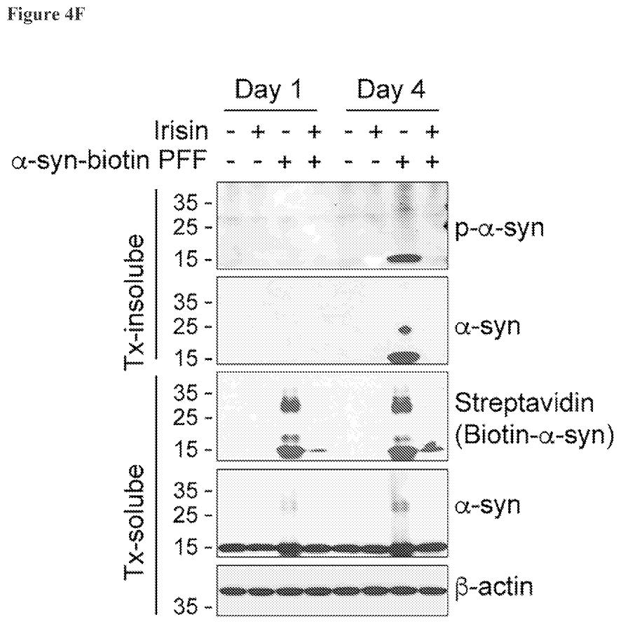

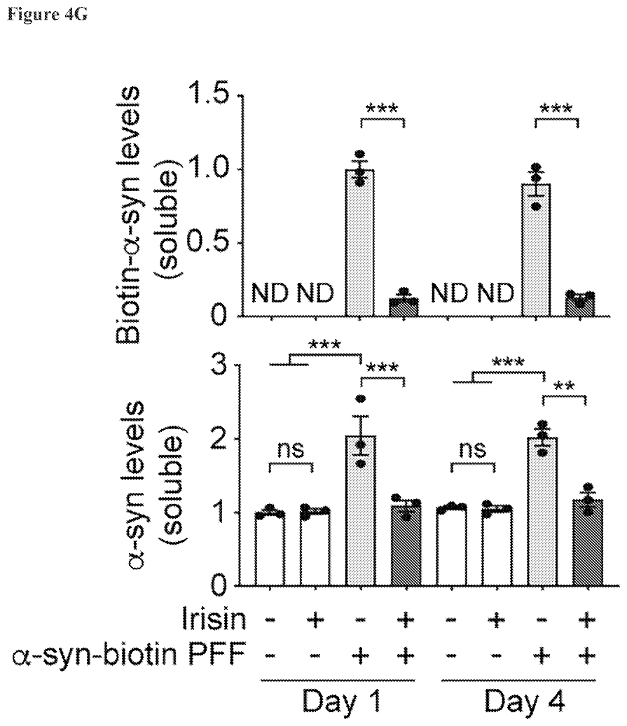

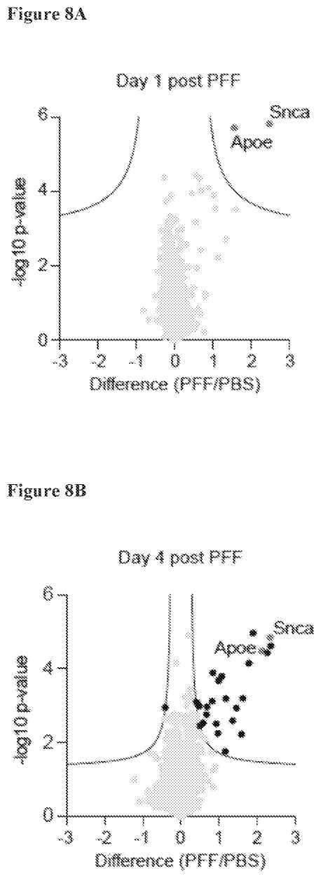

To determine the molecular pathways by which irisin might prevent α-syn PFF-induced neurodegeneration, the proteomes from primary cultured cortical neurons treated with α-syn PFF for 1 or 4 day in the absence and presence of irisin were analyzed by liquid chromatography-tandem mass spectrometry (LC-MS/MS) (FIG. 4A). Statistical analysis of the quantified proteins showed that 2 and 26 proteins were differentially regulated by α-syn PFF at 1 and 4 day after treatment, respectively (FIGS. 8A and 8B). Among them, 100% and 34.6% of proteins 1 and 4 day after α-syn PFF treatment were counter regulated by irisin (FIGS. 4B and 4C). Of note, irisin treatment significantly changed the abundance of 22 and 15 proteins 1 and 4 day after α-syn PFF treatment when compared to α-syn PFF-treated neurons only (FIGS. 4B and 4C). α-Syn PFF treatment significantly up-regulated the ApoE protein (FIG. 4D), whose F4 genotype in humans regulates α-syn pathology (A. A. Davis et al., APOE genotype regulates pathology and disease progression in synucleinopathy. Sci. Transl. Med 12, eaay3069 (2020)) and is associated with an increased risk of dementia in PD (J. Bras et al., Genetic analysis implicates APOE, SNCA and suggests lysosomal dysfunction in the etiology of dementia with Lewy bodies. Hum. Mol. Genet. 23, 6139-6146 (2014); I. F. Mata et al., APOE, MAPT, and SNCA genes and cognitive performance in Parkinson disease. JAMA Neurol. 71, 1405-1412 (2014)) and AD (J. Kim, J. M. Basak, D. M. Holtzman, The role of apolipoprotein E in Alzheimer's disease. Neuron 63, 287-303 (2009)). Irisin significantly down-regulated ApoE (FIG. 4D). Importantly, the α-syn protein itself, which increased after α-syn PFF administration showed a decrease following irisin treatment 1 and 4 day later (FIG. 4E). The levels of α-syn in Tx-soluble and Tx-insoluble fractions after treatment of cortical neurons with biotin-labeled α-syn PFF (α-syn-biotin PFF) and irisin were measured. Prior experiments have shown that α-syn-biotin enters neurons and templates endogenous α-syn to misfold and become pathogenic, in a manner similar to unlabeled α-syn PFF (X. Mao et al., Pathological α-synuclein transmission initiated by binding lymphocyte-activation gene 3. Science 353, aah3374 (2016)). One hour pre- and continuous treatment of cortical neurons with 50 ng/mL of irisin significantly reduced the levels of α-syn-biotin PFF in both the Tx-soluble fraction after 1 or 4 day and in the Tx-insoluble fraction 4 d after α-syn PFF administration (FIG. 4F and FIG. 4G). Endogenous α-syn levels paralleled the changes in α-syn-biotin (FIG. 4F and FIG. 4G). Pathologic formation of p-α-syn in the Tx-insoluble fraction starts 4 d after α-syn PFF administration (T. I. Kam et al., Poly(ADP-ribose) drives pathologic α-synuclein neurodegeneration in Parkinson's disease. Science 362, eaat8407 (2018)), which was prevented by irisin (FIG. 4F). Taken together, these data suggest that irisin may prevent the intracellular accumulation of a pSer129-positive pathologic form of α-syn by decreasing its internalization and aggregation.

### Irisin Enhances the Lysosomal Degradation of α-Synuclein PFF.

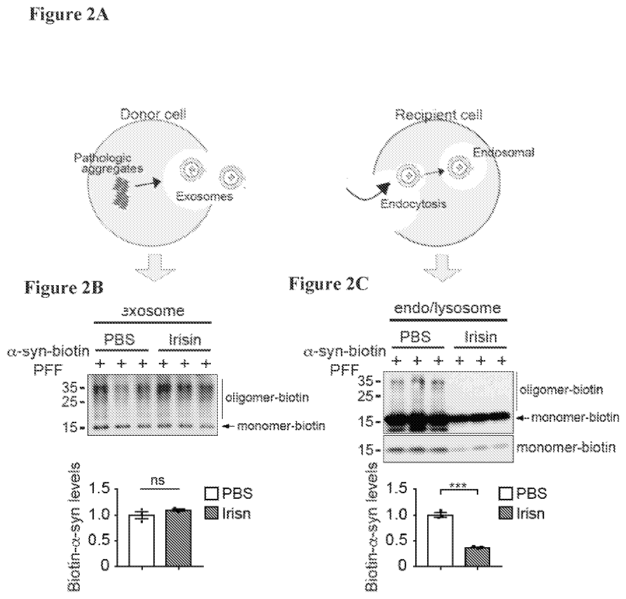

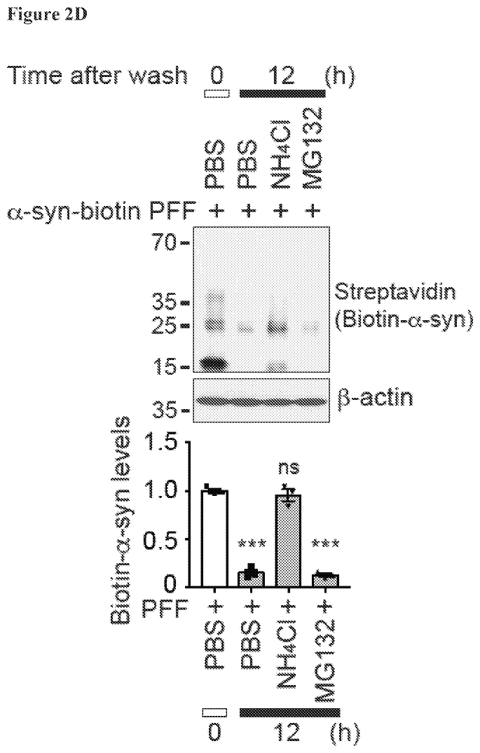

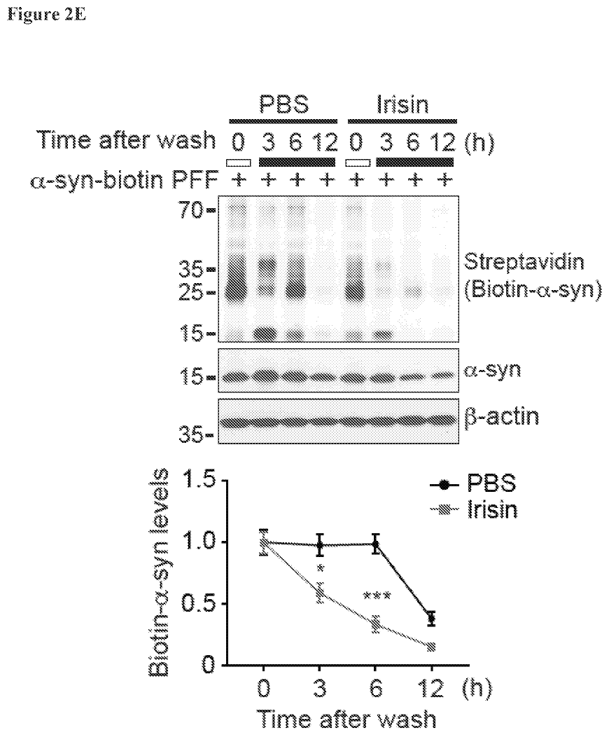

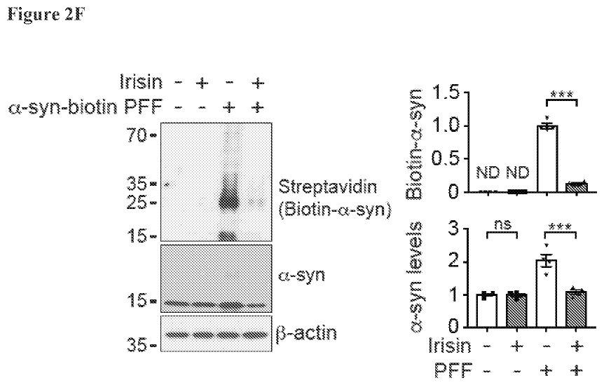

α-synuclein PFF are taken up into neurons via receptor mediated endocytosis, macropinocytosis or tunneling nanotubes (S. Abounit et al., Tunneling nanotubes spread fibrillar α-synuclein by intercellular trafficking of lysosomes. EMBO J 35, 2120-2138 (2016); B. B. Holmes et al., Heparan sulfate proteoglycans mediate internalization and propagation of specific proteopathic seeds. Proc Natl Acad Sci USA 110, E3138-3147 (2013); X. Mao et al., Pathological α-synuclein transmission initiated by binding lymphocyte-activation gene 3. Science 353 (2016); N. Uemura, M. T. Uemura, K. C. Luk, V. M. Lee, J. Q. Trojanowski, Cell-to-Cell Transmission of Tau and α-Synuclein. Trends Mol Med 26, 936-952 (2020) where they end up in endolysosome where they can template endogenous monomeric α-synuclein to misfold into pathologic α-synuclein (M. M. Apetri et al., Direct Observation of alpha-Synuclein Amyloid Aggregates in Endocytic Vesicles of Neuroblastoma Cells. PLoS One 11, e0153020 (2016); R. J. Karpowicz, Jr. et al., Selective imaging of internalized proteopathic alpha-synuclein seeds in primary neurons reveals mechanistic insight into transmission of synucleinopathies. J Biol Chem 292, 13482-13497 (2017); C. Masaracchia et al., Membrane binding, internalization, and sorting of alpha-synuclein in the cell. Acta Neuropathol Commun 6, 79 (2018). Once inside cells the α-synuclein PFF and the endogenous pathologic α-synuclein are released via exosomes (E. Emmanouilidou et al., Cell-produced alpha-synuclein is secreted in a calcium-dependent manner by exosomes and impacts neuronal survival. J Neurosci 30, 6838-6851 (2010); J. Ngolab et al., Brain-derived exosomes from dementia with Lewy bodies propagate alpha-synuclein pathology. Acta Neuropathol Commun 5, 46 (2017) as part of the prion-like cell-to-cell transmission. To determine whether irisin affects the cell-to-cell transmission of α-syn, we monitored the levels of α-syn PFF in the exosomal fraction and endolysomal fraction after treatment of cortical neurons with biotin-labeled α-syn PFF (α-syn-biotin) and irisin. α-Syn-biotin PFF was used to distinguish the biotin-labeled α-syn PFF from endogenous α-synuclein. Prior experiments have shown that α-syn-biotin behaves in manner similar to unlabeled α-synuclein PFF (X. Mao et al., Pathological α-synuclein transmission initiated by binding lymphocyte-activation gene 3. Science 353 (2016)). One hour pretreatment of cortical neurons with 50 ng/ml of irisin had no effect on the presence of α-syn-biotin PFF in the exosomal fraction (FIGS. 2A and 2B) of α-syn-biotin-PFF treated cortical neurons. On the other hand, one hour pretreatment of cortical neurons with 50 ng/ml of irisin significantly reduced the levels of α-syn-biotin PFF in the endolysomal fraction (FIGS. 2A and 2C). To determine whether internalized α-syn-biotin PFF is degraded via the lysosomal system or the ubiquitin proteasome system, the effect of the lysosomal inhibitor NH₄Cl and proteasome inhibitor MG132 was assessed. NH₄Cl prevented the degradation of α-syn-biotin PFF, while MG132 had no effect (FIG. 2D). One hour pretreatment of cortical neurons with 50 ng/ml of irisin significantly reduced the level of α-syn-biotin PFF in cortical neurons at 3 hours and 6 hours after treatment of cortical neurons with α-syn-biotin PFF (FIG. 2E). This reduction in the levels of α-synuclein-biotin PFF was reduced by NH₄Cl (FIG. 2E). One hour pretreatment of cortical neurons with 50 ng/ml of irisin had no effect on endogenous α-synuclein, while it reduced α-synuclein-biotin PFF levels (FIG. 2F).

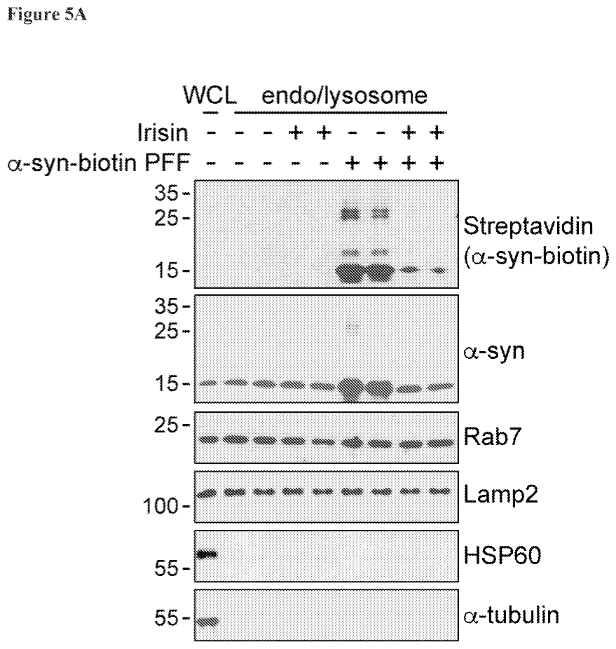

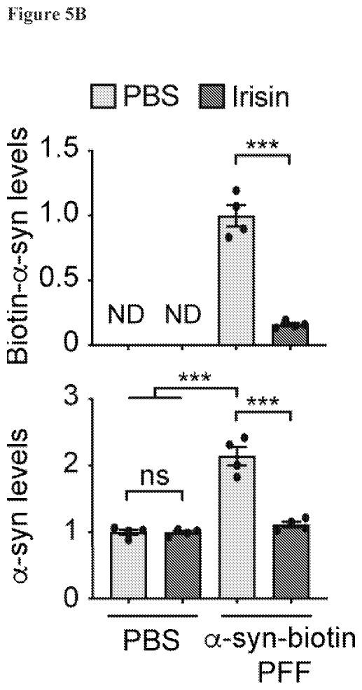

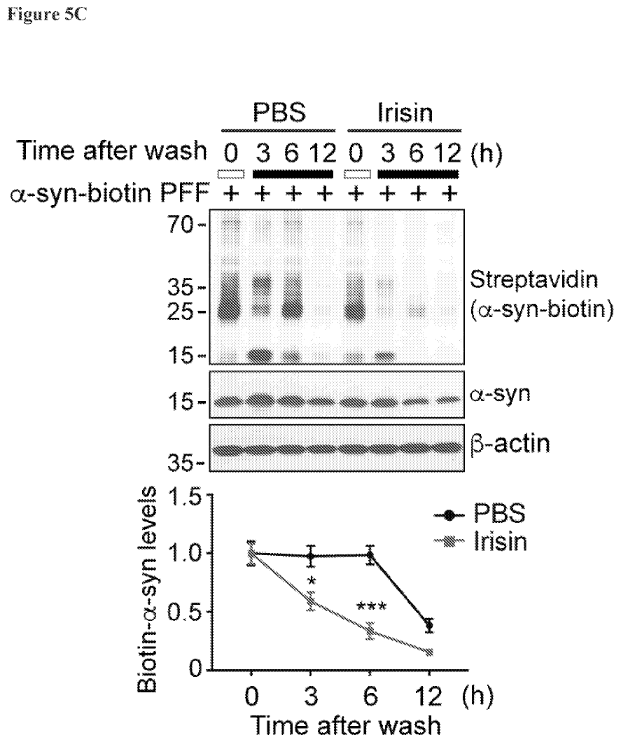

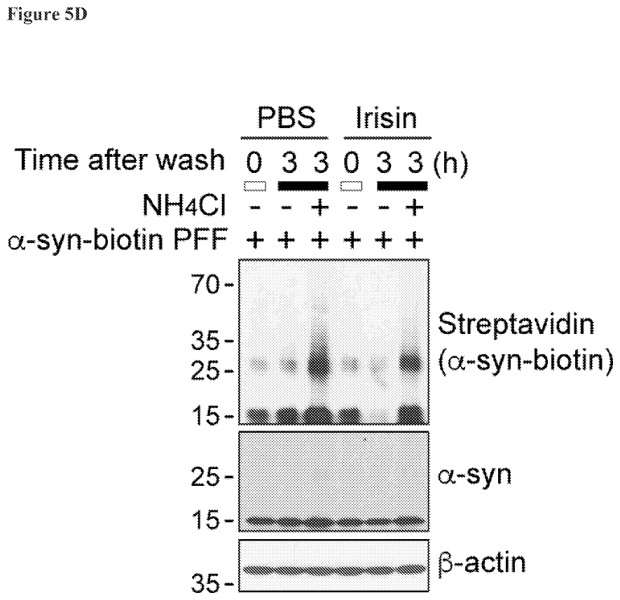

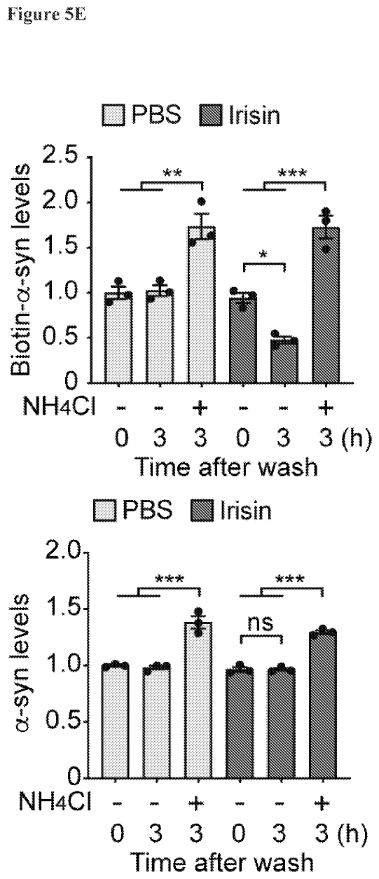

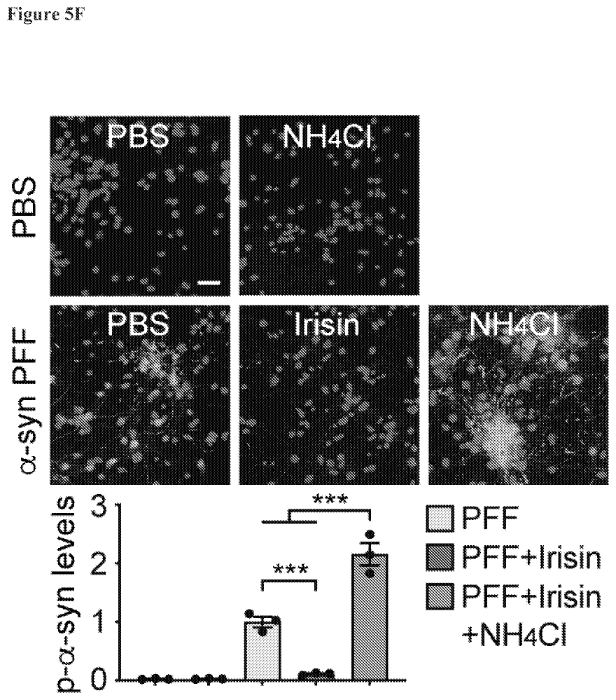

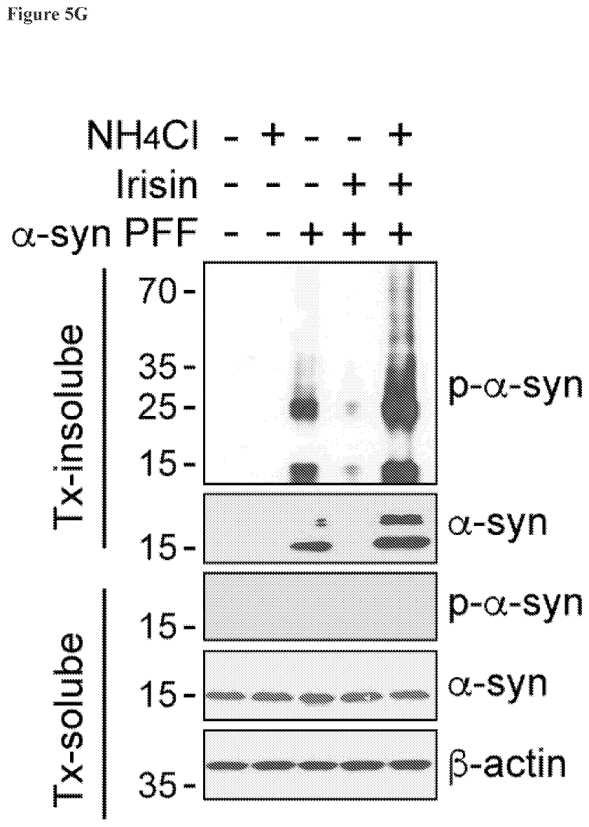

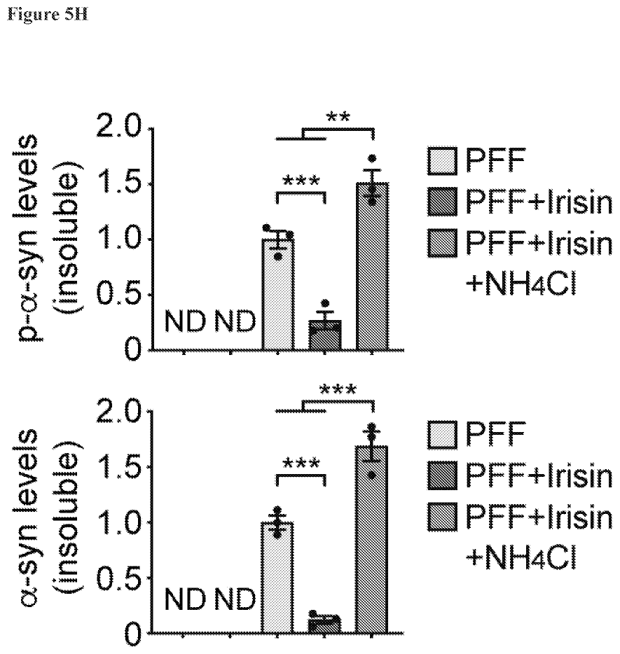

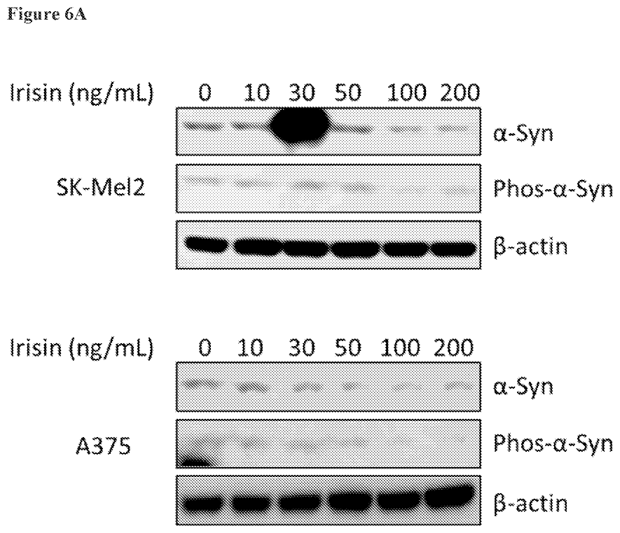

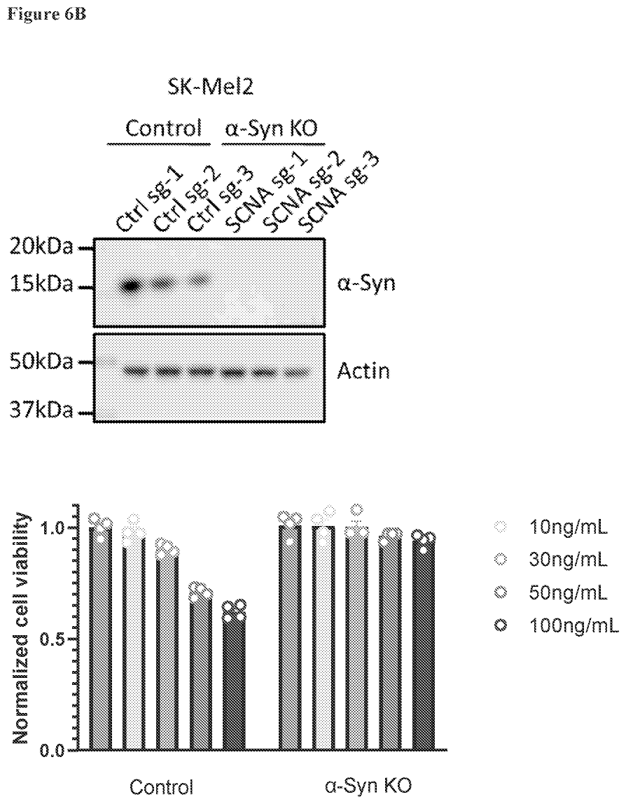

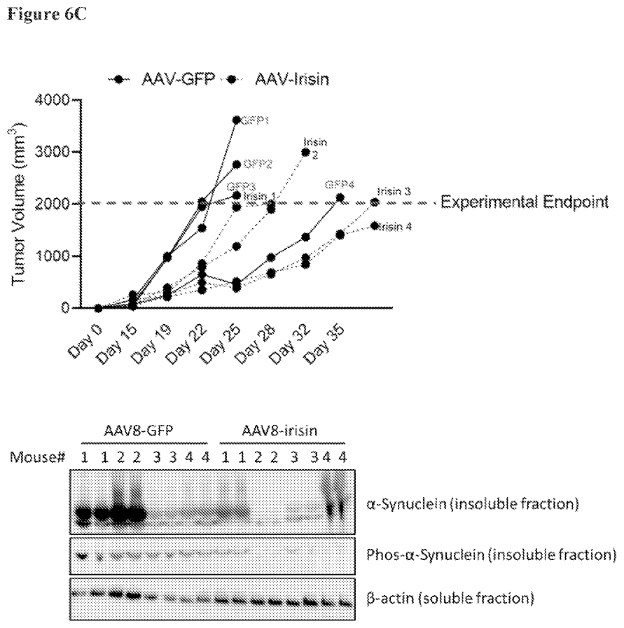

It was also contemplated whether irisin might inhibit the intracellular accumulation of α-syn by regulating endolysosomal degradation of α-syn. α-Syn PFF levels in the endolysosomes-containing fraction after treatment of primary cortical neurons with α-syn-biotin PFF and irisin were measured. One hour pre- and continuous treatment of cortical neurons with 50 ng/mL of irisin significantly reduced α-syn-biotin PFF levels in the endolysosomes-containing fraction (FIGS. 5A and 5B). To determine whether internalized α-syn-biotin PFF is degraded via the lysosomal system or through the ubiquitin proteasome system, the effect of the well-known lysosomal inhibitor NH₄Cl or the proteasome inhibitor MG132 affected the degradation of α-syn in the absence or presence of irisin was evaluated. In the absence of irisin, NH₄Cl prevented the degradation of α-syn-biotin PFF, while MG132 had no effect (FIG. 2D). To detect the endolysosomal degradation of internalized α-syn, primary cultured cortical neurons were pretreated with 50 ng/mL irisin for 1 hour and further incubated with 50 ng/mL irisin and α-syn-biotin PFF (1 μg/mL) for 12 hours. In this experiment, the medium was replaced with irisin and no α-syn-biotin PFF. This irisin treatment significantly reduced α-syn-biotin PFF levels in cortical neurons at 3 hours and 6 hours after the media replacement of cortical neurons (FIG. 5C). This reduction in the levels of α-syn-biotin PFF by irisin treatments was inhibited by NH₄Cl (FIGS. 5D and 5E) suggesting that irisin reduces pathologic α-syn by enhancing the pathway of lysosomal-mediated degradation. To confirm that irisin-induced lysosomal degradation of internalized α-syn PFF reduces pathologic α-syn, the effect of the irisin in the presence and absence of NH₄Cl on the formation of p-α-syn was monitored (FIG. 5F-5H). Primary cultured cortical neurons were treated with α-syn PFF for 2 days followed by treatment with 50 ng/mL of irisin in the presence and absence of a low dose of NH₄Cl, which had previously been shown to exhibit minimal neurotoxicity (A. Klejman et al., Mechanisms of ammonia-induced cell death in rat cortical neurons: Roles of NMDA receptors and glutathione. Neurochem. Int. 47, 51-57 (2005)). Posttreatment with irisin 2 days after α-syn PFF administration significantly reduced the levels of p-α-syn, while coadministration of NH₄Cl prevented the reduction of p-α-syn by irisin (FIG. 5F-5H).

These results taken together show that irisin prevented the pathologic transmission of α-synuclein by promoting the endolysosomal degradation of α-syn PFF.

### Irisin Prevents the Loss of DA Neurons in the Intrastriatal α-Synuclein PFF Model of PD.

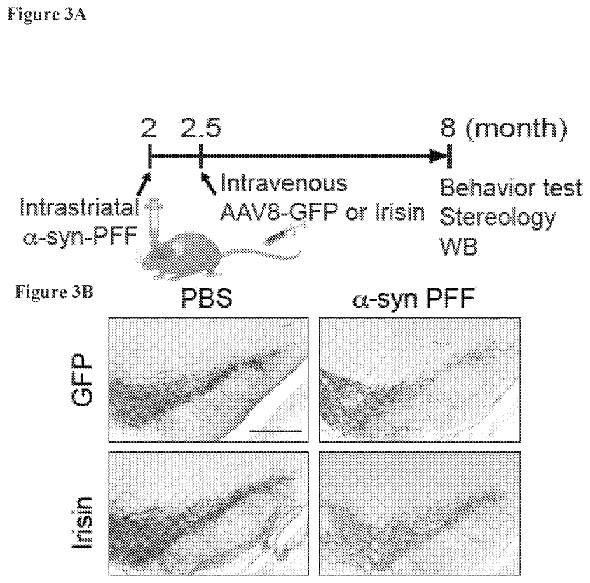

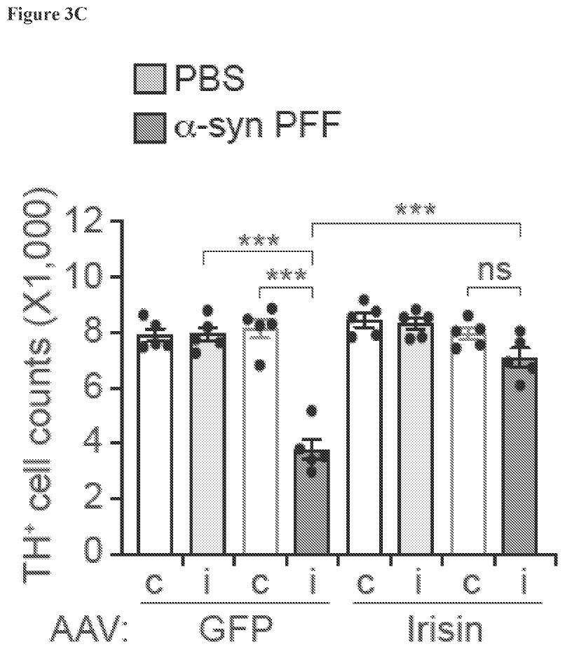

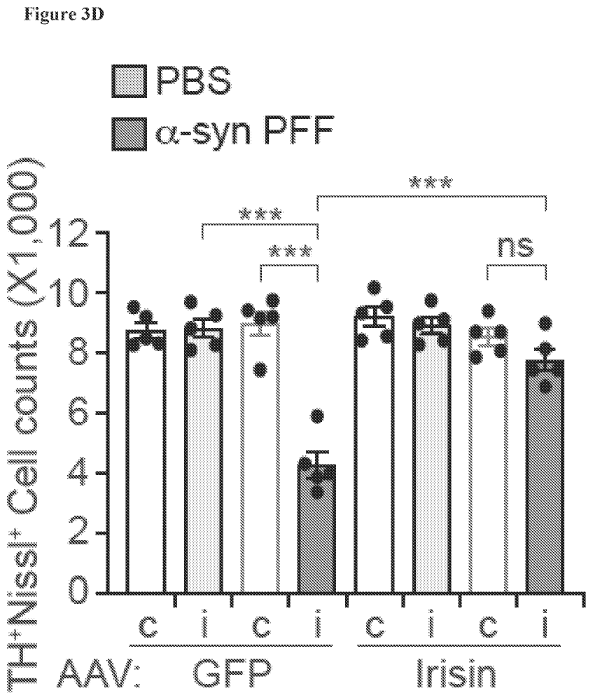

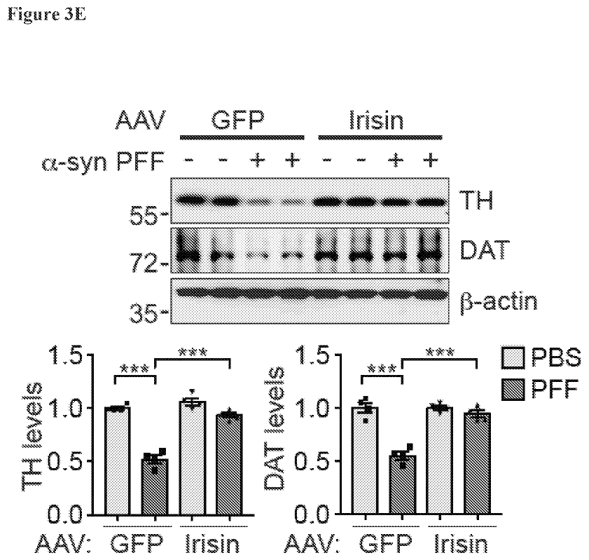

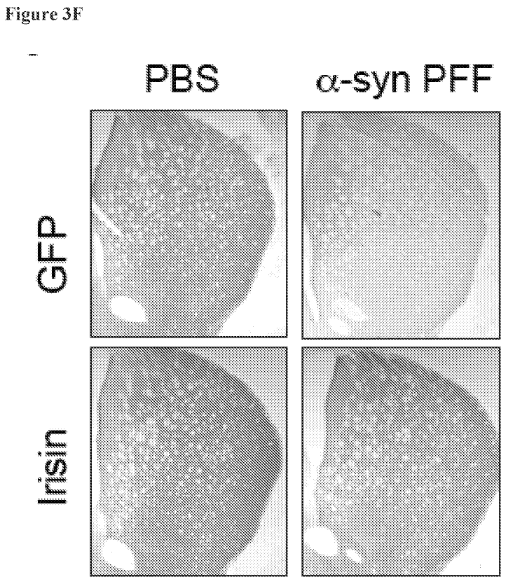

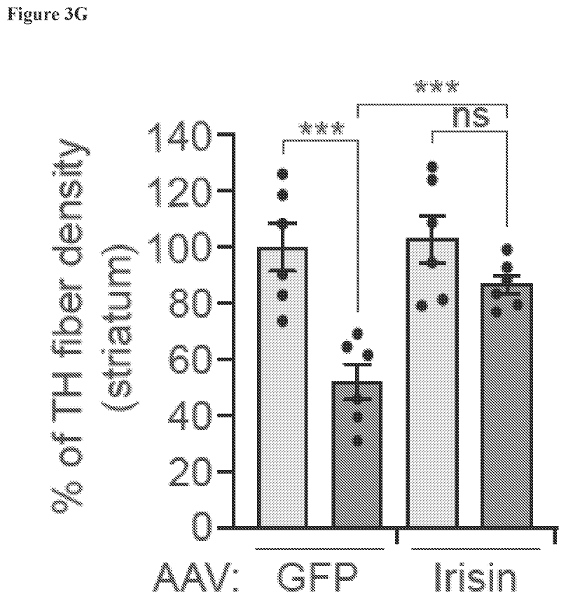

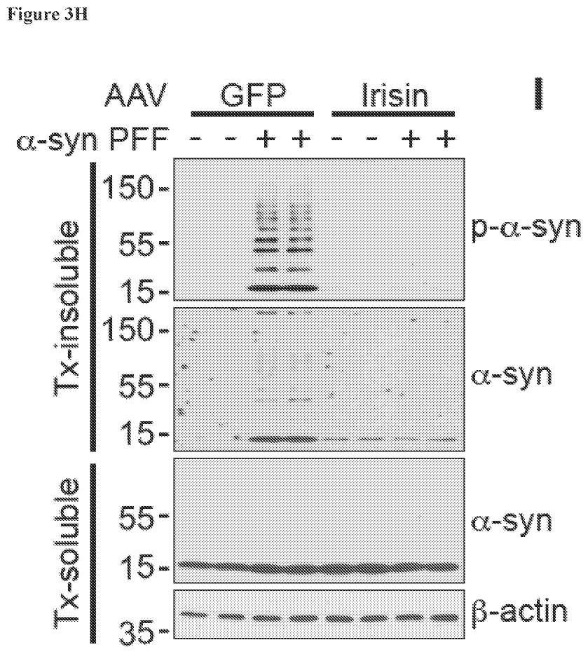

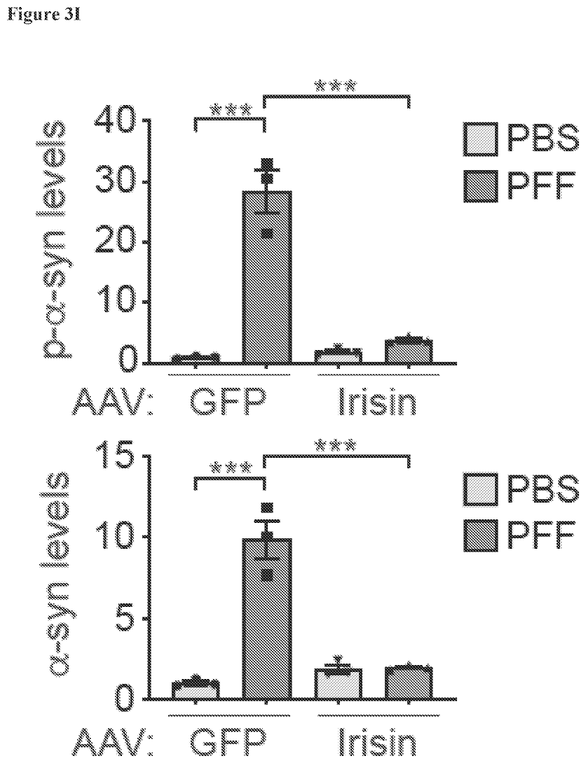

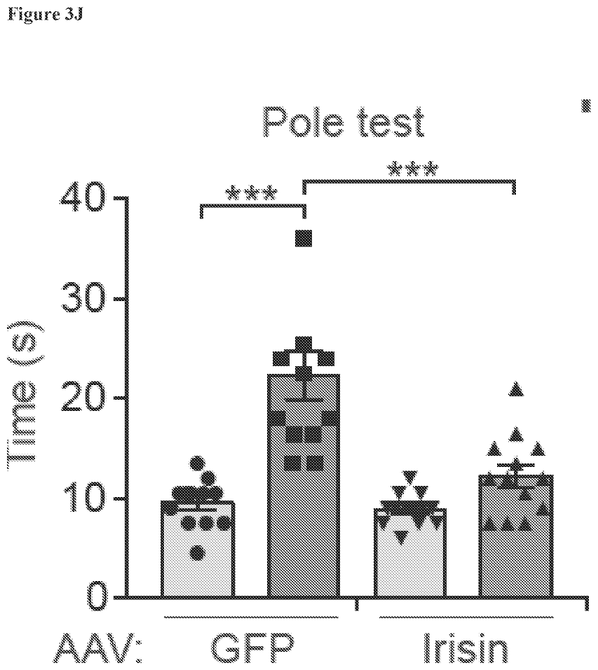

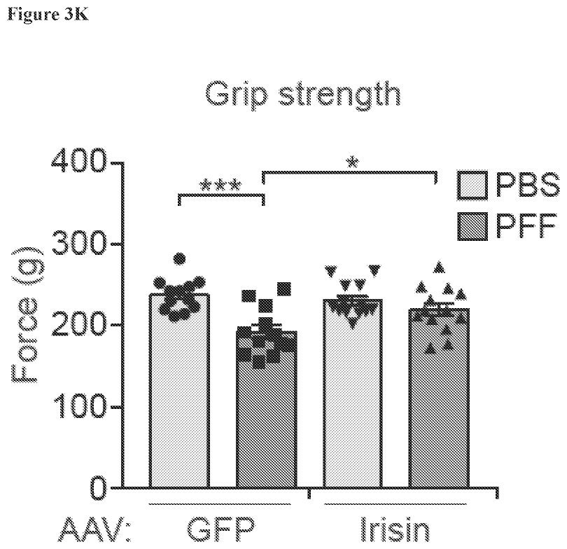

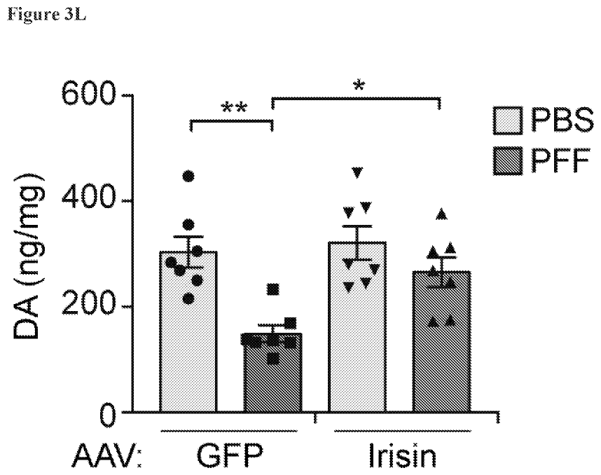

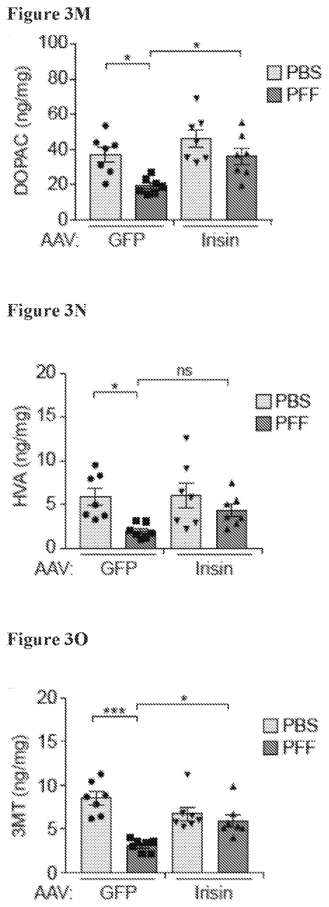

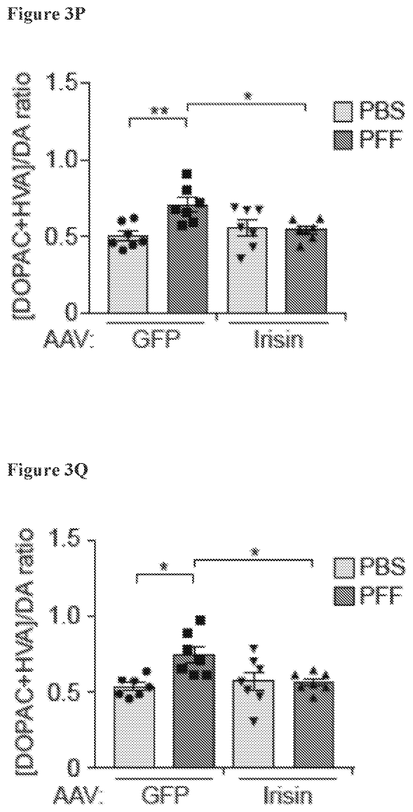

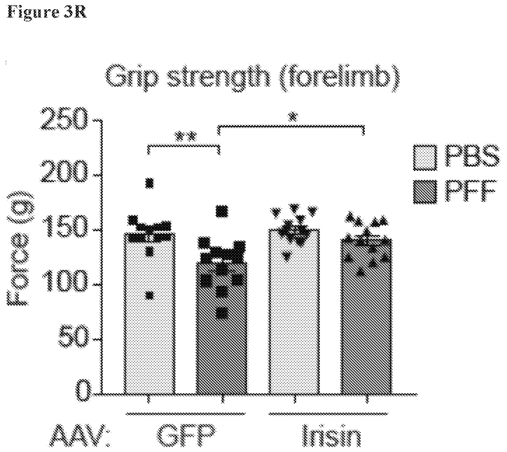

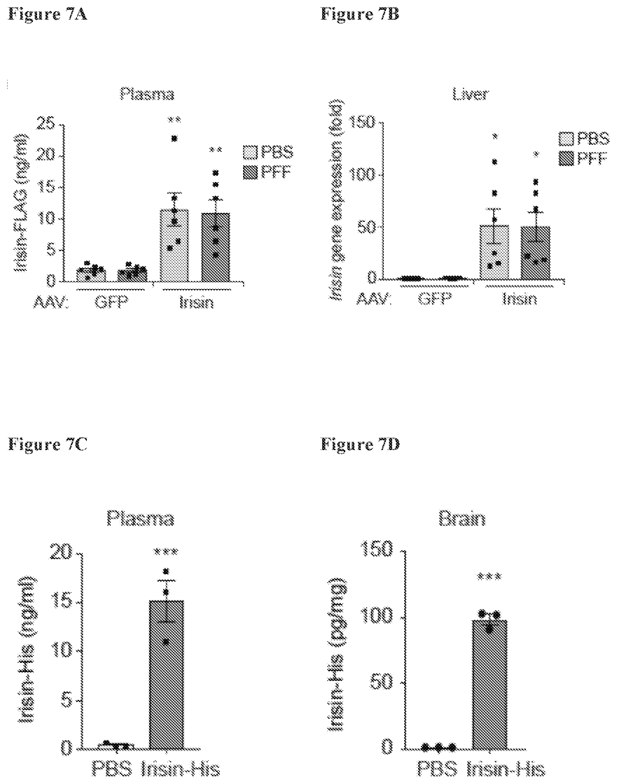

To determine whether irisin can prevent pathologic α-synuclein induced degeneration, α-synuclein PFF were stereotaxically injected into the striatum of mice (T. I. Kam et al., Poly(ADP-ribose) drives pathologic α-synuclein neurodegeneration in Parkinson's disease. Science 362 (2018); K. C. Luk et al., Pathological α-synuclein transmission initiates Parkinson-like neurodegeneration in nontransgenic mice. Science 338, 949-953 (2012). The protective role of irisin was evaluated by a AAV8-Irisin versus AAV8-GFP tail vein injection 2 weeks after the intrastriatal α-synuclein PFF injection (FIG. 3A). Prior studies indicate that this route of administration of irisin provides sufficient brain levels of irisin to reduce the pathology in 2 models of AD (M. R. Islam et al., Exercise hormone irisin is a critical regulator of cognitive function. Nat. Metab. 3, 1058-1070 (2021)). Importantly, this vector and does not infect and express within the brain (M. R. Islam et al., Exercise hormone irisin is a critical regulator of cognitive function. Nat. Metab. 3, 1058-1070 (2021)). 5.5 mo after the tail vein injection of AAV8-irisin-FLAG, we observed that irisin-FLAG was significantly elevated in the plasma and liver in both intrastriatal phosphate-buffered saline (PBS) and α-syn PFF injected mice (FIGS. 7A and 7B). Intravenous injection of irisin-His peptide (1 mg/kg) in mice, led to a significant elevation of irisin-His in plasma and brain, indicating that exogenous irisin is capable of increasing irisin levels in the brain by crossing the blood brain barrier (FIGS. 7C and 7D).

One month after the intrastriatal α-syn PFF injection, the pathogenic spread of α-synuclein to the substantia nigra has begun (T. I. Kam et al., Poly(ADP-ribose) drives pathologic α-synuclein neurodegeneration in Parkinson's disease. Science 362 (2018). As previously described (K. C. Luk et al., Pathological alpha-synuclein transmission initiates Parkinson-like neurodegeneration in nontransgenic mice. Science 338, 949-953 (2012); X. Mao et al., Pathological alpha-synuclein transmission initiated by binding lymphocyte-activation gene 3. Science 353 (2016)), WT mice showed an approximate 50% loss of DA neurons as assessed via non-biased stereologic counts of Tyrosine hydroxylase (TH) and Nissl stained neurons 6 months after a single intrastriatal injection of α-syn PFF (FIG. 3B-3D). AAV8-irisin injection prevented the loss of DA neurons when compared to AAV8-GFP injected mice (FIG. 3B-3D). Immunoblot analysis indicated that TH and dopamine transporter (DAT) levels were also reduced in response to α-synuclein PFF and this reduction was prevented by AAV8-irisin (FIG. 3E). Accompanying the loss of DA neurons, TH fiber density was reduced in striatum of α-syn PFF-injected mice with AAV8-GFP injection, but not with AAV8-irisin injection (FIG. 3F and FIG. 3G). High-performance liquid chromatography (HPLC) revealed that there was a reduction in striatal DA and its metabolites, 3,4-dihydroxyphenylacetic acid (DOPAC), homovanillic acid (HVA), and 3-methoxytyramine (3-MT) in α-syn PFF-injected mice. This loss of byproducts of dopamine and its metabolites were blocked by 87%, 95%, 72% and 70%, respectively in AAV8-Irisin injected mice (FIG. 3L and FIG. 3M-3O). DA turnover was increased in striatal α-syn PFF injected mice with AAV8-GFP injection, while these effects were suppressed in AAV8-irisin injected mice (FIG. 3P and FIG. 3Q). AAV8-irisin prevented the accumulation of insoluble pathologic p-α-synuclein and α-synuclein compared AAV8-GFP treated mice, while having no effects on soluble α-synuclein monomer levels (FIGS. 3H and 3I). In addition, AAV8-irisin prevented the α-synuclein PFF-induced behavioral deficits on the pole test (FIG. 3J) and grip strength test (FIG. 3K and FIG. 3R). Taken together, these results indicate that irisin prevents the loss of DA neurons and the neurobehavioral deficits induced striatal α-synuclein PFFs.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments encompassed by the present invention described herein. Such equivalents are intended to be encompassed by the following claims.

1-31. (canceled)

32. A method of preventing or treating α-synucleinopathy in a human subject, the method comprising administering to the human subject an effective amount of an agent selected from the group consisting of i) an Fndc5 polypeptide or a biologically active fragment thereof, and ii) a nucleic acid that encodes an Fndc5 polypeptide or a biologically active fragment thereof, wherein the α-synucleinopathy is pathologic α-synuclein-induced Parkinson's disease.

33. The method of claim 32, wherein the agent is selected from the group consisting of:
a) a polypeptide comprising an amino acid sequence of SEQ ID NO: 2, wherein said fragment lacks the C-terminal domain sequence of said FNDC5 polypeptide, and wherein said polypeptide has one or more of the biological activities of said FNDC5 polypeptide;
b) a polypeptide comprising an amino acid sequence of residues 73-140 of the FNDC5 polypeptide of SEQ ID NO:2, wherein said polypeptide does not encode the C-terminal domain sequence of said FNDC5 polypeptide, and wherein said polypeptide has one or more of the biological activities of said FNDC5 polypeptide;
c) a polypeptide fragment of the FNDC5 polypeptide of SEQ ID NO: 2, wherein the fragment consists of a sequence of amino acids in between residues 1 and 150 of SEQ ID NO: 2, and wherein the fragment has one or more of the biological activities of said FNDC5 polypeptide; and
d) a polypeptide fragment of FNDC5 comprising an amino acid sequence of a fragment of a FNDC5 polypeptide of SEQ ID NO: 4, 6 or 8, and wherein said fragment has one or more of the biological activities of said FNDC5 polypeptide.

34. The method of claim 32, wherein
(a) the polypeptide is fused to one or more heterologous polypeptides at its N-terminus and/or C-terminus; and/or
(b) the polypeptide comprises an amino acid modification, post-translational modification, and/or a heterologous an amino acid sequence, that stabilizes the polypeptide and/or increases its half-life, optionally wherein the one or more heterologous polypeptides is an Fc domain or fragment thereof.

35. The method of claim 32, wherein the agent is a nucleic acid that encodes
a) a polypeptide comprising an amino acid sequence of SEQ ID NO: 2, wherein said fragment lacks the C-terminal domain sequence of said FNDC5 polypeptide, and wherein said polypeptide has one or more of the biological activities of said FNDC5 polypeptide;
b) a polypeptide comprising an amino acid sequence of residues 73-140 of the FNDC5 polypeptide of SEQ ID NO:2, wherein said polypeptide does not encode the C-terminal domain sequence of said FNDC5 polypeptide, and wherein said polypeptide has one or more of the biological activities of said FNDC5 polypeptide;
c) a polypeptide which is a fragment of the FNDC5 polypeptide of SEQ ID NO: 2, wherein the fragment consists of a sequence of amino acids in between residues 1 and 150 of SEQ ID NO: 2, and wherein the fragment has one or more of the biological activities of said FNDC5 polypeptide; or
d) a polypeptide which is a fragment of FNDC5 comprising an amino acid sequence of a fragment of a FNDC5 polypeptide of SEQ ID NO: 4, 6 or 8, and wherein said fragment has one or more of the biological activities of said FNDC5 polypeptide.

36. The method of claim 32, wherein the agent is a nucleic acid that encodes
(a) the polypeptide which is fused to one or more heterologous polypeptides at its N-terminus and/or C-terminus; and/or
(b) the polypeptide comprising an amino acid modification, post-translational modification, and/or a heterologous an amino acid sequence, that stabilizes the polypeptide and/or increases its half-life, optionally wherein the one or more heterologous polypeptides is an Fc domain or fragment thereof.

37. The method of claim 32, wherein the nucleic acid is comprised within an expression vector.

38. The method of claim 37, wherein the expression vector is a viral expression vector.

39. The method of claim 38, wherein
(a) the viral expression vector is an adeno-associated viral (AAV) vector; and/or
(b) each dose of the viral expression vector comprises at least 1.0×10¹¹ GC/kg particles.

40. The method of claim 32, wherein
(a) the agent is administered systemically, optionally wherein systemic administration is intravenous or subcutaneous;
(b) the agent is administered in a pharmaceutically acceptable formulation;
(c) the agent is administered at least once a day, at least one a week, or at least once a month;
(d) the agent is administered to the subject for greater than a number of months equal to 3, 4, 5, 6, 7, 8, 9, 10, 11, 12, 13, 14, 15, 16, 17, 18, 19, 20, 21, 22, 23, or 24, optionally the agent is administered to the subject for the duration of the subject's life;
(e) the agent crosses the blood brain barrier;
(f) the agent does not increase the levels of brain-derived neurotrophic factor (BDNF) in neurons in the subject over the course of treatment;
(g) prevents or reduces degeneration of dopaminergic (DA) neurons in the subject;
(h) blocks the accumulation of or decreases the amount of α-synuclein in neurons or glia in the subject; and/or
(i) the agent decreases at least one motor deficit, optionally wherein the at least one motor deficit is selected from the group consisting of i) tremor at rest, such as a slight tremor in the hands or feet, ii) rigidity (stiffness) of limbs, neck, or shoulders, iii) difficulty balancing (postural instability), iv) slowness of movement or gradual loss of spontaneous movement (bradykinesia), v) trouble standing after sitting, vi) stiffness in the limbs, and vii) moving more slowly.

41. A method of preventing or treating α-synucleinopathy in a human subject, the method comprising administering to the human subject an effective amount of an agent selected from the group consisting of i) an Fndc5 polypeptide comprising the amino acid sequence of residues 73-140 SEQ ID NO:2, and ii) a nucleic acid that encodes an Fndc5 polypeptide comprising the amino acid sequence of residues 73-140 SEQ ID NO:2,
wherein the α-synucleinopathy is pathologic α-synuclein-induced Parkinson's disease.

42. The method of claim 41, wherein the agent is a polypeptide and polypeptide
(a) lacks the C-terminal domain sequence of said FNDC5 polypeptide.
(b) is fused to one or more heterologous polypeptides at its N-terminus and/or C-terminus; and/or
(c) comprises an amino acid modification, post-translational modification, and/or a heterologous an amino acid sequence, that stabilizes the polypeptide and/or increases its half-life, optionally wherein the one or more heterologous polypeptides is an Fc domain or fragment thereof.

43. The method of claim 41, wherein the agent is a nucleic acid that encodes an Fndc5 polypeptide comprising the amino acid sequence of residues 73-140 SEQ ID NO:2.

44. The method of claim 43, wherein the said FNDC5 polypeptide.
(b) is fused to one or more heterologous polypeptides at its N-terminus and/or C-terminus; and/or
(c) comprises an amino acid modification, post-translational modification, and/or a heterologous an amino acid sequence, that stabilizes the polypeptide and/or increases its half-life, optionally wherein the one or more heterologous polypeptides is an Fc domain or fragment thereof.

45. The method of claim 41, wherein the nucleic acid is comprised within an expression vector.

46. The method of claim 45, wherein the expression vector is a viral expression vector.

47. The method of claim 46, wherein
(a) the viral expression vector is an adeno-associated viral (AAV) vector; and/or
(b) each dose of the viral expression vector comprises at least 1.0×10¹¹ GC/kg particles.

48. The method of claim 41, wherein
(a) the agent is administered systemically, optionally wherein systemic administration is intravenous or subcutaneous;
(b) the agent is administered in a pharmaceutically acceptable formulation;
(c) the agent is administered at least once a day, at least one a week, or at least once a month;
(d) the agent is administered to the subject for greater than a number of months equal to 3, 4, 5, 6, 7, 8, 9, 10, 11, 12, 13, 14, 15, 16, 17, 18, 19, 20, 21, 22, 23, or 24, optionally the agent is administered to the subject for the duration of the subject's life;
(e) the agent crosses the blood brain barrier;
(f) the agent does not increase the levels of brain-derived neurotrophic factor (BDNF) in neurons in the subject over the course of treatment;
(g) prevents or reduces degeneration of dopaminergic (DA) neurons in the subject;
(h) blocks the accumulation of or decreases the amount of α-synuclein in neurons or glia in the subject; and/or
(i) the agent decreases at least one motor deficit, optionally wherein the at least one motor deficit is selected from the group consisting of i) tremor at rest, such as a slight tremor in the hands or feet, ii) rigidity (stiffness) of limbs, neck, or shoulders, iii) difficulty balancing (postural instability), iv) slowness of movement or gradual loss of spontaneous movement (bradykinesia), v) trouble standing after sitting, vi) stiffness in the limbs, and vii) moving more slowly.

49. A method of preventing or treating α-synucleinopathy in a human subject, the method comprising administering to the human subject an effective amount of an agent selected from the group consisting of i) an Fndc5 polypeptide or a biologically active fragment thereof, and ii) a nucleic acid that encodes an Fndc5 polypeptide or a biologically active fragment thereof, wherein the α-synucleinopathy is α-synuclein-induced Alzheimer's disease or α-synuclein-induced symptoms thereof.

50. A method of preventing or treating α-synucleinopathy in a human subject, the method comprising administering to the human subject an effective amount of an agent selected from the group consisting of i) an Fndc5 polypeptide or a biologically active fragment thereof, and ii) a nucleic acid that encodes an Fndc5 polypeptide or a biologically active fragment thereof, wherein the α-synucleinopathy is multiple system atrophy (MSA).

51. A method of preventing or treating α-synucleinopathy in a human subject, the method comprising administering to the human subject an effective amount of an agent selected from the group consisting of i) an Fndc5 polypeptide or a biologically active fragment thereof, and ii) a nucleic acid that encodes an Fndc5 polypeptide or a biologically active fragment thereof, wherein the α-synucleinopathy is neuroaxonal dystrophy.


SEQUENCE LISTING (16 sequences)

SEQ ID NO: 1 - DNA, 630 bp, Mus musculus
  source 1..630: mol_type = genomic DNA
atgcccccagggccgtgcgcctggccgccccgcgccgcgctccgcctgtggctaggctgcgtctgcttcgcgctggtgcaggcggacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggagatggggaggaaccagcagctgcgaacgggggaggtgctgatcattgttgtggtcctcttcatgtgggcaggtgttatagctctcttctgccgccagtatgatatcatcaaggacaacgagcccaataacaacaaggagaaaaccaagagcgcatcagaaaccagcacaccggagcatcagggtgggggtctcctccgcagcaagatatga

SEQ ID NO: 2 - protein, 209 aa, Mus musculus
MPPGPCAWPPRAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 3 - DNA, 462 bp, Homo sapiens
  source 1..462: mol_type = genomic DNA
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcatcaaggacaatgaacccaataacaacaaggaaaaaaccaagagtgcatcagaaaccagcacaccagagcaccagggcggggggcttctccgcagcaaggtgagggcaagacctgggcctgggtgggccaccctgtgcctcatgctctggtaa

SEQ ID NO: 4 - protein, 153 aa, Homo sapiens
MLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKVRARPGPGWATLCLMLW

SEQ ID NO: 5 - DNA, 414 bp, Homo sapiens
  source 1..414: mol_type = genomic DNA
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcatcaaggacaatgaacccaataacaacaaggaaaaaaccaagagtgcatcagaaaccagcacaccagagcaccagggcggggggcttctccgcagcaagatatga

SEQ ID NO: 6 - protein, 137 aa, Homo sapiens
MLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 7 - DNA, 321 bp, Homo sapiens
  source 1..321: mol_type = genomic DNA
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcattgaagcgtga

SEQ ID NO: 8 - protein, 106 aa, Homo sapiens
MLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIEA

SEQ ID NO: 9 - DNA, 1203 bp, Gallus gallus
  source 1..1203: mol_type = genomic DNA
atggagaagaacagggacggccgcggcccccctggtgtccatctggggatggagaaggaagatgatttagagcccggtgacacgccggggctgcgcgaagccctggtggcgagatgtcaccgctgccgcgcacccgccgggggtctcaccgggacgggccccgtttgctccttccggcgatggggagcggtccgggccgagggctcccggtcccgcctgggggaaactgaggcagacggcggggccgggcggggcgggggccgagccgcccccgggccgggggagggaccggagcggggctgcccagcgctgcagcgggcggagccggggctcggcggggccgcctcccggccgagccgagccgaaccgagccgcgctgccgagggccgccgagcccgcagccgcccccggccgaaccgggcggccccgccggttccgggccccggagctctccgcggtgctgaacggcgccgccgcgcccgcgggacgccggccccggagcggctcggccccggcgcggcgcggcgggccgcggggggatggagcccttcctgggctgcaccggcgccgcgctcctgctctgctttcagctacgccggtctgcggccggtggaggcagacagcccttcggctccggtcaatgtcacagtcaaacacctgaaggccaactcagctgtagtgacttgggacgttctggaggatgaagttgtcattggatttgccatttcccagcagaagaaggacgtgcggatgctgcgcttcatccaggaggtgaacaccaccacccgctcctgtgccctctgggacctagaggaggacactgagtacattgtgcatgtccaggccatcagcatccaaggccagagccctgccagtgagccagtcctcttcaagacccccagggaagctgagaaactggcttctaaaaataaagatgaggtgacaatgaaggagatggcgaagaaaaaccaacagctgcgcgcaggggaaatactcatcattgtggtggtgttgtttatgtgggcaggggtgatcgccctgttctgcaggcagtacgacatcatcaaagacaacgagccgaacaacagcaaggagaaagccaagagcgcctcagagaacagcacccccgagcaccagggtggggggctgctccgcagcaagttcccaaaaaacaaaccctcagtgaacatcattgaggcataa

SEQ ID NO: 10 - protein, 400 aa, Gallus gallus
MEKNRDGRGPPGVHLGMEKEDDLEPGDTPGLREALVARCHRCRAPAGGLTGTGPVCSFRRWGAVRAEGSRSRLGETEADGGAGRGGGRAAPGPGEGPERGCPALQRAEPGLGGAASRPSRAEPSRAAEGRRARSRPRPNRAAPPVPGPGALRGAERRRRARGTPAPERLGPGAARRAAGGWSPSWAAPAPRSCSAFSYAGLRPVEADSPSAPVNVTVKHLKANSAVVTWDVLEDEVVIGFAISQQKKDVRMLRFIQEVNTTTRSCALWDLEEDTEYIVHVQAISIQGQSPASEPVLFKTPREAEKLASKNKDEVTMKEMAKKNQQLRAGEILIIVVVLFMWAGVIALFCRQYDIIKDNEPNNSKEKAKSASENSTPEHQGGGLLRSKFPKNKPSVNIIEA

SEQ ID NO: 11 - DNA, 555 bp, Danio rerio
  source 1..555: mol_type = genomic DNA
atgagttcttacagtttggcagctccagtgaatgtgtccatcagggatctgaagagcagctcagccgtggtgacatgggacacgccagacggagagccagtcatcggcttcgccatcacacaacagaagaaagatgtccgcatgctgcgctttattcaagaagtgaacaccaccacgcggagctgtgcattgtgggatctggaagctgatacggattacattgtgcacgttcagtctatcagcatcagcggggcgagtcctgttagtgaagctgtgcacttcaagaccccgacagaagttgaaacacaggcctccaagaacaaagacgaggtgacgatggaggaggtcgggccgaacgctcagctcagggccggagagttcatcattattgtggtggtcctcatcatgtgggcaggtgtgatcgcactattctgccgtcagtatgacatcattaaagacaacgaaccaaacaataacaaggataaagccaagaactcgtctgaatgcagcactccagagcacacgtcaggtggcctgctgcgcagtaaggtataa

SEQ ID NO: 12 - protein, 184 aa, Danio rerio
MSSYSLAAPVNVSIRDLKSSSAVVTWDTPDGEPVIGFAITQQKKDVRMLRFIQEVNTTTRSCALWDLEADTDYIVHVQSISISGASPVSEAVHFKTPTEVETQASKNKDEVTMEEVGPNAQLRAGEFIIIVVVLIMWAGVIALFCRQYDIIKDNEPNNNKDKAKNSSECSTPEHTSGGLLRSKV

SEQ ID NO: 13 - DNA, 336 bp, Mus musculus
  source 1..336: mol_type = genomic DNA
gacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggag

SEQ ID NO: 14 - protein, 112 aa, Mus musculus
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 15 - DNA, 336 bp, Homo sapiens
  source 1..336: mol_type = genomic DNA
gacagtccctcagccccagtgaacgtcaccgtcaggcacctcaaggccaactctgcagtggtgagctgggatgttctggaggatgaggttgtcatcggatttgccatctcccagcagaagaaggatgtgcggatgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagag

SEQ ID NO: 16 - protein, 6 aa, synthetic construct
HHHHHH